(19) United States
(12) Patent Application Publication
(10) Pub. No.: US 2026/0337693 A1
(43) Pub. Date: Oct. 29, 2026

(54) ENGINEERED T CELL ENGAGER RECEPTORS RECOGNIZING TP53 R273H PEPTIDE-HLA-C*01:02 COMPLEXES AND METHODS OF IDENTIFICATION

(71) Applicant: NHAN THANH HUYNH, Lam Dong (VN)

(72) Inventors: NHAN THANH HUYNH, Lam Dong (VN); MY THI THU NGUYEN, Tay Ninh (VN); NGOC THI BUI, Dong Nai (VN); HOAI NGHIA NGUYEN, Ho Chi Minh (VN); CUONG THACH NGUYEN, Ho Chi Minh (VN)

(73) Assignee: NEXCALIBUR THERAPEUTICS, CORP., LEWES, DE (US)

(21) Appl. No.: 19/533,283

(22) Filed: Feb. 8, 2026

Related U.S. Application Data
(63) Continuation of application No. 19/059,303, filed on Feb. 21, 2025, now Pat. No. 12,442,046.

(51) Int. Cl. C12Q 1/6886; C07K 14/705; G01N 33/50

(52) U.S. Cl. CPC C12Q 1/6886; C07K 14/7051; G01N 33/505; C12Q 2600/156; C12Q 2600/158; G01N 2333/7051

(57) ABSTRACT

A method for identifying and optimizing an engineered T cell engager receptor (TCE) specifically recognizing a peptide-major histocompatibility complex (pMHC) comprising HLA-C*01:02 presenting a TP53 R273H-derived 9-mer peptide, comprising: (a) generating a plurality of candidate optimized T cell receptor (TCR) sequences by modifying a TCR sequence capable of recognizing the pMHC comprising HLA-C+01:02 presenting the TP53 R273H-derived 9-mer peptide; (b) generating a plurality of candidate TCE constructs by incorporating the candidate optimized TCR sequences into TCE constructs; and (c) evaluating each candidate TCE construct based on three criteria to identify a TCE specifically recognizing the pMHC, wherein the TCE specifically recognizing the pMHC comprising HLA-C*01:02 presenting the TP53 R273H-derived 9-mer peptide is encoded by a nucleic acid or is a polypeptide.

### INCORPORATION BY REFERENCE OF MATERIAL SUBMITTED ELECTRONICALLY

The Sequence Listing, which is a part of the present disclosure, is submitted concurrently with the specification as an XML file in compliance with 37 CFR 1.831-1.835. The contents of the electronic sequence listing (TCE_001_Seqlisting.xml; size: 182,781 bytes; and date of creation: Feb. 5, 2026) are herein incorporated by reference in its entirety.

### CLAIM OF PRIORITY

This application is a continuation application of application Ser. No. 19/059,303, entitled “Method for identifying a shared neoantigen-reactive T cell receptor”, filed on Feb. 21, 2025. The patent application identified above is incorporated here by reference in its entirety to provide continuity of disclosure.

### FIELD OF THE INVENTION

The present invention relates to the field of immunology and protein engineering. More specifically, the invention relates to engineered T cell engager receptors (TCEs) that recognize peptide-major histocompatibility complexes (pMHCs), and to methods for identifying and optimizing such engineered receptors. In particular, the invention relates to engineered TCEs that specifically recognize a pMHC comprising HLA-C*01:02 presenting a TP53 R273H-derived peptide, as well as nucleic acids, polypeptides, and expression constructs encoding or comprising such engineered TCEs.

### BACKGROUND ART

Recent advances in immunology and cancer research have significantly accelerated the development of new cancer immunotherapies at a much higher pace and efficiency. T cell-based cancer immunotherapies can be mainly categorized into two major classes: therapies targeting immunosuppressive factors such as immune checkpoint inhibitors, and therapies promoting immunostimulatory pathways, including Chimeric Antigen Receptor (CAR) T cells and BiSpecific T cell engaging AntiBodies (bsAbs).

Antibodies have long been developed as one of the powerful tools for fighting cancer, especially bsAbs with the ability to redirect T cells against target cells through the unique recognition of tumor-specific antigens (TSAs) or TAAs on cancer cells and cell surface molecules on T cells. Importantly, TSAs can be presented as peptides on the tumor cell surface in major histocompatibility complex (MHC) proteins, which interact with the T-cell receptor (TCR) to stimulate an anti-tumor response. The TCR exhibits an antibody-like structure and the engineering of TCR-mimicking antibodies have provided greater potential for the development of T cell-redirected bsAbs. This TCE class set its position as intermediate between engineered CAR-T cells and bsAbs, and emerged as a promising anti-cancer tool. With their unique feature of specific recognition of TSAs, TCEs bridge T cells to cancer cells, thereby enhancing T cell-mediated killing efficiency.

Somatic mutations have been identified as common drivers of oncogenesis. Among these, mutations in genes encode for tumor suppressor proteins as those modifications can result in inactive or hyperactive versions of such enzymes. TP53 is one of the most frequently mutated gene in human cancer with R273H as one of the hotspots locates at the DNA-binding domain of the p53 protein. Even though being one of the most well-studied tumor suppressor proteins, only a limited number of drugs targeting mutant p53 have been approved for treatment as traditional drugs or antibodies cannot reach these intracellular targets. On the other hand, this mutation results in the presentation of neo-antigenic peptides on the surface of tumor cells via Major Histocompatibility Complex (MHC) molecule, such as HLA-C*01:02. This makes bispecific TCE become a promising approach to induce tumor cell lysis.

As per Patent No. WO2024197072A2, the patent provides methods for identifying novel neoantigen-reactive T cell receptors (TCRs) by co-culturing a reporter T cell comprising a TCR expression cassette and an antigen presenting cell expressing a target neoantigen sequence and a matched human lymphocyte antigen (HLA) sequence comprising: (i) obtaining single-cell gene expression profiles from a population of tumor infiltrating lymphocytes (TIL) isolated from a patient sample; (ii) performing bioinformatics analyses on the single cell gene expression data to identify TCR clonotypes of interests; (iii) creating recombinant TCR sequences; (iv) preparing a reporter T cell comprising a TCR expression cassette encoding a TCR sequence reconstructed from paired TCR α and β chain sequences identified from the clonotype of interest in step ii; (v) preparing a tandem minigene (TMG) expression vector; (vi) analyzing the patient sequencing data to identify class I and class IIHLA alleles and preparing HLA expression vectors comprising the class IHLA and class IIHLA allele sequences; (vii) preparing an antigen presenting cell (APC) comprising transecting the TMG expression vector and one or more HLA expression vectors into a cell wherein each transection condition comprises a TMG and one or two HLA types; (viii) co-culturing the reporter T cell in step iii with the APC of step vii; and (ix) identifying a positive reporter activity in the reporter T cell to identify a neoantigen-reactive TCR.

As per Patent No. US20240052010A1 refers to a method of obtaining a plurality of T cell receptors (TCRs) specifically recognizing a target tumor antigen peptide, comprising: (a) a first co-culturing step comprising co-culturing a first population of dendritic cells (DCs) loaded with the target tumor antigen peptide with a population of T cells from an individual to obtain a first co-culture; (b) an enrichment step comprising subjecting the first co-culture to an enrichment process to obtain enriched activated T cells; (c) a second co-culturing step comprising co-culturing the enriched activated T cells with a second population of DCs loaded with the target tumor antigen peptide to obtain a population of tumor antigen-specific T cells, wherein at least about 10% of the tumor antigen-specific T cells specifically responds to the target tumor antigen peptide; and (d) a sequencing step, comprising subjecting the tumor antigen-specific T cells to next-generation sequencing to identify a plurality of pairs of genes encoding TCRα and TCRβ, thereby providing the plurality of T cell receptors based on paired genes encoding TCRα and TCRβ; wherein the individual has clinically benefitted from a Multiple Antigen Specific Cell Therapy (MASCT) comprising administering to the individual an effective amount of activated T cells prepared by co-culturing a population of T cells with a population of dendritic cells loaded with a plurality of tumor antigen peptides comprising the target tumor antigen peptide.

The aforementioned inventions fulfill specific purposes and meet the requirements of a technical solution. However, existing approaches often fail to provide integrated workflows seamlessly combining neoantigen discovery, TCR prioritization, and functional validation within a unified process. Furthermore, many current methods prioritize sequence identification while placing insufficient emphasis on functional testing. In particular, functional parameters such as binding characteristics to defined pMHC complexes, induction of T cell activation, T cell-mediated cytotoxicity, and assessment of off-target reactivity are frequently addressed in a fragmented or non-integrated manner. Furthermore, the impact of receptor architecture, domain arrangement, and molecular configuration on these functional outcomes is not consistently evaluated within a unified framework.

Accordingly, there remains a need for improved and integrated strategies that enable the identification, optimization, and functional evaluation of engineered T cell engager receptors directed against defined pMHC targets, including mutant TP53-derived peptide-HLA complexes, while enabling assessment of specificity, functional activity, and reduced off-target effects.

The invention provides solutions to achieve the above objectives.

### SUMMARY OF THE INVENTION

The patent or application file contains at least one drawing executed in color. Copies of this patent or patent application publication with color drawing(s) will be provided by the Office upon request and payment of the necessary fee.

The present invention relates to engineered T cell engager receptors (TCEs) specifically recognizing a peptide-major histocompatibility complex (pMHC) comprising HLA-C*01:02 presenting a TP53 R273H-derived 9-mer peptide, and to methods for identifying and optimizing such TCEs.

In one aspect, the invention provides methods for identifying and optimizing TCEs by generating a plurality of candidate optimized T cell receptor (TCR) sequences, incorporating the optimized TCR sequences into TCE constructs comprising a TCR-derived binding domain, an anti-CD3 binding domain, and an Fc domain configured for heterodimerization, and evaluating the candidate TCE constructs based on binding to the pMHC, T cell activation and cytotoxicity, and reduced off-target reactivity. The evaluation may include cell-based pMHC binding assays, functional activation assays, and cytotoxicity assessments.

In another aspect, the invention provides engineered TCEs identified by the disclosed methods. The engineered TCEs may be encoded by nucleic acids or provided as polypeptides, and may be incorporated into vectors or expression cassettes comprising regulatory elements operably linked to the nucleic acids. The TCE constructs may comprise various domain arrangements, including different orders of the TCR-derived binding domain, the anti-CD3 binding domain, and the Fc domain, as well as Fc domains engineered with knob-into-hole heterodimerization configurations.

The engineered TCEs disclosed herein selectively recognize the pMHC comprising HLA-C*01:02 presenting the TP53 R273H-derived 9-mer peptide and are capable of engaging T cells to induce antigen-specific functional responses, thereby providing compositions and methods useful for the development of pMHC-targeted T cell engager technologies.

These and other advantages of the present invention will undoubtedly become apparent to those of ordinary skill in the art after reading the following detailed description of the preferred embodiments, illustrated in the various drawing figures.

### BRIEF DESCRIPTION OF THE DRAWING

The patent or application file contains at least one drawing executed in color. Copies of this patent or patent application publication with color drawing(s) will be provided by the Office upon request and payment of the necessary fee.

The accompanying drawings, which are incorporated in and form a part of this specification, illustrate embodiments of the invention and, together with the description, serve to explain the principles of the invention.

FIG. 1 is a flowchart illustrating the principle of a method for identifying and optimizing an engineered T cell engager receptor (TCE) specifically recognizing a peptide-major histocompatibility complex (pMHC) comprising HLA-C*01:02 presenting a TP53 R273H-derived 9-mer peptide in accordance with an exemplary embodiment of the present invention;

FIG. 2 shows the schematic representation of four representative TCE constructs, designated TCE002A, TCE002B, TCE002C, and TCE002D in accordance with an exemplary embodiment of the present invention;

FIG. 3 is a graph illustrating TCE-mediated cell killing activity against engineered tumor cells expressing selected HLA alleles and pulsed with the TP53 R273H-derived 25-mer peptide in accordance with an exemplary embodiment of the present invention;

FIG. 4A illustrates a panel of overlapping truncated peptides derived from a TP53 R273H 25-mer peptide, designed to map the minimal epitope presented by HLA-C*01:02 and recognized by TCE002 in accordance with an exemplary embodiment of the present invention;

FIG. 4B is a graph showing TCE002-mediated T cell cytotoxicity against tumor cells expressing HLA-C*01:02 and pulsed with the TP53 R273H-derived peptides in accordance with an exemplary embodiment of the present invention;

FIG. 5 is a graph illustrating dose-dependent binding of candidate TCE constructs (TCE002A, TCE002B, TCE002C, and TCE002D) to CD3-expressing cells, as measured by flow cytometry, in comparison with control constructs comprising TCR-Fc, and CD3-Fc;

FIG. 6A is a graph showing in-vitro binding efficiency via bio-layer interferometry (BLI) between TCE002A, TCE002B, TCE002C, and TCE002D to pMHC comprising HLA-C*01:02 presenting a TP53 R273H-derived 9-mer peptide;

FIG. 6B illustrates on-cell binding of TCE002A, TCE002B, TCE002C, and TCE002D to cells expressing the pMHC comprising HLA-C*01:02 presenting the TP53 R273H-derived 9-mer peptide via flow cytometry;

FIG. 7A is a graph showing dose-dependent T cell activation induced by candidate TCE constructs via CD69 expression on T cells following co-culture with tumor cells presenting a TP53 R273H/HLA-C*01:02 peptide-major histocompatibility complex;

FIG. 7B is a graph showing intracellular perforin expression in T cells activated by candidate TCE constructs in a dose-dependent manner upon engagement of the TP53 R273H/HLA-C*01:02 peptide-major histocompatibility complex;

FIG. 7C is a graph showing intracellular granzyme B expression in T cells following activation mediated by candidate TCE constructs targeting the TP53 R273H/HLA-C*01:02 peptide-major histocompatibility complex;

FIG. 7D is a graph showing interferon-gamma (IFN-γ) secretion by T cells as a function of candidate TCE concentration during co-culture with tumor cells presenting the TP53 R273H/HLA-C*01:02 peptide-major histocompatibility complex;

FIG. 8 shows representative microscopic images illustrating candidate TCE-mediated tumor cell cytotoxicity following co-culture of T cells with tumor cells presenting the TP53 R273H/HLA-C*01:02 peptide-major histocompatibility complex at different time points;

FIG. 9 is a graph showing T-cell dependent cytolysis of tumor cells expressing pHLA-C*01:02/TP53R273H and TCE002C when co-culturing with T cells from different donors;

FIG. 10A is a graph showing T-cell dependent cytotoxicity of TCE002C on SW620 cells expressing HLA-C*01:02 and peptides representing different TP53 R273 variants, including R273 wt, R273H, R273C, R273L, R273G, R273P, and R273S in accordance with an exemplary embodiment of the present invention;

FIG. 10B is a graph illustrating in-cell binding evaluation of TCE002C against SW620 expressing HLA-C*01:02 and peptides representing different TP53 R273 variants, including R273 wt, R273H, R273C, R273L, R273G, R273P, and R273S;

FIG. 11 is a graph showing TCE002C specificity when co-culturing with T cells and tumor cells expressing pHLA-C*01:02 with different off-target candidates naturally found in human cells;

FIG. 12A is a graph showing TCE002C-mediated T cell cytotoxicity against HLA-C*01:02-positive cells pulsed with a TP53 R273H 9-mer (H5) peptide comprising an alanine substitution at position S1 (S1A);

FIG. 12B is a graph showing TCE002C-mediated T cell cytotoxicity against HLA-C*01:02-positive cells pulsed with a TP53 R273H 9-mer (H5) peptide comprising an alanine substitution at position F2 (F2A);

FIG. 12C is a graph showing TCE002C-mediated T cell cytotoxicity against HLA-C*01:02-positive cells pulsed with a TP53 R273H 9-mer (H5) peptide comprising an alanine substitution at position E3 (E3A);

FIG. 12D is a graph showing TCE002C-mediated T cell cytotoxicity against HLA-C*01:02-positive cells pulsed with a TP53 R273H 9-mer (H5) peptide comprising an alanine substitution at position V4 (V4A);

FIG. 12E is a graph showing TCE002C-mediated T cell cytotoxicity against HLA-C*01:02-positive cells pulsed with a TP53 R273H 9-mer (H5) peptide comprising an alanine substitution at position H5 (H5A);

FIG. 12F is a graph showing TCE002C-mediated T cell cytotoxicity against HLA-C*01:02-positive cells pulsed with a TP53 R273H 9-mer (H5) peptide comprising an alanine substitution at position V6 (V6A);

FIG. 12G is a graph showing TCE002C-mediated T cell cytotoxicity against HLA-C*01:02-positive cells pulsed with a TP53 R273H 9-mer (H5) peptide comprising an alanine substitution at position C7 (C7A);

FIG. 12H is a graph showing TCE002C-mediated T cell cytotoxicity against HLA-C*01:02-positive cells pulsed with a TP53 R273H 9-mer (H5) peptide comprising an alanine substitution at position C9 (C9A);

FIG. 13A is a graph showing TCE002C-mediated T cell cytotoxicity against HLA-C*01:02-positive cells pulsed with a TP53 R273H 9-mer (H6) peptide comprising an alanine substitution at position N1 (N1A);

FIG. 13B is a graph showing TCE002C-mediated T cell cytotoxicity against HLA-C*01:02-positive cells pulsed with a TP53 R273H 9-mer (H6) peptide comprising an alanine substitution at position S2 (S2A);

FIG. 13C is a graph showing TCE002C-mediated T cell cytotoxicity against HLA-C*01:02-positive cells pulsed with a TP53 R273H 9-mer (H6) peptide comprising an alanine substitution at position F3 (F3A);

FIG. 13D is a graph showing TCE002C-mediated T cell cytotoxicity against HLA-C*01:02-positive cells pulsed with a TP53 R273H 9-mer (H6) peptide comprising an alanine substitution at position E4 (E4A);

FIG. 13E is a graph showing TCE002C-mediated T cell cytotoxicity against HLA-C*01:02-positive cells pulsed with a TP53 R273H 9-mer (H6) peptide comprising an alanine substitution at position V5 (V5A);

FIG. 13F is a graph showing TCE002C-mediated T cell cytotoxicity against HLA-C*01:02-positive cells pulsed with a TP53 R273H 9-mer (H6) peptide comprising an alanine substitution at position H6 (H6A);

FIG. 13G is a graph showing TCE002C-mediated T cell cytotoxicity against HLA-C*01:02-positive cells pulsed with a TP53 R273H 9-mer (H6) peptide comprising an alanine substitution at position V7 (V7A);

FIG. 13H is a graph showing TCE002C-mediated T cell cytotoxicity against HLA-C*01:02-positive cells pulsed with a TP53 R273H 9-mer (H6) peptide comprising an alanine substitution at position C8 (C8A);

FIG. 14A is a graph showing BLI binding responses of TCE002C and representative optimized TCE002C-derived variants to a pHLA-C*01:02/TP53 R273H 9-mer complex;

FIG. 14B is a graph showing flow cytometric quantification of on-cell binding of TCE002C and representative optimized variants to cells presenting pHLA-C*01:02/TP53 R273H 9-mer complex;

FIG. 14C is a graph showing representative flow cytometry histograms of anti-Fc staining corresponding to binding of TCE002C and representative optimized variants in the on-cell binding assay;

FIG. 14D is a graph showing comparative on-cell binding of a representative optimized variant to engineered cells presenting pHLA-C*01:02/TP53 R273H versus engineered CD3-positive cells;

FIG. 15A is a graph illustrating dose-dependent T cell-mediated cytotoxicity induced by candidate and optimized TCE constructs;

FIG. 15B is a graph illustrating concentration-dependent IFN-γ secretion by T cells in TCE-mediated co-culture assays corresponding to the candidate and optimized TCE constructs;

FIG. 16A is a graph showing dose-dependent T cell-mediated cytotoxicity of TCE002.5.4.1C against HLA-C*01:02-positive target cells pulsed with TP53 R273H-derived 9-mer peptide variants;

FIG. 16B is a set of flow cytometry histograms illustrating on-cell binding of TCE002.5.4.1C to HLA-C*01:02-positive target cells pulsed with TP53 R273H-derived 9-mer peptide variants;

FIG. 17A is a graph showing dose-dependent T cell-mediated cytotoxicity induced by TCE002.5.4.1C in co-culture assays using target cells presenting a pMHC comprising HLA-C01:02 and the TP53 R273H-derived 9-mer peptide, compared with a panel of off-target candidate peptides;

FIG. 17B is a graph showing cell killing efficiency of TCE002.5.4.1C in co-culture assays using engineered target cells expressing different HLA class I alleles and pulsed with the TP53 R273H-derived 9-mer peptide;

FIG. 17C is a graph showing the effect of peptide pulsing dose on TCE002.5.4.1C-mediated cytotoxicity using HLA-C01:02-positive target cells pulsed with the TP53 R273H-derived 9-mer peptide (H5), a corresponding TP53 wild-type 9-mer peptide, or a TP53 R273C variant 9-mer peptide;

FIG. 18A is a graph illustrating T cell-dependent cytotoxicity mediated by TCE002.5.4.1C against HLA-C*01:02-positive target cells pulsed with the TP53 R273H (H5) peptide and the S1A alanine-substituted variant thereof;

FIG. 18B is a graph illustrating T cell-dependent cytotoxicity mediated by TCE002.5.4.1C against HLA-C*01:02-positive target cells pulsed with the TP53 R273H (H5) peptide and the F2A alanine-substituted variant thereof;

FIG. 18C is a graph illustrating T cell-dependent cytotoxicity mediated by TCE002.5.4.1C against HLA-C*01:02-positive target cells pulsed with the TP53 R273H (H5) peptide and the E3A alanine-substituted variant thereof;

FIG. 18D is a graph illustrating T cell-dependent cytotoxicity mediated by TCE002.5.4.1C against HLA-C*01:02-positive target cells pulsed with the TP53 R273H (H5) peptide and the V4A alanine-substituted variant thereof;

FIG. 18E is a graph illustrating T cell-dependent cytotoxicity mediated by TCE002.5.4.1C against HLA-C*01:02-positive target cells pulsed with the TP53 R273H (H5) peptide and the H5A alanine-substituted variant thereof;

FIG. 18F is a graph illustrating T cell-dependent cytotoxicity mediated by TCE002.5.4.1C against HLA-C*01:02-positive target cells pulsed with the TP53 R273H (H5) peptide and the V6A alanine-substituted variant thereof;

FIG. 18G is a graph illustrating T cell-dependent cytotoxicity mediated by TCE002.5.4.1C against HLA-C*01:02-positive target cells pulsed with the TP53 R273H (H5) peptide and the C7A alanine-substituted variant thereof;

FIG. 18H is a graph illustrating T cell-dependent cytotoxicity mediated by TCE002.5.4.1C against HLA-C*01:02-positive target cells pulsed with the TP53 R273H (H5) peptide and the C9A alanine-substituted variant thereof;

FIG. 19 is a graph illustrating T cell-mediated cytotoxic activity induced by TCE constructs across different human cell types, including tumor cells presenting HLA-C*01:02 in complex with a TP53 R273H-derived peptide and non-tumor control cells lacking the target pMHC;

FIG. 20 is a graph illustrating comparative T cell-mediated cytotoxicity elicited by TCE002C and an optimized TCE variant on tumor cells expressing HLA-C*01:02 and endogenously harboring TP53 R273 mutations in heterozygous or compound heterozygous form;

FIG. 21 is a graph illustrating the functional stability assessment of optimized TCE constructs following incubation in serum over defined time intervals, as determined by normalized interferon-gamma (IFN-γ) release from activated T cells relative to the initial time point;

FIG. 22 illustrates representative fluorescence and brightfield images of multicellular 3D spheroids comprising stromal cells, endothelial cells, and tumor cells, captured at multiple time points following co-culture with T cells in the presence or absence of TCE002C;

FIG. 23A is a graph showing dose-dependent T cell-mediated cytotoxicity against 3D tumor spheroids induced by TCE002C, as measured by a luminescent cell viability assay;

FIG. 23B is a graph illustrating concentration-dependent IFN-γ secretion from T cells co-cultured with 3D tumor spheroids in the presence of TCE002C;

FIG. 24 illustrates representative fluorescence and brightfield images of three-dimensional tumor spheroids comprising SW620 cells expressing HLA-C*01:02 presenting a TP53 R273H 9-mer peptide, co-cultured with T cells and treated with TCE002.5.4.1C alone or in combination with chemotherapeutic agents over time;

FIG. 25A is a graph showing dose-dependent TCE002.5.4.1C-mediated T cell cytotoxicity in a three-dimensional tumor spheroid model, in the absence or presence of irinotecan or oxaliplatin;

FIG. 25B is a graph illustrating concentration-dependent IFN-γ release from T cells in three-dimensional tumor spheroid co-cultures treated with TCE002.5.4.1C alone or in combination with irinotecan or oxaliplatin;

FIG. 26 is a series of fluorescence and brightfield microscopy images showing three-dimensional tumor spheroids comprising endothelial cells, adipose-derived stem cells, and colorectal cancer cells expressing HLA-C*01:02 and endogenously harboring a TP53 R273H mutation, following co-culture with T cells and TCE constructs at 0, 24, and 48 hours; and

FIG. 27 is a graph illustrating dose-dependent T cell-mediated cytotoxicity of TCE002.5.4.1C against three-dimensional colorectal cancer spheroids carrying an endogenous TP53 R273H mutation, as measured by a luminescent cell viability assay.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

Reference will now be made in detail to the preferred embodiments of the invention, examples of which are illustrated in the accompanying drawings. While the invention will be described in conjunction with these preferred embodiments, it should be understood that they are not intended to limit the invention. On the contrary, the invention is intended to cover alternatives, modifications, and equivalents that may be included within the spirit and scope of the invention as defined by the appended claims. Furthermore, in the following detailed description of the present invention, numerous specific details are set forth to provide a thorough understanding. However, it will be obvious to one of ordinary skills in the art that the present invention may be practiced without these specific details. In other instances, well-known methods, procedures, components, and circuits have not been described in detail so as not to unnecessarily obscure aspects of the present invention.

It should be noted that the terms “first”, “second” and the like in the specification and claims of the present invention are used to distinguish similar objects and are not necessarily indicative of a specific order. It is to be understood that the terms are interchangeable, where appropriate, allowing the embodiments of the invention described herein to be implemented in a sequence other than those illustrated or described. Additionally, the terms “comprises” and “comprising”, as well as “the” and “these”, are intended to cover a non-exclusive inclusion. For example, a process, method, system, product, or device that comprises a series of steps or units is not necessarily limited to those explicitly listed and may include other steps or units not explicitly mentioned or inherent to such processes, methods, products, or devices.

Unless otherwise defined, all technical and scientific terms used herein have the same meaning as commonly understood by one of ordinary skill in the art to which this invention belongs. The following definitions are provided for clarity and consistency of interpretation.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

By “binding” is meant attaching by a covalent bond or a non-covalent bond. Non-covalent bonds include those formed by van der Waals forces, hydrogen bonds, ionic bonds, entrapment or physical encapsulation, absorption, adsorption, and/or other intermolecular forces. Binding can be effectuated by any useful means, such as by enzymatic binding (e.g., enzymatic ligation) or by chemical binding (e.g., chemical ligation).

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

The term “conservative amino acid substitution” refers to the replacement of an amino acid residue with another amino acid residue having similar physicochemical properties, such that the substitution is unlikely to substantially affect the biological activity, structure, or function of the resulting polypeptide. Conservative substitution schemes providing functionally similar amino acids are well known in the art and have been described in standard references, including, for example, Creighton, Proteins: Structures and Molecular Properties (W. H. Freeman & Co., 2nd ed., 1993). By way of non-limiting example, conservative amino acid substitutions may be made within the following groups of amino acids: Alanine (A) and Glycine (G); Aspartic acid (D) and Glutamic acid (E); Asparagine (N) and Glutamine (Q); Arginine (R) and Lysine (K); Isoleucine (I), Leucine (L), Methionine (M), and Valine (V); Phenylalanine (F), Tyrosine (Y), and Tryptophan (W); Serine(S) and Threonine (T); and Cysteine (C) and Methionine (M). Conservative amino acid substitutions as described herein may be applied to the polypeptides disclosed in this application, provided that such substitutions do not materially alter the ability of the engineered T cell engager receptor (TCE) to specifically recognize the peptide-major histocompatibility complex as defined in the claims.

The term “T-cell receptor (TCR)” meaning is a protein complex found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide, and many antigen peptides are recognized by the same TCR.

A “vector” or “expression cassette” refers to a nucleic acid construct comprising a TCE-encoding nucleic acid operably linked to one or more regulatory elements, including promoters, enhancers, transcription termination signals, and polyadenylation signals, suitable for expression in a host cell.

The term “T-cell receptor engager (TCE)” refers to the bispecific engager capable of recognizing both human cluster of differentiation (CD3) complex and human tetramer comprises of a major histocompatibility complex (MHC) class I.

The term “specifically binds” means that the binding is selective for the target and can be discriminated from unwanted or non-specific interactions.

The term “human leukocyte antigen (HLA)” refers to the expression product of the Major Histocompatibility Complex (MHC) in humans.

The term “Peripheral blood mononuclear cells (PBMC)” refers to the group of mononuclear cells retrievable from a subject's blood, including T cells.

As used herein, the term “peptide” refers to a polypeptide comprising a plurality of amino acid residues. In a living organism, the immune system functions to recognize and eliminate materials that are not derived from the organism itself. Certain peptides, including peptides derived from exogenous sources or from endogenous genetic alterations, may be immunogenic and capable of eliciting an immune response. During oncogenesis, alterations in a subject's genetic material, including somatic mutations, can give rise to neoantigenic peptides. Such neoantigenic peptides may be processed and presented on the surface of tumor cells by human leukocyte antigen (HLA) molecules, including HLA class I (HLA-I) or HLA class II (HLA-II) molecules. Presentation of these peptides may result in recognition by T cells, including CD8⁺ T cells and/or CD4⁺ T cells, thereby initiating an immune response through coordinated intracellular and intercellular signaling pathways. Neoantigenic peptides typically contain at least one mutated amino acid residue and may vary in length depending on the presenting HLA molecule. By way of example, peptides presented by HLA-I molecules are often about 8 to 11 amino acids in length, while peptides presented by HLA-II molecules may be longer, such as approximately 25 amino acids, although peptides of other lengths are also contemplated.

The term “mutant peptide” or “MT” refers to a peptide comprising at least one amino acid residue that differs from the corresponding amino acid residue present in the wild-type sequence and that is selectively present in tumor tissue but not in normal tissue.

As used herein, the term “wild-type” or “WT”, and grammatical equivalents thereof, refers to an amino acid sequence or a nucleotide sequence representing the typical or non-mutated form found in normal cells, including naturally occurring allelic variations, and excluding cancer-associated modifications.

The phrase “immunogenic neoantigens” refers to peptides, including long peptides, that comprise one or more shared or recurrent somatic mutations and that are capable of eliciting a T cell-mediated immune response directed against the mutant peptide.

The term “effective amount” refers to an amount of a compound, composition, or molecule sufficient to achieve a desired biological or therapeutic effect, including but not limited to activation of T cells, induction of cytotoxic activity, or inhibition of tumor cell growth.

Furthermore, as used herein, the term “GStARG-Et™” refers to Gene Solutions' targeted Antigen Receptor Gene-Engager therapy, which is a commercial designation under which a collection of engineered T cell engager receptor (TCE) molecules, including multiple sequence variants and molecular configurations, is commercially offered.

### Chimeric Bispecific T Cell Engager Receptor Constructs.

### General Description of TCE Constructs

Provided herein are chimeric bispecific T cell engager receptor (TCE) constructs comprising two or more recombinant polypeptide domains configured to simultaneously engage a peptide-major histocompatibility complex (pMHC) on a target cell and a CD3 complex on a T cell. In accordance with the present disclosure, the TCE constructs are configured as single-chain recombinant molecules and are suitable for use in immune redirection strategies involving T cell activation and target cell cytotoxicity. The TCE constructs disclosed herein comprise a T cell receptor-derived binding domain, an anti-CD3 binding domain, and an Fc domain, arranged in defined domain orders as described in the claims. The TCE constructs may be provided as nucleic acids encoding such polypeptides or as the polypeptides themselves.

### Exemplary TCE Constructs

In some embodiments, the TCE constructs comprises: a TCR-derived binding domain capable of recognizing a pMHC comprising HLA-C*01:02 presenting a TP53 R273H-derived 9-mer peptide; an anti-CD3 binding domain capable of binding a CD3 complex expressed on a T cell; and an Fc domain configured to enhance molecular stability and serum persistence.

In some embodiments, the TCR-derived binding domain is positioned at the N-terminus of the TCE, and the Fc domain is positioned C-terminal to the anti-CD3 binding domain.

In other embodiments, the TCR-derived binding domain is positioned at the N-terminus, the Fc domain is positioned downstream thereof, and the anti-CD3 binding domain is positioned at the C-terminus of the TCE.

In all such embodiments, the TCR-derived binding domain comprises variable regions derived from T cell receptor α and β chains. In some embodiments, transmembrane regions of the native TCR chains are absent. In further embodiments, constant regions of the TCR chains are removed, and the variable regions are incorporated into a single-chain TCR (sTCR) or stapled single-chain TCR (spTCR) format.

### Fragment Crystallizable (Fc) Region

Functional role of the Fc domain: the Fc domain contributes to the pharmacokinetic properties of the TCE, including serum half-life and molecular stability. Fc regions derived from immunoglobulins exhibit pH-dependent recycling via Fc receptors, thereby reducing degradation and prolonging systemic persistence. In the context of the TCE constructs disclosed herein, fusion of the Fc domain to the TCR-derived binding domain and the anti-CD3 binding domain enhances circulating stability, allows sustained biological activity, and may reduce dosing frequency.

### Fc Isotypes

In some embodiments, the Fc domain is derived from, or based on, an immunoglobulin selected from the group consisting of IgG, IgA, IgM, IgE, and IgD. In preferred embodiments consistent with the claims, the Fc domain is derived from IgG.

### Fc Mutations

In some embodiments, the Fc domain comprises one or more amino acid substitutions configured to modulate dimerization, stability, or effector function. Such substitutions may include mutations at positions corresponding to one or more of the following residues: L234, L235, D265, R292, N297, V302, P329, Y349, S354, T366, L368, and Y407, according to standard immunoglobulin numbering schemes. Substitutions may be present at a single residue or at a combination of residues and may be configured to promote heterodimerization, reduce Fc receptor binding, or attenuate immune effector functions, consistent with the intended function of the TCE.

### Linkers

In some embodiments, domains within the TCE architecture are linked by one or more flexible peptide linkers. Such linkers may comprise glycine- and serine-rich sequences configured to provide conformational flexibility and proper spatial orientation of the linked domains. In particular embodiments, the Fc polypeptide fragments are linked by a glycine-serine linker, including linkers comprising repeated G4S motifs, as described in the claims.

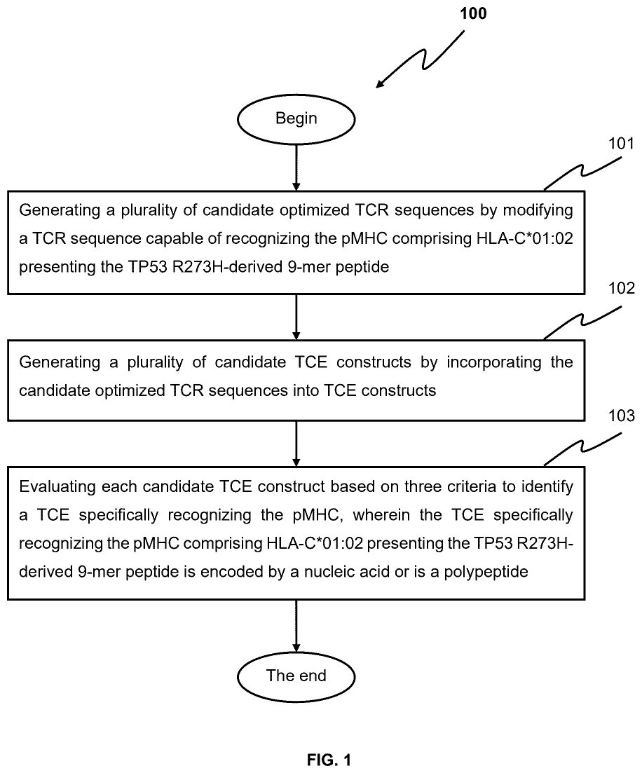

One embodiment of the invention is now described with reference to FIG. 1. FIG. 1 illustrated a method for identifying and optimizing an engineered T cell engager receptor (TCE) specifically recognizing a peptide-major histocompatibility complex (pMHC) comprising HLA-C*01:02 presenting a TP53 R273H-derived 9-mer peptide 100 (“method 100”) based on the above principle in accordance with an exemplary embodiment of the present invention. In particular, method 100 includes the following three steps: step 101, step 102, and step 103.

At step 101, generating a plurality of candidate optimized T cell receptor (TCR) sequences by modifying a TCR sequence capable of recognizing the pMHC comprising HLA-C*01:02 presenting the TP53 R273H-derived 9-mer peptide.

According to the preferred embodiment of the present invention, the TCR sequence is selected from the group consisting of SEQ ID NOs: 1-4. The TCR sequences identified and reported in U.S. patent Ser. No. 12/442,046.

According to the embodiment of the present invention, the plurality of candidate optimized TCR sequences comprises amino acid sequences selected from the group consisting of SEQ ID NOs: 5-31.

According to the embodiment of the present invention, the TP53 R273H-derived 9-mer peptide is selected from the group consisting of SEQ ID NOs: 32-33, or an alanine-substituted variant thereof;
wherein the alanine-substituted variant comprises a single alanine substitution at any one amino acid position of the TP53 R273H-derived 9-mer peptide sequence set forth in SEQ ID NO: 32 or SEQ ID NO: 33;
the single alanine-substituted variant of the peptide sequence set forth in SEQ ID NO: 32 is selected from the group consisting of SEQ ID NOs: 34-41; and
the single alanine-substituted variant of the peptide sequence set forth in SEQ ID NO: 33 is selected from the group consisting of SEQ ID NOs: 42-49.

At step 102, generating a plurality of candidate TCE constructs by incorporating the candidate optimized TCR sequences generated in step 101 into TCE constructs, wherein each candidate TCE construct comprises:
(b1) a TCR-derived binding domain comprising the candidate optimized TCR sequence in a single-chain TCR (sTCR) format or a stapled single-chain TCR (spTCR) format, wherein the TCR-derived binding domain comprises a variable α chain region (α-TCR) and a variable β chain region (β-TCR);
(b2) an anti-CD3 binding domain capable of binding to a CD3 complex expressed on a T cell, wherein the anti-CD3 binding domain comprises a variable heavy chain region (α-anti-CD3) and a variable light chain region (β-anti-CD3) arranged in a single-chain variable fragment (scFv) format or a stapled single-chain variable fragment (spFv) format; and
(b3) an Fc domain comprises a first Fc polypeptide fragment and a second Fc polypeptide fragment configured in a knob-into-hole heterodimerization arrangement, in which an N-terminus of the second Fc polypeptide fragment is linked to a C-terminus of the first Fc polypeptide fragment by a peptide linker consisting of a 30-amino-acid glycine-serine sequence comprising five repeats of a G4S pentapeptide.

According to the embodiment of the present invention, each candidate TCE construct comprises the domains arranged, from N-terminus to C-terminus, in an order selected from the group consisting of:
the TCR-derived binding domain, the anti-CD3 binding domain, and the Fc domain; or
the TCR-derived binding domain, the Fc domain, and the anti-CD3 binding domain.

According to the preferred embodiment of the present invention, each candidate TCE construct comprises, from N-terminus to C-terminus:
the T cell receptor-derived binding domain in the sTCR format or the spTCR format;
the anti-CD3 binding domain in the scFv format; and
the Fc domain.

According to the preferred embodiment of the present invention, the domain order of each candidate TCE construct, from N-terminus to C-terminus, is selected from the group consisting of:
(i) α-TCR, β-TCR, α-anti-CD3, β-anti-CD3, the first Fc polypeptide fragment comprising a knob mutation, and the second Fc polypeptide fragment comprising a hole mutation;
(ii) α-TCR, β-TCR, α-anti-CD3, β-anti-CD3, the first Fc polypeptide fragment comprising a hole mutation, and the second Fc polypeptide fragment comprising a knob mutation;
(iii) β-TCR, α-TCR, α-anti-CD3, β-anti-CD3, the first Fc polypeptide fragment comprising a knob mutation, and the second Fc polypeptide fragment comprising a hole mutation;
(iv) β-TCR, α-TCR, α-anti-CD3, β-anti-CD3, the first Fc polypeptide fragment comprising a hole mutation, and the second Fc polypeptide fragment comprising a knob mutation;
(v) α-TCR, β-TCR, β-anti-CD3, α-anti-CD3, the first Fc polypeptide fragment comprising a knob mutation, and the second Fc polypeptide fragment comprising a hole mutation;
(vi) α-TCR, β-TCR, β-anti-CD3, α-anti-CD3, the first Fc polypeptide fragment comprising a hole mutation, and the second Fc polypeptide fragment comprising a knob mutation;
(vii) β-TCR, α-TCR, β-anti-CD3, α-anti-CD3, the first Fc polypeptide fragment comprising a knob mutation, and the second Fc polypeptide fragment comprising a hole mutation; and

(viii) β-TCR, α-TCR, β-anti-CD3, α-anti-CD3, the first Fc polypeptide fragment comprising a hole mutation, and the second Fc polypeptide fragment comprising a knob mutation.

According to the preferred embodiment of the present invention, each candidate TCE construct comprises, from N-terminus to C-terminus:
the T cell receptor-derived binding domain in the sTCR format or the spTCR format;
the Fc domain; and
the anti-CD3 binding domain in the scFv format.

According to the preferred embodiment of the present invention, the domain order of each candidate TCE construct, from N-terminus to C-terminus, is selected from the group consisting of:
(i′) α-TCR, β-TCR, the first Fc polypeptide fragment comprising a knob mutation, the second Fc polypeptide fragment comprising a hole mutation, α-anti-CD3, and β-anti-CD3;
(ii′) α-TCR, β-TCR, the first Fc polypeptide fragment comprising a hole mutation, the second Fc polypeptide fragment comprising a knob mutation, α-anti-CD3, and β-anti-CD3;
(iii′) β-TCR, α-TCR, the first Fc polypeptide fragment comprising a knob mutation, the second Fc polypeptide fragment comprising a hole mutation, α-anti-CD3, and β-anti-CD3;
(iv′) β-TCR, α-TCR, the first Fc polypeptide fragment comprising a hole mutation, the second Fc polypeptide fragment comprising a knob mutation, α-anti-CD3, and β-anti-CD3;
(v′) α-TCR, β-TCR, the first Fc polypeptide fragment comprising a knob mutation, the second Fc polypeptide fragment comprising a hole mutation, β-anti-CD3, and α-anti-CD3;
(vi′) α-TCR, β-TCR, the first Fc polypeptide fragment comprising a hole mutation, the second Fc polypeptide fragment comprising a knob mutation, β-anti-CD3, and α-anti-CD3;
(vii′) β-TCR, α-TCR, the first Fc polypeptide fragment comprising a knob mutation, the second Fc polypeptide fragment comprising a hole mutation, β-anti-CD3, and α-anti-CD3; and
(viii′) β-TCR, α-TCR, the first Fc polypeptide fragment comprising a hole mutation, the second Fc polypeptide fragment comprising a knob mutation, β-anti-CD3, and α-anti-CD3.

Finally, at step 103, evaluating each candidate TCE construct based on three criteria to identify a TCE specifically recognizing the peptide-major histocompatibility complex (pMHC), wherein the TCE specifically recognizing the pMHC comprising HLA-C*01:02 presenting the TP53 R273H-derived 9-mer peptide is encoded by a nucleic acid or is a polypeptide.

According to the embodiment of the present invention, the three criteria comprise:
(c1) evaluating binding of the candidate TCE constructs to the pMHC comprising HLA-C*01:02 presenting the TP53 R273H-derived 9-mer peptide, wherein evaluating binding comprises:
   (A) providing cells expressing HLA-C01:02;
   (B) pulsing the cells with the TP53 R273H-derived 9-mer peptide to form a pHLA-C01:02/TP53 R273H peptide complex;
   (C) incubating the pHLA-C01:02/TP53 R273H peptide-expressing cells with the candidate TCE constructs;
   (D) stabilizing interactions between the candidate TCE constructs and the pHLA complexes by cross-linking with formaldehyde at a final concentration of 1% (v/v);
   (E) quenching the cross-linking reaction by contracting the cells with Tris-HCl at pH 8.0 at a final concentration of 0.75 M;
   (F) washing the cells to remove unbound the candidate TCE constructs and reagents;
   (G) detecting TCE-bound cells using an Fc-specific detection reagent; and
   (H) quantifying TCE binding to the pHLA-expressing cells by flow cytometry;
(c2) evaluating functional activation and T cell-mediated cytotoxicity induced by the candidate TCE constructs by co-culturing immune effector cells with antigen-presenting tumor cells in the presence of the candidate TCE constructs;
   wherein T cell activation is evaluated by detecting expression of one or more markers selected from the group consisting of CD69, perforin, and granzyme B; and
   wherein T cell-mediated cytotoxicity is evaluated by measuring interferon-gamma (IFN-γ) release and/or assessing tumor cell killing; and
(c3) evaluating reduced off-target reactivity relative to binding to the pMHC comprising HLA-C01:02 presenting the TP53 R273H-derived 9-mer peptide.

According to the embodiment of the present invention, the cells expressing the pMHC comprising HLA-C*01:02 presenting the TP53 R273H-derived 9-mer peptide comprise antigen-presenting tumor cells; wherein the antigen-presenting tumor cells are derived from a tumor cell line selected from the group consisting of SW620 colorectal carcinoma cells, H520 lung squamous carcinoma cells, and MDA-MB-157 breast carcinoma cells.

According to the embodiment of the present invention, the TCE specifically recognizing the pMHC comprising HLA-C*01:02 presenting the TP53 R273H-derived 9-mer peptide is encoded by the nucleic acid comprising a nucleotide sequence selected from the group consisting of SEQ ID NOs: 56-61;
wherein the nucleic acid is DNA or RNA, the DNA is selected from the group consisting of circular plasmid DNA, linear double-stranded DNA, single-stranded DNA, or a chimeric nucleic acid comprising DNA and RNA, and the RNA is messenger RNA (mRNA).

According to the embodiment of the present invention, the TCE specifically recognizing the pMHC comprising HLA-C*01:02 presenting the TP53 R273H-derived 9-mer peptide is the polypeptide comprising an amino acid sequence selected from the group consisting of SEQ ID NOs: 50-55.

According to the embodiment of the invention, the method 100 is applied to lung cancer, breast cancer, and colorectal cancer.

In certain embodiments, the anti-CD3 binding domain comprises an amino acid sequence selected from the group consisting of the sequences set forth in SEQ ID NOs: 120-123, and the Fc domain comprises an amino acid sequence selected from the group consisting of the sequences set forth in SEQ ID NOs: 124-125.

The present disclosure provides an engineered T cell engager receptor (TCE) specifically recognizing a peptide-major histocompatibility complex (pMHC) comprising HLA-C*01:02 presenting a TP53 R273H-derived 9-mer peptide, wherein the engineered TCE is encoded by a nucleic acid comprising a nucleotide sequence selected from the group consisting of SEQ ID NOs: 56-61, and wherein the the engineered TCE is identified by the method 100 described above.

According to the embodiment of the present invention, the nucleic acid encoding the engineered TCE is comprised within a vector or an expression cassette; wherein the vector or expression cassette comprises one or more regulatory elements operably linked to the nucleic acid; and
the regulatory elements being selected from the group consisting of a promoter, an enhancer, a transcription termination signal, a polyadenylation signal, and combinations thereof.

The present disclosure provides an engineered T cell engager receptor (TCE) specifically recognizing a peptide-major histocompatibility complex (pMHC) comprising HLA-C*01:02 presenting a TP53 R273H-derived 9-mer peptide, wherein the engineered TCE is a polypeptide comprising an amino acid sequence selected from the group consisting of SEQ ID NOs: 50-55, and wherein the engineered TCE is identified by the method 100 described above.

According to the embodiment of the present invention, a nucleic acid encoding the polypeptide comprising the engineered TCE is comprised within a vector or an expression cassette;
wherein the vector or expression cassette comprises one or more regulatory elements operably linked to the nucleic acid; and
the regulatory elements being selected from the group consisting of a promoter, an enhancer, a transcription termination signal, a polyadenylation signal, and combinations thereof.

It will be understood by those skilled in the art that features of the present disclosure, which are described herein in the context of separate embodiments for purposes of clarity, may be combined in any suitable manner in a single embodiment. Conversely, features of the disclosure described in the context of a single embodiment may also be implemented separately or in any suitable sub-combination. All combinations of the embodiments described herein are expressly contemplated and are within the scope of the present disclosure, as if each such combination were individually and explicitly described. Likewise, all sub-combinations of the various embodiments and elements thereof are expressly contemplated and disclosed herein, as if each such sub-combination were individually and explicitly set forth.

This example describes the generation, expression, and functional evaluation of engineered T cell engager receptors (TCEs) comprising a T cell receptor-derived binding domain, an anti-CD3 binding domain, and an Fc domain, as defined in the method 100.

### Definition of TCR002 and TCE002

As used herein, the term “TCR002” refers to a T cell receptor sequence comprising the amino acid sequences set forth in SEQ ID NOs: 1-4, corresponding to the T cell receptor designated TCR12.1, as previously disclosed in U.S. patent Ser. No. 12/442,046.

When the TCR002 sequence is incorporated into an engineered T cell engager receptor (TCE) architecture according to the present invention, the resulting molecule is referred to as “TCE002.”

The designation TCE002 may be further appended with alphabetical suffixes, including but not limited to TCE002A, TCE002B, TCE002C, and TCE002D, to denote distinct TCE formats, molecular constructs, domain arrangements, or structural configurations that incorporate the same underlying TCR002 sequence.

Unless otherwise expressly indicated, references to TCE002 herein encompass all engineered T cell engager receptor constructs derived from TCR002, regardless of specific format, domain order, linker composition, or structural configuration.

### (A2) Generation of the Candidate TCE Constructs

T cell receptor (TCR) α and β chain sequences capable of recognizing the pMHC comprising HLA-C*01:02 presenting a TP53 R273H-derived peptide were selected based on prior identification. The TCR sequences correspond to amino acid sequences set forth in SEQ ID NOs: 5-31, consistent with the plurality of candidate optimized TCR sequences recited in step 101.

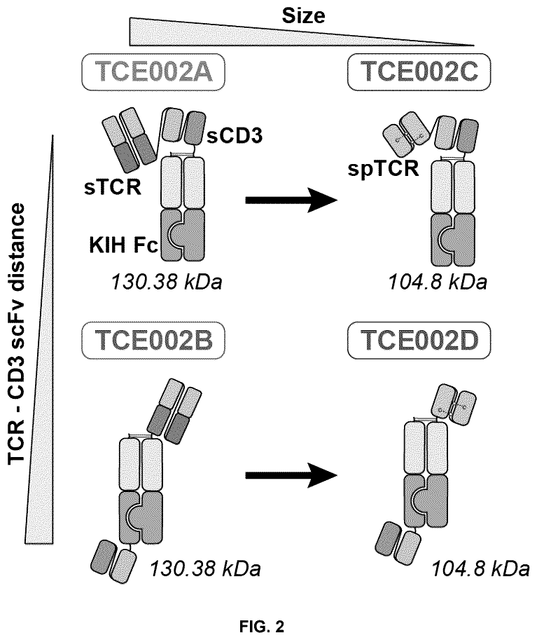

The selected T cell receptor (TCR) α and β chain variable regions were incorporated into TCE constructs to generate a plurality of candidate TCE constructs. As a result, four representative TCE constructs, designated TCE002A, TCE002B, TCE002C, and TCE002D, were generated, each differing in domain arrangement and TCR format, as schematically illustrated in FIG. 2. Each representative TCE construct is encoded by a nucleic acid sequence and comprises a corresponding polypeptide sequence, wherein the nucleic acid sequences and polypeptide sequences are selected from the SEQ ID NOs shown in Table 1 below.

TABLE 1
Representative TCE constructs and SEQ ID numbers

SEQ ID NOs

| | Encoded | Encoded |
|---|---|---|
| TCE construct | nucleic acid | polypeptide |
| TCE002A | 66 | 62 |
| TCE002B | 67 | 63 |
| TCE002C | 68 | 64 |
| TCE002D | 69 | 65 |

As shown in FIG. 2, TCE002A and TCE002B comprise sTCR format operably linked to an anti-CD3 binding domain and a knob-into-hole (KIH) Fc domain, with TCE002B differing from TCE002A by an inverted relative orientation of the anti-CD3 variable domains. TCE002C and TCE002D comprise spTCR format in place of the sTCR, while maintaining the same functional domains and Fc configuration, with TCE002D likewise differing by inversion of the anti-CD3 domain orientation. The use of the spTCR format in TCE002C and TCE002D results in a reduced molecular size relative to the sTCR-based constructs TCE002A and TCE002B, as reflected by the lower calculated molecular weight. In addition, the spTCR configuration alters the geometric spacing between the TCR-derived binding domain and the anti-CD3 binding domain, which may influence immune synapse formation and T cell activation. Such variations in inter-domain spacing and molecular architecture represent tunable engineering parameters rather than routine or predetermined design choices.
(B2) Expression and purification of candidate TCE constructs: nucleic acids encoding the candidate TCE constructs generated as described herein were cloned into mammalian expression vectors and transiently transfected into HEK293T cells. The candidate TCE polypeptides were expressed and purified from cell culture supernatants using standard affinity chromatography followed by high-performance liquid chromatography (HPLC) to obtain purified TCE constructs.
(C2) Preparation of immune effector cells: peripheral blood mononuclear cells (PBMCs) were isolated from peripheral blood samples obtained from human donors. T cells were isolated from PBMCs and cultured in AIM-V™ medium supplemented with 10% fetal bovine serum (FBS) to enrich T cells prior to use in co-culture assays.
(D2) Preparation of Target Cells and Peptide Pulsing

Tumor cell lines expressing defined HLA class I alleles were used as antigen-presenting target cells in the functional evaluation of the candidate TCE constructs. In particular embodiments, SW620 colorectal carcinoma (CRC) cells engineered to express HLA-C01:02 were employed as the primary antigen-presenting cell model for assessing TCE activity toward the peptide-major histocompatibility complex (pMHC) comprising HLA-C01:02 presenting the TP53 R273H-derived peptide.

Synthesizing a long peptide corresponding to a panel of TP53 R273H-derived neoantigenic peptide, including the TP53 R273H 25-mer peptide (SEQ ID NO: 70) listed in Table 2 below.

TABLE 2
TP53 R273H-derived peptides and corresponding SEQ
ID numbers used for evaluation of TCE activity

| TP53 R273H mutation | SEQ ID No. |
|---|---|
| TP53 R273H 25-mer peptide | 70 |
| TP53 R273H 9-mer peptide (H5) | 32 |
| TP53 R273H 9-mer peptide (H6) | 33 |
| TP53 R273H 10-mer peptide (H9) | 71 |
| TP53 R273H 10-mer peptide (H6) | 72 |
| TP53 R273H 10-mer peptide (H7) | 73 |

As used herein, the designations H5, H6, H7, and H9 refer to internal identifiers for truncated TP53 R273H-derived peptides, each representing a distinct 9-mer or 10-mer epitope spanning the TP53 R273H mutation.

### (E2) TCE-Mediated Co-Culture Assays

T cells were prepared for co-culture with the peptide-pulsed tumor cells. PBMCs were thawed in AIM-V medium containing 2% FBS, followed by isolation and expansion of T cells in the same medium for at least 8 hours.

Pulsed tumor cells were co-cultured with T cells at a defined effector-to-target (E: T) ratio of 5:1 in the presence of a candidate TCE construct. TCE constructs were evaluated at final assay concentrations ranging from approximately 10⁻⁴ nM to 10 nM.

It should be noted that TCE002A, TCE002B, TCE002C, or TCE002D was selected as a representative candidate TCE construct for functional characterization (e.g., HLA restriction and peptide mapping), while the remaining TCE002 formats or TCE format were retained as alternative constructs for comparative evaluation. Tumor cell line is selected from the group consisting of SW620 colorectal carcinoma cells, H520 lung squamous carcinoma cells, and MDA-MB-157 breast carcinoma cells.

### (F2) Evaluation of T Cell Activation and Cytotoxicity

TCE-mediated activity was assessed by two functional readouts:
tumor cell viability, measured using a luminescent cell viability assay (CellTiter-Glo 2D), to determine percent specific lysis as an indicator of T cell-mediated cytotoxicity; and
T cell activation, quantified by interferon-gamma (IFN-γ) secretion in the co-culture supernatant.

(G2) HLA Restriction Analysis: to evaluate the HLA restriction of the engineered TCE constructs, tumor cells individually expressing distinct HLA class I alleles, including HLA-A02:06, HLA-A11:01, HLA-B40:01, HLA-B54:01, HLA-C01:02, and HLA-C07:02, were employed. The tumor cells were pulsed with a TP53 R273H-derived 25-mer peptide and subsequently assessed in parallel TCE-mediated co-culture assays.

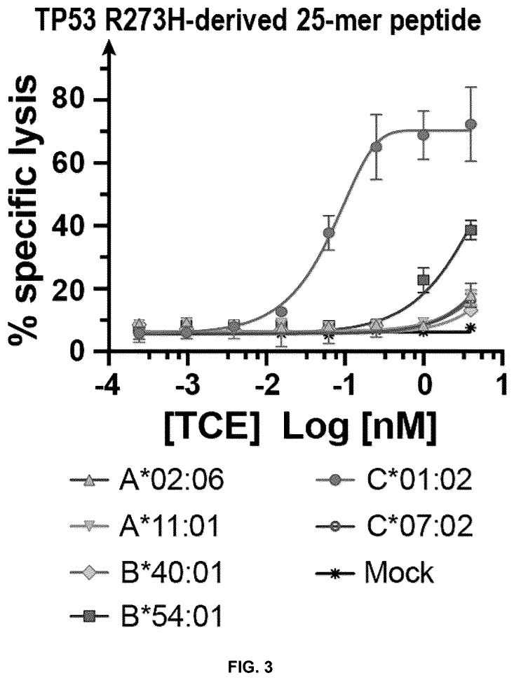

Referring to FIG. 3, TCE002A-mediated T cell cytotoxicity was evaluated using tumor cells expressing defined HLA alleles and pulsed with the TP53 R273H-derived 25-mer peptide. The results demonstrate a pronounced and dose-dependent increase in percent specific lysis when target cells expressed HLA-C*01:02, with maximal cytotoxicity reaching approximately 70-75% at TCE concentrations between approximately 0.1 and 10 nM. In contrast, tumor cells expressing other HLA alleles, including HLA-A02:06, HLA-A11:01, HLA-B40:01, and HLA-C07:02, exhibited negligible cytotoxicity across the tested TCE concentration range. Tumor cells expressing HLA-B*54:01 showed only limited cytotoxicity at higher TCE concentrations, indicating substantially reduced functional engagement relative to HLA-C*01:02. These data indicate that the TCE constructs exhibit HLA-restricted functional activity, with preferential recognition of the pMHC comprising HLA-C*01:02 presenting the TP53 R273H-derived peptide. The sharp difference in cytotoxic response between HLA-C*01:02 and other tested HLA alleles supports the specificity of the TCR-derived binding domain incorporated into the TCE constructs.

The HLA-restricted cytotoxicity profile observed in FIG. 3 prompted further investigation into the peptide specificity underlying TCE002A-mediated T cell activation. While FIG. 3 demonstrates preferential cytotoxicity toward tumor cells expressing HLA-C*01:02 and pulsed with the TP53 R273H-derived 25-mer peptide, the precise peptide determinants responsible for functional engagement by the TCE constructs were not resolved by that analysis alone. Accordingly, additional studies were conducted to delineate the minimal peptide epitope(s) capable of supporting TCE-mediated recognition and cytotoxicity in the context of HLA-C*01:02.

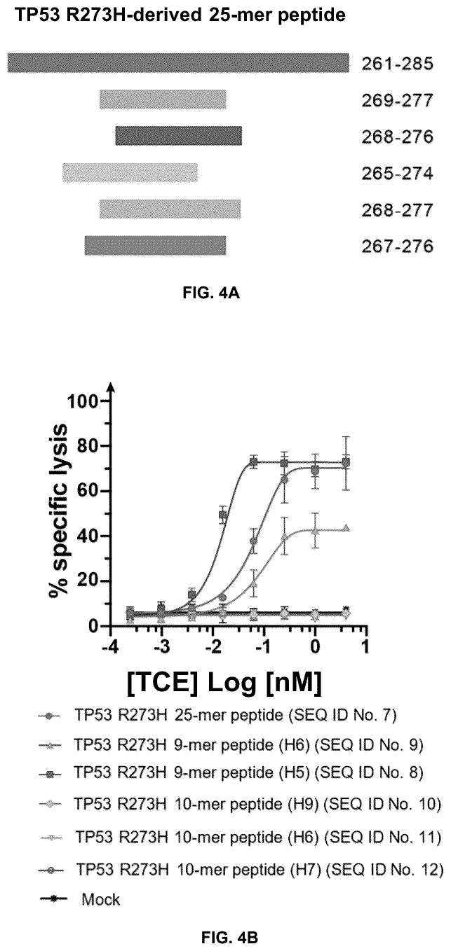

(H2) Epitope Mapping Using Truncated TP53 R273H-Derived Peptides: to identify the minimal epitope recognized by the TCE constructs, a series of truncated peptides comprising overlapping regions of a TP53 R273H-derived 25-mer peptide were synthesized as shown in FIG. 4A. SW620 target cells expressing HLA-C*01:02 were individually pulsed with each truncated peptide and evaluated in TCE-mediated cytotoxicity assays as shown in FIG. 4B.

Referring to FIG. 4A, a series of truncated peptides derived from a TP53 R273H-derived 25-mer peptide (SEQ ID NO: 70; amino acid positions 261-285) were designed to further evaluate peptide specificity and HLA-restricted recognition by the TCE constructs. The truncated peptides comprised overlapping 9-mer and 10-mer TP53 R273H-derived peptides, each retaining the histidine residue corresponding to the R273H substitution. The peptides differed in N-terminal and C-terminal boundaries relative to the mutation site, thereby enabling assessment of the influence of flanking residues on formation of the pMHC and on TCE-mediated functional activity.

Referring to FIG. 4B, TCE002-mediated T cell cytotoxicity was evaluated using tumor cells expressing HLA-C*01:02 and pulsed with the TP53 R273H-derived peptides illustrated in FIG. 4A. The results demonstrate that only a defined subset of the tested TP53 R273H-derived peptides supported robust and dose-dependent T cell-mediated cytotoxicity when engaged by TCE002. In particular, tumor cells pulsed with the TP53 R273H-derived 9-mer peptides comprising SEQ ID NO: 32 and SEQ ID NO: 72 exhibited high levels of specific lysis, reaching approximately 70-80% specific killing at nanomolar concentrations of TCE002. These peptides displayed clear sigmoidal dose-response curves, indicative of efficient functional engagement of TCE002 with the corresponding pMHC comprising HLA-C*01:02 presenting the TP53 R273H-derived peptide. By contrast, other truncated TP53 R273H-derived peptides that retained the R273H substitution but differed in N-terminal and/or C-terminal flanking residues, including SEQ ID NO: 71 and SEQ ID NO: 73, induced minimal or no detectable cytotoxicity across the same TCE002 concentration range. Mock-treated controls likewise exhibited negligible background lysis. These results indicate that the presence of the TP53 R273H mutation alone is insufficient to confer functional recognition by TCE002. Rather, precise peptide length and flanking residue composition are critical determinants of productive pMHC formation and TCE002-mediated T cell activation and cytotoxicity.

### (J2) CD3 On-Cell Binding Assessment of Candidate TCE Constructs

Following the epitope mapping and HLA-restricted cytotoxicity analyses described above, the ability of the candidate TCE002 constructs to bind CD3 on the surface of immune effector cells was further evaluated to confirm effective T cell engagement independent of pMHC recognition.

Following the epitope mapping and HLA-restricted functional analyses described above, the CD3-binding properties of the candidate TCE constructs comprising TCE002A, TCE002B, TCE002C, and TCE002D were evaluated to confirm effective engagement of immune effector cells. CD3-positive cells were isolated and incubated with each candidate TCE construct at concentrations ranging from approximately 10⁻² nM to 102 nM for 30 minutes. After incubation, unbound constructs were removed by washing for three times with PBS 2% FBS, and bound TCE constructs were detected by staining cells with anti-Fc PE antibody at the ratio of 1:100 for 30 minutes. Binding was quantified by flow cytometry.

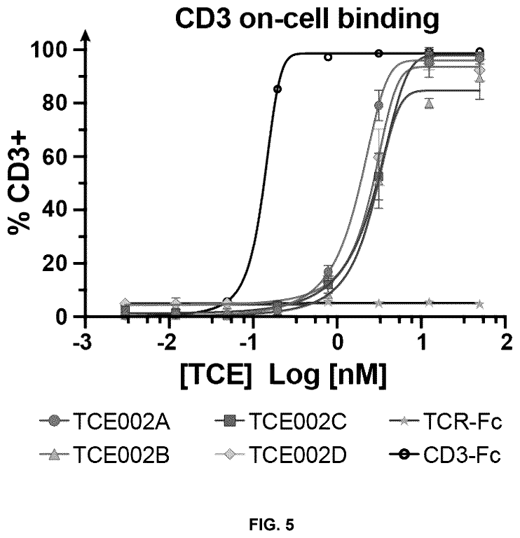

Referring to FIG. 5, each of the candidate TCE construct exhibited dose-dependent binding to CD3-positive cells, demonstrating sigmoidal binding curves characteristic of specific CD3 engagement. The observed binding profiles indicate that all four candidate TCE constructs retain functional anti-CD3 binding capability despite differences in TCR format (single-chain TCR versus stapled single-chain TCR) and domain arrangement. Comparative analysis showed that the CD3-binding efficiency of TCE002A and TCE002B was comparable to that of TCE002C and TCE002D, indicating that reduction in molecular size and alteration of domain order did not compromise CD3 engagement. A control TCR-Fc construct lacking an anti-CD3 binding domain displayed negligible binding across the tested concentration range, whereas a CD3-Fc positive control exhibited high-affinity binding, thereby confirming assay specificity. In some embodiments, the CD3-positive cells comprised primary human T cells isolated from PBMCs. In other embodiments, the CD3-positive cells comprised engineered cell lines expressing a CD3 complex, including HEK293T cells or SW620 colorectal carcinoma cells engineered to express CD3. Collectively, these results demonstrate that each of the candidate TCE constructs (TCE002A-TCE002D) maintains effective CD3 binding functionality. When considered together with the pMHC-specific binding and HLA-restricted cytotoxicity results described in FIG. 3 and FIGS. 4A-4B, the data confirm that TCE002 constructs enable coordinated dual engagement of target cells and immune effector cells.

It should be noted that TCR-Fc construct is used as controls and are encoded by nucleic acid sequences SEQ ID NO: 92 and corresponding polypeptide sequence SEQ ID NO: 93, respectively.

It should be noted that CD3-Fc construct is used as controls and are encoded by nucleic acid sequences SEQ ID NO: 94 and corresponding polypeptide sequence SEQ ID NO: 95, respectively.

(K2) Binding Assessment of Candidate TCE Constructs to TP53 R273H/HLA-C*01:02 pMHC

Following evaluation of CD3 on-cell binding, the binding properties of the candidate TCE constructs to the pMHC comprising HLA-C*01:02 presenting the TP53 R273H-derived 9-mer peptide were further assessed using both biophysical and cell-based assays.

### (K2.1) Biolayer Interferometry (BLI) Analysis

Binding affinity of the candidate TCE constructs to the TP53 R273H/HLA-C01:02 pMHC was evaluated by BLI. Briefly, GST-tagged HLA-C01:02 loaded with the TP53 R273H-derived 9-mer peptide was produced and immobilized on GST biosensors. His-tagged TCE002A, TCE002B, TCE002C, and TCE002D were prepared and tested as analytes. Loaded biosensors were immersed into wells containing three-fold serial dilutions of each TCE002 construct for an association phase of approximately 600 seconds, followed by a dissociation phase of approximately 600 seconds. Sensorgrams were recorded and analyzed to assess binding behavior.

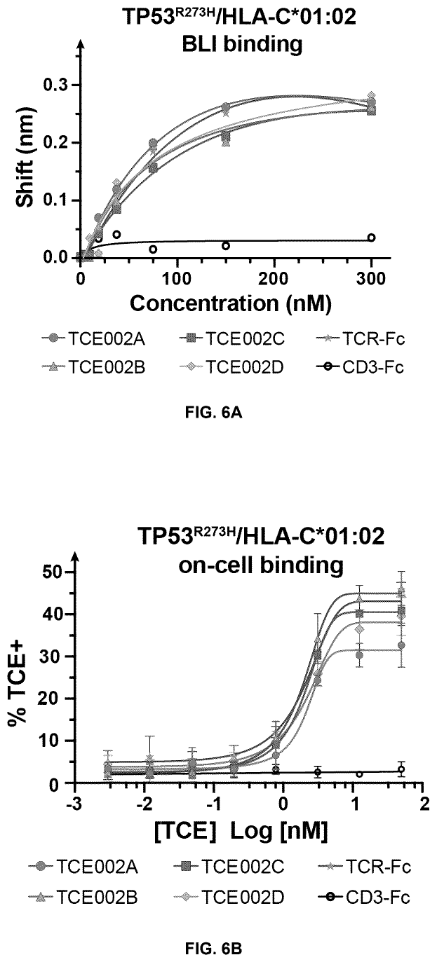

Referring to FIG. 6A, all candidate TCE constructs exhibited concentration-dependent binding to the TP53 R273H/HLA-C*01:02 pMHC, whereas control constructs lacking the TCR-derived binding domain showed negligible signal. These results demonstrate that incorporation of the optimized TCR sequence enables specific recognition of the pMHC complex at the biophysical level.

(K2.2) On-Cell pMHC Binding Analysis

To further evaluate pMHC recognition in a cellular context, binding of the TCE constructs to cells expressing HLA-C*01:02 presenting the TP53 R273H-derived 9-mer peptide was assessed by flow cytometry. Cells expressing HLA-C*01:02 were pulsed with the TP53 R273H-derived 9-mer peptide at a final concentration of approximately 20 μM in AIM-V™ medium supplemented with 1% FBS, and incubating pHLA-C*01:02/p53 R273H 9-mer cells with TCE construct in the range of 10⁻³-10² nM for 4 hours at room temperature with 5% CO₂. Peptide-pulsed cells were incubated with increasing concentrations of TCE002A, TCE002B, TCE002C, or TCE002D. To stabilize TCE-pMHC interactions, cells were cross-linked with formaldehyde at a final concentration of 1% (v/v) for 5 minutes in the dark. The cross-linking reaction was quenched by addition of Tris-HCl at pH 8.0 to a final concentration of 0.75 M, followed by incubation for 1 minute. Cells were then washed three times with PBS supplemented with 2% FBS, incubated in blocking buffer comprising PBS and 2% FBS for 30 minutes, and stained with an Fc-specific phycoerythrin (PE)-conjugated antibody at a dilution of 1:100 for 30 minutes. After additional washing steps, TCE binding to pMHC-expressing cells was quantified by flow cytometry.

As shown in FIG. 6B, all candidate TCE constructs demonstrated dose-dependent binding to cells presenting the TP53 R273H/HLA-C*01:02 PMHC, while minimal binding was observed for control constructs. The observed binding profiles confirm that the candidate TCE constructs retain pMHC recognition capability in a cell-based format. Collectively, the BLI-based binding data (FIG. 6A) and the on-cell pMHC binding results (FIG. 6B) demonstrate that the candidate TCE constructs specifically recognize the TP53 R273H-derived peptide presented by HLA-C*01:02.

### (I2) Assessment of T Cell Activation and TCE-Mediated Effector Function

Following confirmation of specific binding of the candidate TCE constructs to the TP53 R273H/HLA-C*01:02 peptide-major histocompatibility complex (pMHC), T cell activation and effector functionality were further evaluated using cell-based co-culture assays.

Antigen-presenting tumor cells pulsed with the TP53 R273H-derived peptide were co-cultured with primary human T cells in the presence of increasing concentrations of TCE002A, TCE002B, TCE002C, or TCE002D for approximately 24 hours, wherein the tumor cells are colorectal cancer cells SW620. Following co-culture, monensin was added to inhibit protein secretion from T cells for an additional incubation period of about 6 to 18 hours to allow intracellular accumulation of activation and effector molecules. T cells were then isolated from the co-culture, fixed with 4% formaldehyde for approximately 30 minutes, and washed three times with PBS supplemented with 2% FBS. The cells were incubated in a blocking buffer comprising PBS and 2% FBS for about 1 hour, followed by staining with fluorescently labeled antibodies specific for CD69, perforin, and granzyme B at a dilution of approximately 1:100 for about 30 minutes. After washing, expression of activation and cytotoxicity markers was quantified by flow cytometry to evaluate candidate TCE-mediated T cell activation and effector function.

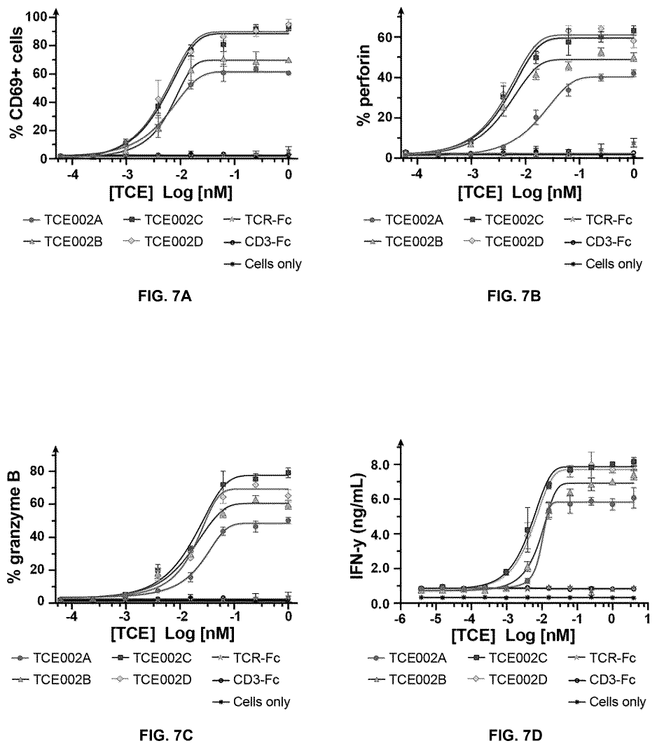

As shown in FIGS. 7A-7C, all candidate TCE constructs induced dose-dependent upregulation of CD69 expression and increased intracellular perforin and granzyme B levels relative to control conditions, indicating effective T cell activation upon engagement of the TP53 R273H/HLA-C*01:02 PMHC. Differences in activation potency and maximal response were observed among TCE002A-D, reflecting the influence of construct configuration on functional activity. In parallel, T cell effector function was assessed by measuring IFN-γ release from co-culture supernatants. As shown in FIG. 7D, TCE002-treated co-cultures exhibited robust, concentration-dependent IFN-γ secretion, whereas control treatments showed minimal cytokine release.

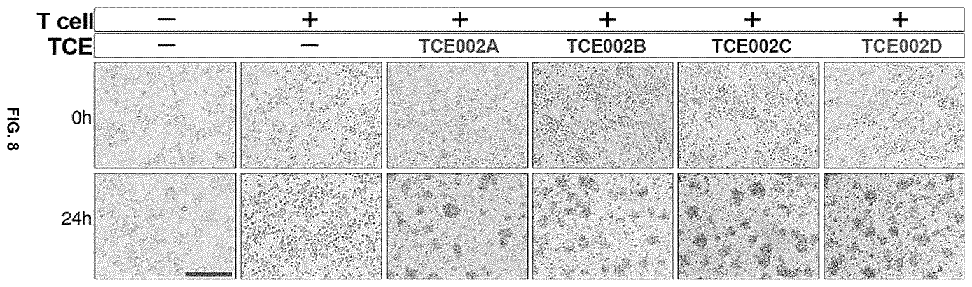

Consistent with these findings, microscopic analysis of tumor cell co-cultures revealed pronounced candidate TCE-dependent tumor cell killing after 24 hours, as illustrated in FIG. 8, while control conditions showed limited cytotoxic effects. These results demonstrate that the candidate TCE constructs not only bind the TP53 R273H/HLA-C*01:02 pMHC but also effectively trigger T cell activation and cytotoxic effector functions.

### (M2) Inter-Donor Variability of TCE-Mediated Cytotoxicity

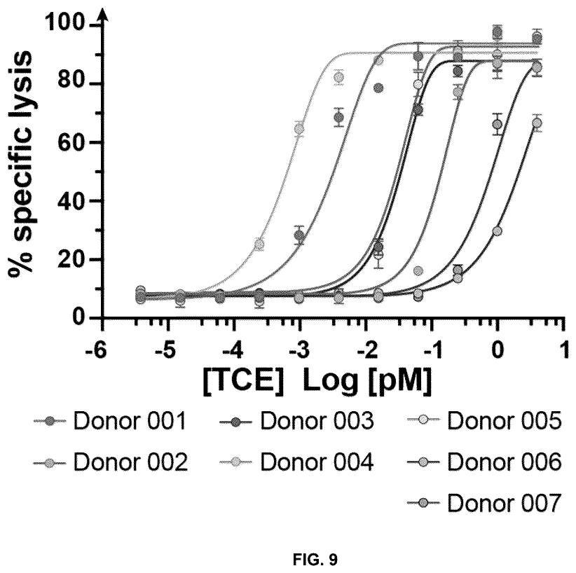

To further evaluate the robustness and donor-independence of TCE002C-mediated effector function, antigen-presenting tumor cells pulsed with the TP53 R273H-derived peptide were co-cultured with primary human T cells isolated from multiple independent donors in the presence of increasing concentrations of TCE002C. As shown in FIG. 9, TCE002C induced dose-dependent and specific tumor cell lysis across T cells derived from all tested donors. While the concentration-response profiles exhibited donor-to-donor variability in apparent potency, TCE002C consistently achieved high levels of maximal specific lysis at nanomolar to picomolar concentrations. These results demonstrate that TCE-mediated cytotoxic activity is reproducibly observed across genetically diverse T cell donors and is not restricted to a single donor source, thereby supporting the general applicability and functional robustness of TCE002C in mediating T cell-dependent tumor cell killing.

### (N2) Assessment of TCE Specificity Toward TP53 R273 9-Mer Variants

Following the evaluation of inter-donor variability, the specificity of TCE002C toward TP53 R273-derived peptide variants was further investigated. Tumor cells expressing HLA-C*01:02 were pulsed with a panel of TP53 R273 9-mer peptide variants, including the mutant R273H peptide (SEQ ID NO: 32) and alternative single-amino-acid substitutions at position 273 (SEQ ID NOs: 74-79) listed in Table 3 below, and subsequently evaluated for TCE002C-mediated functional activity and on-cell binding.

TABLE 3
TP53 R273 9-mer peptide variants
for evaluating TCE002C efficacy

| SEQ ID No. | Description | Symbol |
|---|---|---|
| 32 | TP53 R273H 9-mer peptide (H5) | R273H |
| 74 | Human TP53 R273 wildtype 9-mer peptide | R273WT |
| 75 | Human TP53 R273C 9-mer peptide | R273C |
| 76 | Human TP53 R273L 9-mer peptide | R273L |
| 77 | Human TP53 R273G 9-mer peptide | R273G |
| 78 | Human TP53 R273P 9-mer peptide | R273P |
| 79 | Human TP53 R273S 9-mer peptide | R273S |

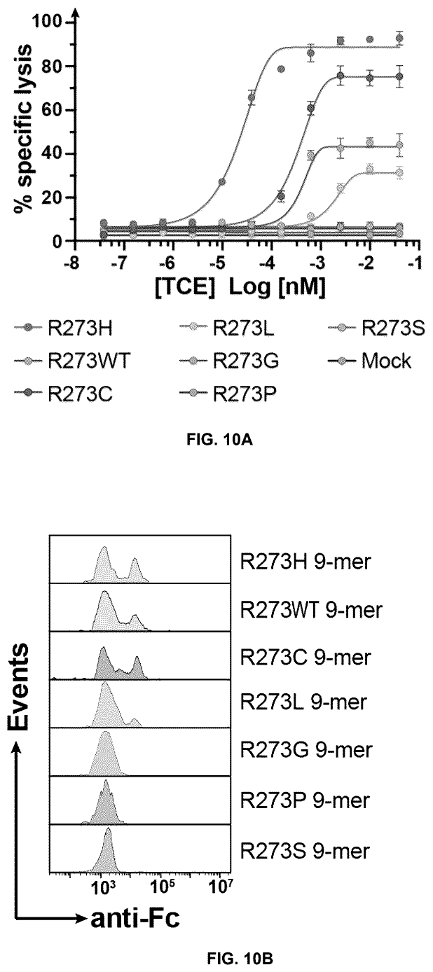

As shown in FIG. 10A, TCE002C induced potent and dose-dependent T cell mediated cytotoxicity exclusively against tumor cells presenting the TP53 R273H 9-mer peptide in complex with HLA-C*01:02. Specific lysis reached high levels at sub-nanomolar TCE002C concentrations, demonstrating strong functional engagement. In contrast, tumor cells pulsed with wild-type TP53 R273 peptides or alternative R273 variants (including R273L, R273S, R273G, R273P, and R273C) exhibited markedly reduced or negligible cytotoxicity across the same concentration range, comparable to mock-treated controls.

Consistent with the cytotoxicity data, FIG. 10B shows that TCE002C binding to pHLA-C*01:02 complexes was selectively observed on cells pulsed with the TP53 R273H 9-mer peptide, as detected by anti-Fc staining and flow cytometric analysis. Minimal or background-level binding was detected on cells presenting the wild-type or alternative R273 variant peptides. These results demonstrate that TCE002C exhibits high specificity for the TP53 R273H mutant epitope presented by HLA-C*01:02, with minimal cross-reactivity toward wild-type or closely related peptide variants.

### (P2) Off-Target Candidate Specificity Assessment

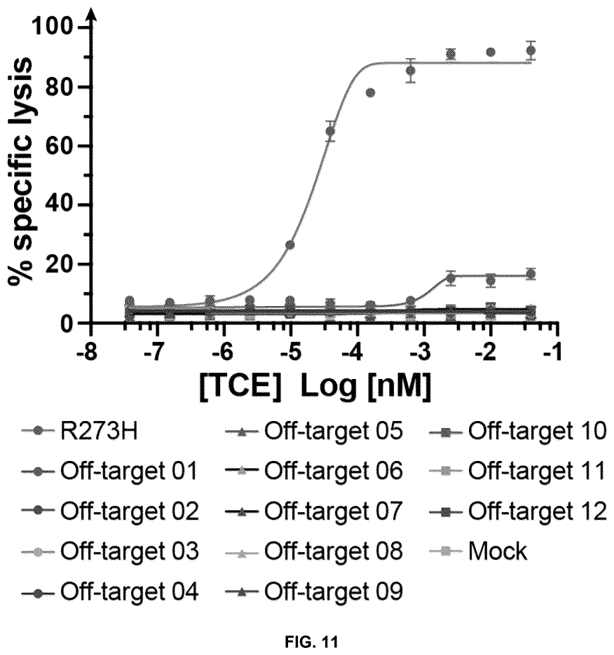

Referring to FIG. 11, TCE002C-mediated cytotoxicity was further evaluated using HLA-C*01:02-positive target cells pulsed with the TP53 R273H-derived 9-mer peptide (SEQ ID NO: 32) or with a panel of off-target candidate peptides (SEQ ID NOs: 80-91) listed in Table 4 below. TCE002C induced robust, dose-dependent specific lysis when target cells were pulsed with the R273H 9-mer peptide, reaching a high plateau at nanomolar-range concentrations. In contrast, target cells pulsed with the off-target candidate peptides generally exhibited minimal lysis comparable to mock, with only limited residual activity observed for certain candidates (e.g., off-target 01) at higher TCE002C concentrations. Collectively, these results demonstrate that TCE002C displays preferential functional recognition of the HLA-C*01:02/TP53 R273H 9-mer pMHC and reduced off-target reactivity, consistent with the specificity evaluation criterion.

TABLE 4
TP53 R273H off-target candidate peptides for evaluating TCE002C efficacy

| SEQ ID No. | Description | Symbol |
|---|---|---|
| 32 | TP53 R273H 9-mer peptide (H5) | R273H |
| 80 | [Off-target 01] Cadherin-13 | Off-target 01 |
| 81 | [Off-target 02] Tumor protein p73 | Off-target 02 |
| 82 | [Off-target 03] Integrin beta-6 | Off-target 03 |
| 83 | [Off-target 04] 1,4 alpha-glucan-branching enzyme | Off-target 04 |
| 84 | [Off-target 05] Tumor protein p63 C339S | Off-target 05 |
| 85 | [Off-target 06] Protein kinase C epsilon | Off-target 06 |
| 86 | [Off-target 07] Chromosome 18 putative open reading frame 15 | Off-target 07 |
| 87 | [Off-target 08] Tumor protein p63 wildtype | Off-target 08 |
| 88 | [Off-target 09] Ankyrin repeat and MYND domain-containing | Off-target 09 |
| | protein 1 | |
| 89 | [Off-target 10] PRAME family member 12 | Off-target 10 |
| 90 | [Off-target 11] Protein transport protein Sec16A | Off-target 11 |
| 91 | [Off-target 12] Fibronectin type III domain-containing protein 3B | Off-target 12 |

### (Q2) Alanine Scanning Analysis of the TP53 R273H 9-Mer Epitope

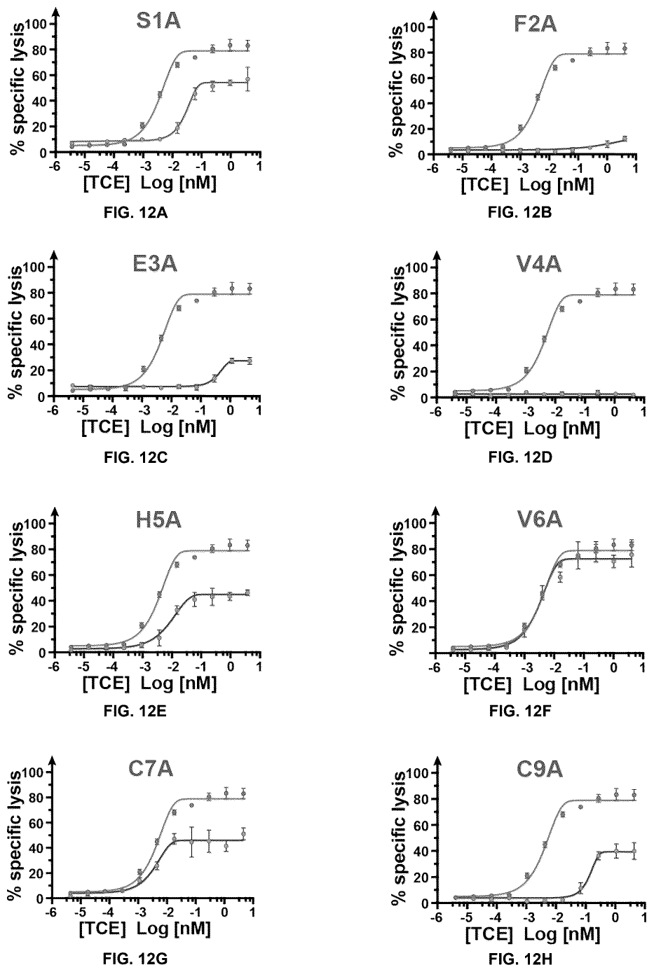

Referring to FIGS. 12A-12H, an alanine-scanning analysis was performed across the TP53 R273H-derived 9-mer peptide H5 (SEQ ID NO: 32) to identify amino acid residues critical for TCE002C-mediated T cell cytotoxicity in the context of HLA-C01:02 presentation. Each residue of the 9-mer peptide was individually substituted with alanine (SEQ ID NOs: 34-41) listed in Table 5 below, and HLA-C01:02-positive target cells pulsed with the corresponding alanine-substituted peptides were evaluated in TCE002C-mediated cytotoxicity assays. The results demonstrate that substitution of specific residues within the 9-mer peptide resulted in a marked reduction or complete abrogation of T cell-dependent killing, indicating that these positions are essential for functional recognition by TCE002. In contrast, alanine substitution at other positions partially preserved cytotoxic activity, suggesting a contributory but non-essential role in TCE002 engagement.

TABLE 5
Peptide used for alanine scan of p53 R273H 9-mer peptide H5

| SEQ ID No. | Description | Symbol |
|---|---|---|
| 32 | TP53 R273H 9-mer peptide (H5) | — |
| 34 | Alanine scan for TP53 R273H 9-mer (H5) peptide, position S1 | S1A |
| 35 | Alanine scan for TP53 R273H 9-mer (H5) peptide, position F2 | F2A |
| 36 | Alanine scan for TP53 R273H 9-mer (H5) peptide, position E3 | E3A |
| 37 | Alanine scan for TP53 R273H 9-mer (H5) peptide, position V4 | V4A |
| 38 | Alanine scan for TP53 R273H 9-mer (H5) peptide, position H5 | H5A |
| 39 | Alanine scan for TP53 R273H 9-mer (H5) peptide, position V6 | V6A |
| 40 | Alanine scan for TP53 R273H 9-mer (H5) peptide, position C7 | C7A |
| 41 | Alanine scan for TP53 R273H 9-mer (H5) peptide, position C9 | C9A |

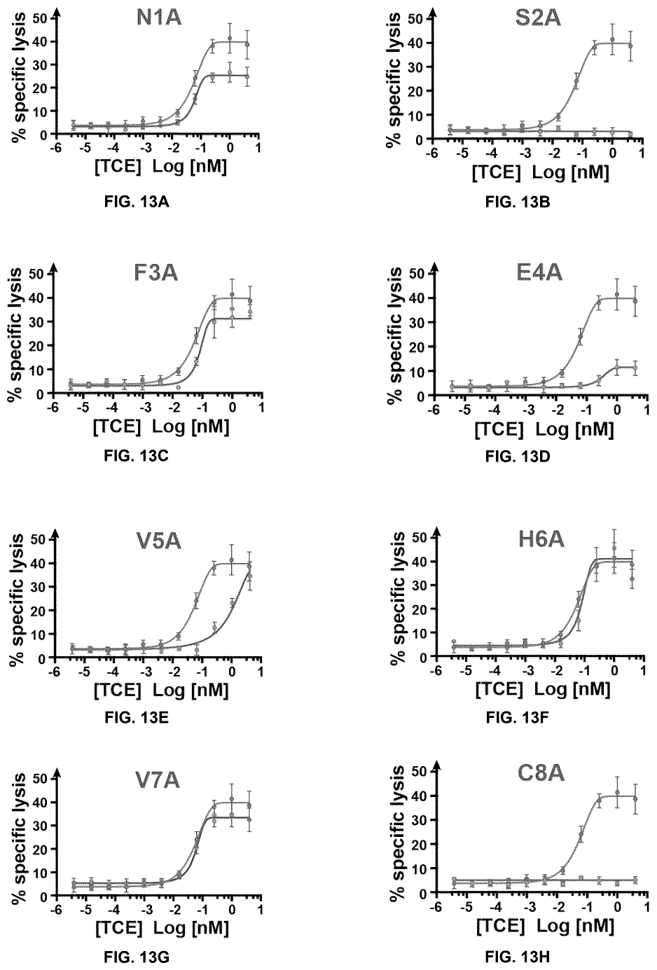

Following the alanine scanning analysis of the TP53 R273H-derived 9-mer epitope (H5) shown in FIG. 12, a second TP53 R273H-derived 9-mer peptide (H6) (SEQ ID NO: 33), was subjected to systematic alanine substitution to further define residue-level determinants of TCE002C functional activity. As illustrated in FIGS. 13A-13H, each amino acid position within the H6 epitope was individually substituted with alanine (SEQ ID NOs: 42-49) listed in Table 6, and the resulting peptides were evaluated for TCE002C-mediated T cell-dependent cytotoxicity against target cells expressing pHLA-C*01:02. The results demonstrate that alanine substitutions at different positions within the H6 epitope produced heterogeneous effects on TCE002C activity. Certain substitutions preserved dose-dependent cytotoxic responses with measurable maximal lysis, whereas substitutions at other positions markedly reduced or abolished TCE002C-mediated killing across the tested concentration range. These findings indicate that specific residues within the H6 epitope are critical for productive TCE002C engagement, while other positions are more tolerant to substitution.

TABLE 6
Peptide used for alanine scan of p53 R273H 9-mer peptide H6

| SEQ ID No. | Description | Symbol |
|---|---|---|
| 33 | TP53 R273H 9-mer peptide (H6) | — |
| 42 | Alanine scan for TP53 R273H 9-mer (H6) peptide, position N1 | N1A |
| 43 | Alanine scan for TP53 R273H 9-mer (H6) peptide, position S2 | S2A |
| 44 | Alanine scan for TP53 R273H 9-mer (H6) peptide, position F3 | F3A |
| 45 | Alanine scan for TP53 R273H 9-mer (H6) peptide, position E4 | E4A |
| 46 | Alanine scan for TP53 R273H 9-mer (H6) peptide, position V5 | V5A |
| 47 | Alanine scan for TP53 R273H 9-mer (H6) peptide, position H6 | H6A |
| 48 | Alanine scan for TP53 R273H 9-mer (H6) peptide, position V7 | V7A |
| 49 | Alanine scan for TP53 R273H 9-mer (H6) peptide, position C8 | C8A |

(R2) in-Silico Optimization and Expansion of the TCE002 Variant Families (Also Known as TCE Variant Families)

Following functional characterization of the initial TCE002 constructs (also known as candidate TCE constructs), including TCE002A, TCE002B, TCE002C, and TCE002D, further optimization was conducted to improve binding performance, functional activity, and developability characteristics while preserving specificity toward the pMHC comprising HLA-C*01:02 presenting the TP53 R273H-derived 9-mer peptide.

(R2.1) in-Silico Optimization Strategy

In certain embodiments, optimization of the TCE002 constructs was performed using an in-silico directed evolution workflow, comprising the following steps:
(f1) predicting a three-dimensional (3D) structure of HLA-C*01:02 (SEQ ID NO: 97) presenting the TP53 R273H-derived 9-mer peptide H5 (SEQ ID NO: 32);
(f2) predicting a 3D structural complex of a TCE002 construct bound to a pHLA-C*01:02/TP53 R273H tetramer;
(f3) performing in-silico directed evolution of both α- and β-chain variable regions of the parental TCR sequence (TCR002), wherein up to approximately 50 amino acid substitutions per chain were introduced, and wherein mutations were distributed across the variable regions and were not limited to complementarity-determining regions (CDRs);
(f4) predicting binding affinity of the newly generated TCR variants toward the pHLA-C*01:02/TP53 R273H complex under multiple simulated interaction conditions;
(f5) predicting immunogenicity scores of the newly generated TCR variants;
(f6) predicting humanness scores of the newly generated TCR variants;
(f7) predicting aggregation propensity scores of the newly generated TCR variants; and
(f8) ranking the newly generated TCR variants based on a composite assessment of predicted binding affinity, immunogenicity, humanness, and aggregation propensity.
   (R2.2) Iterative in-Silico Directed Evolution Workflow

In certain embodiments, the optimization process comprised engineering a TCE construct incorporating a top-ranked TCR variant, followed by experimental verification of its binding and functional efficacy. The experimentally validated top-performing TCR variant was subsequently used as the foundation for a next round of in-silico directed evolution, by repeating sub-steps (f1)-(f8) described above. This iterative process was continued until no additional higher-ranked TCR variants were suggested by the in-silico workflow.

In some embodiments, the in-silico directed evolution process was performed for three successive rounds, resulting in the identification of multiple generations of optimized TCR variants (also known as candidate optimized TCR) selected from SEQ ID NOs: 5-31, including TCR002.1-TCR002.13, TCR002.5.1-TCR002.5.10, and TCR002.5.4.1.

The optimized TCR variants generated across these rounds were incorporated into TCE constructs, yielding a family of optimized TCEs encoded by nucleic acid and polypeptide sequences selected from SEQ ID NOs: 56-61, and 50-55.

### (R2.3) Biophysical and Cellular Binding Characterization of Optimized TCEs

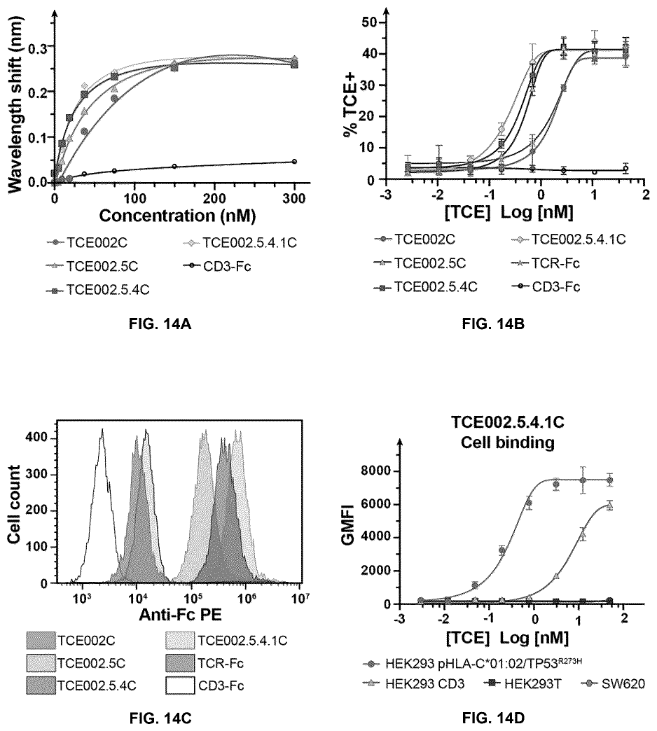

Binding of the optimized TCE constructs toward the pHLA-C*01:02/TP53 R273H 9-mer complex was evaluated using BLI, as illustrated in FIG. 14A. The binding responses of TCE002C and the successive optimized variants (TCE002.5C, TCE002.5.4C, and TCE002.5.4.1C) exhibited concentration-dependent profiles with saturation concentration, consistent with specific engagement of the target pMHC. Relative to the earlier-generation construct, selected optimized variants demonstrated enhanced binding characteristics, whereas control constructs (e.g., CD3-Fc) exhibited baseline or minimal signal under the same conditions.

Cell-based binding efficiency of the optimized TCEs was further assessed using cells expressing pHLA-C*01:02 presenting the TP53 R273H 9-mer peptide (H5). In-cell binding was quantified by flow cytometry, as shown in FIG. 14B and FIG. 14C. In addition, relative binding selectivity of the optimized TCEs toward pHLA-positive cells versus CD3-positive cells was evaluated, as illustrated in FIG. 14D, confirming preferential engagement of the target pMHC.

### (R2.4) Functional Evaluation of Optimized TCE Variants

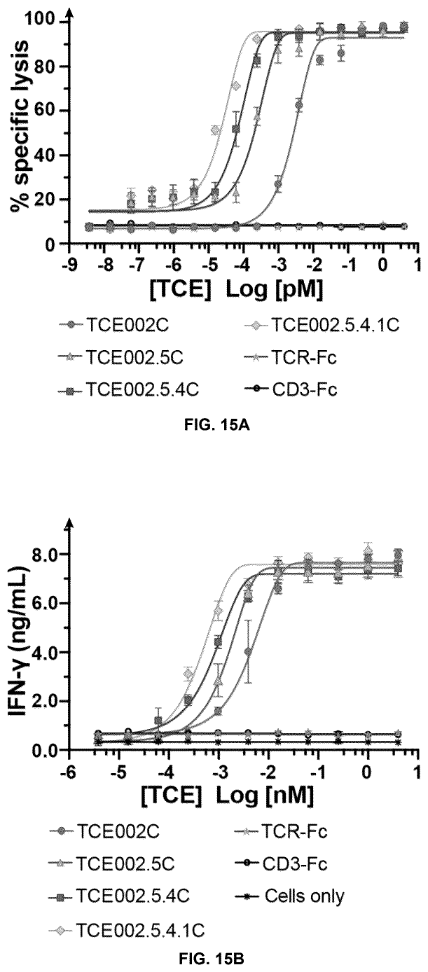

Functional activity of representative TCE constructs derived from TCE002C was evaluated in TCE-mediated co-culture assays consistent with the evaluation criteria described herein. Tumor cell killing was quantified using a luminescent cell viability assay to determine percent specific lysis, and T cell activation was assessed by measurement of IFN-γ secretion. As shown in FIG. 15A, TCE002C and successive TCE constructs incorporating in-silico-generated TCR variants (TCE002.5C, TCE002.5.4C, and TCE002.5.4.1C) mediated dose-dependent tumor cell lysis and reached high maximal lysis under the tested conditions, with later-generation constructs achieving comparable lysis at lower TCE concentrations. As shown in FIG. 15B, the same series of TCE constructs induced concentration-dependent IFN-γ secretion in co-culture, whereas control constructs (TCR-Fc and CD3-Fc) and cells-only controls exhibited baseline responses.

### (R2.5) Evaluation of Optimized TCE Specificity Toward TP53 R273H 9-Mer Variants

To further assess epitope specificity following multi-round in-silico optimization, an optimized TCE construct incorporating the TCR002.5.4.1 variant (TCE002.5.4.1C) was evaluated for functional activity and on-cell binding using HLA-C01:02-positive target cells pulsed with TP53 R273H-derived 9-mer peptide variants (H5) (SEQ ID NOs: 32 and 74-79).

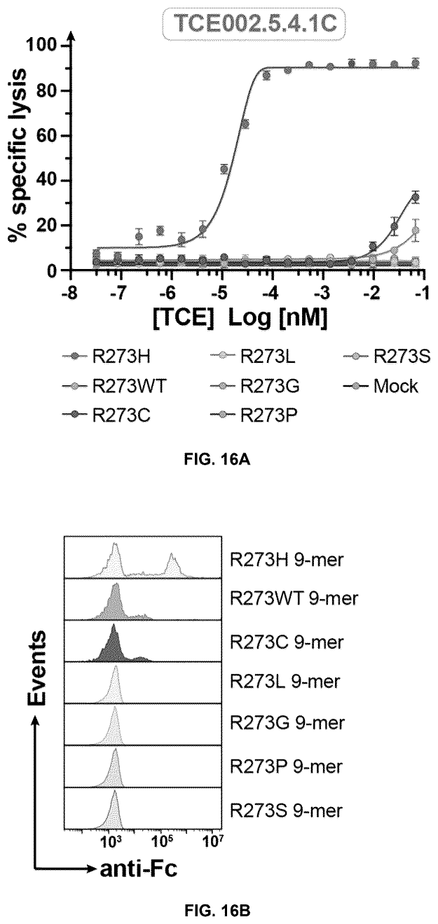

As shown in FIG. 16A, target cells pulsed with the R273H-containing 9-mer peptide exhibited a clear dose-dependent increase in T cell-mediated cytotoxicity and reached high maximal specific lysis under the tested conditions, whereas target cells pulsed with alternative R273-substituted 9-mer variants and mock-treated controls exhibited substantially reduced or baseline lysis. Consistent with these functional results, FIG. 16B shows that TCE002.5.4.1C produced the strongest on-cell binding signal on cells presenting the R273H-containing 9-mer peptide, with markedly lower binding signals observed for the other R273-substituted variants.

(R2.6) Evaluation of Characterize Specificity of the TCE Construct with Respect to Off-Target Peptide Recognition, HLA Restriction, and Peptide Dose Dependency

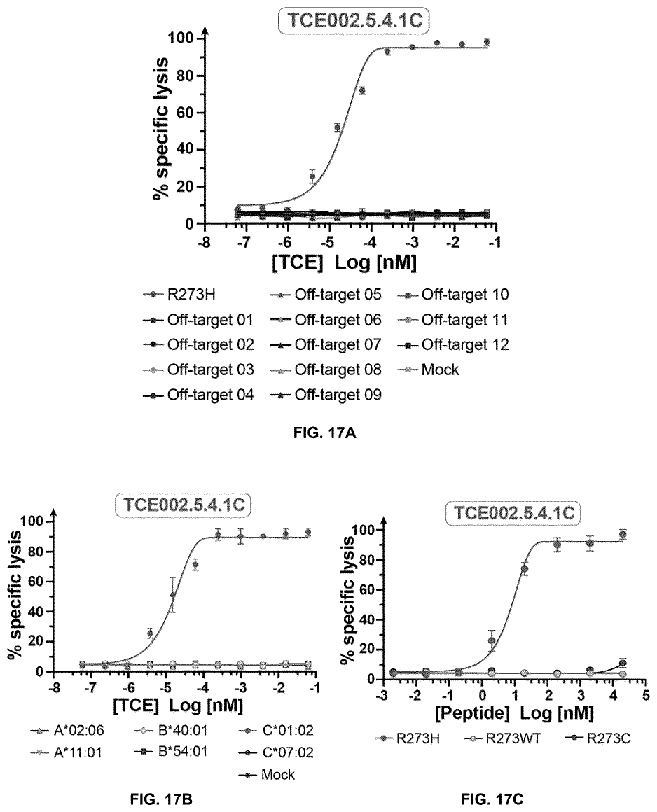

Specificity of TCE002.5.4.1C was evaluated across (i) an off-target peptide panel, (ii) a panel of cells expressing distinct HLA class I alleles, and (iii) varying peptide pulsing doses, as shown in FIGS. 17A-17C. Referring to FIG. 17A, TCE002.5.4.1C mediated dose-dependent T cell-dependent cytotoxicity against target cells presenting the TP53 R273H-derived 9-mer peptide, whereas cytotoxicity remained at low background levels for the tested off-target candidate peptides (SEQ ID NOs: 80-91) and mock controls. Referring to FIG. 17B, cytotoxicity was observed predominantly on target cells presenting a pMHC comprising HLA-C01:02 (SEQ ID NO: 97), while target cells expressing other tested HLA alleles (SEQ ID NOs: 96-119) exhibited negligible cytotoxicity under the same conditions, consistent with HLA-restricted recognition. Referring to FIG. 17C, TCE002.5.4.1C activity on HLA-C01:02-positive target cells increased with increasing pulsed peptide dose for the TP53 R273H-derived 9-mer peptide (SEQ ID NO: 32), while responses to the TP53 R273WT 9-mer peptide (SEQ ID NO: 74) and the TP53 R273C 9-mer peptide (SEQ ID NO: 75) remained substantially lower across the tested peptide concentration range.

### (R2.7) Alanine Scanning Analysis of the TP53 R273H H5 Epitope

To further define residue-level determinants that support TCE002.5.4.1C activity in the context of a pMHC comprising HLA-C*01:02 presenting the TP53 R273H-derived 9-mer (H5) peptide (SEQ ID NO: 32), an alanine-scanning series was generated by individually substituting each residue of the (H5) peptide with alanine (SEQ ID NOS: 34-41). Target cells expressing HLA-C*01:02 were pulsed with the alanine-substituted peptides and evaluated in TCE002.5.4.1C-mediated co-culture cytotoxicity assays, with percent specific lysis measured across a concentration range of TCE002.5.4.1C.

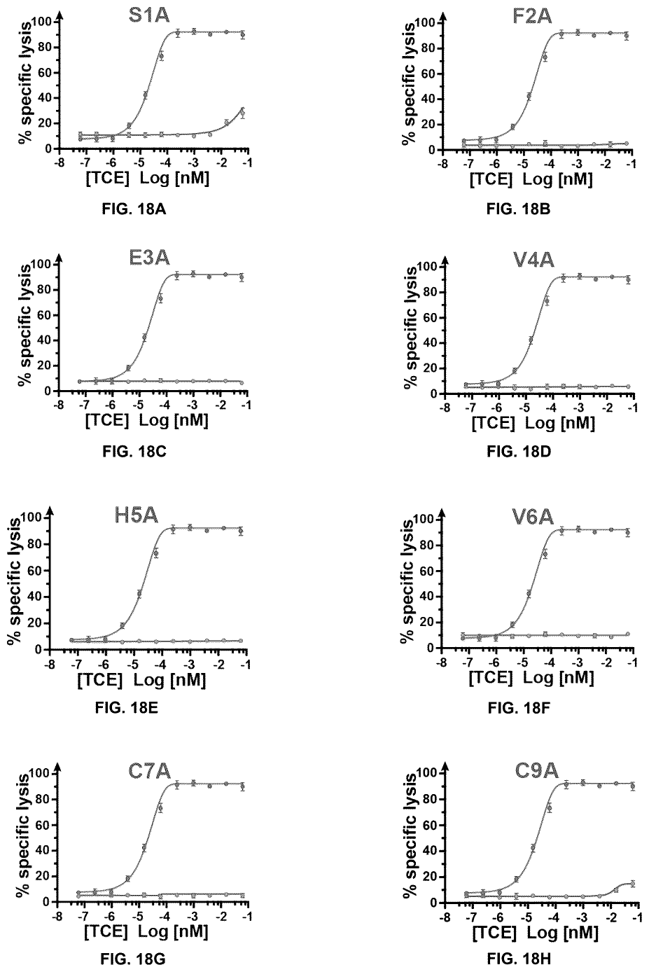

Referring to FIGS. 18A-18H, the parental (H5) peptide supported robust, concentration-dependent cytotoxicity, whereas alanine substitution at multiple positions markedly reduced or abrogated TCE002.5.4.1C-mediated killing, indicating that TCE002.5.4.1C functional activity is sensitive to defined residues within the H5 sequence. Certain alanine substitutions retained only residual activity at higher TCE concentrations, consistent with reduced potency and/or reduced maximal response relative to the parental peptide. Collectively, these results demonstrate that TCE002.5.4.1C activity depends on the precise TP53 R273H (H5) peptide sequence presented by HLA-C*01:02, supporting epitope-specific recognition in a peptide-HLA context. Results demonstrate that iterative in-silico directed evolution, coupled with experimental validation at each round, enables systematic optimization of TCE constructs across multiple generations. The resulting TCE002.5, TCE002.5.4, and TCE002.5.4.1 variants represent a coherent family of optimized TCE constructs, maintaining defined construct architectures while exhibiting enhanced binding affinity, functional activity, and specificity toward the pHLA-C*01:02/TP53 R273H epitope.

### (R2.8) Evaluation Across Malignant and Non-Malignant Cell Types

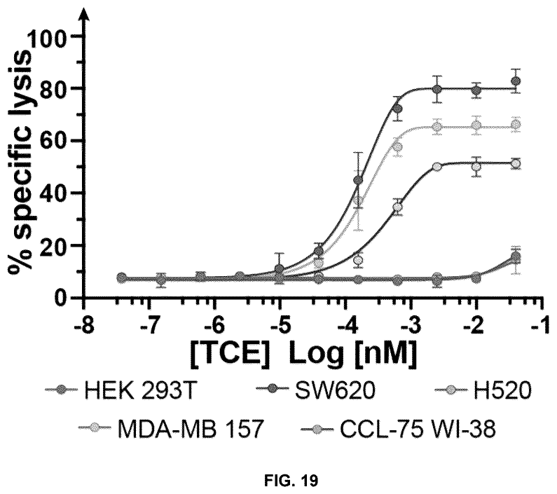

The activity of TCE002.5.4.1C was further evaluated across multiple cell types in TCE-mediated co-culture cytotoxicity assays, including SW620 colorectal carcinoma cells, H520 lung squamous carcinoma cells, and MDA-MB 157 breast cancer cells, as well as non-cancerous control cell lines including HEK293T cells and CCL-75 WI-38 lung fibroblasts. As illustrated in FIG. 19, TCE002.5.4.1C mediated dose-dependent cytotoxicity on the tumor cell lines, with the highest specific lysis observed for SW620 cells, whereas non-cancerous control cells exhibited only baseline lysis across the tested concentration range, supporting functional selectivity of TCE002.5.4.1C under the assay conditions.

### (R2.9) Assessment of Endogenous TP53 R273 Mutational Status in Distinct HLA.

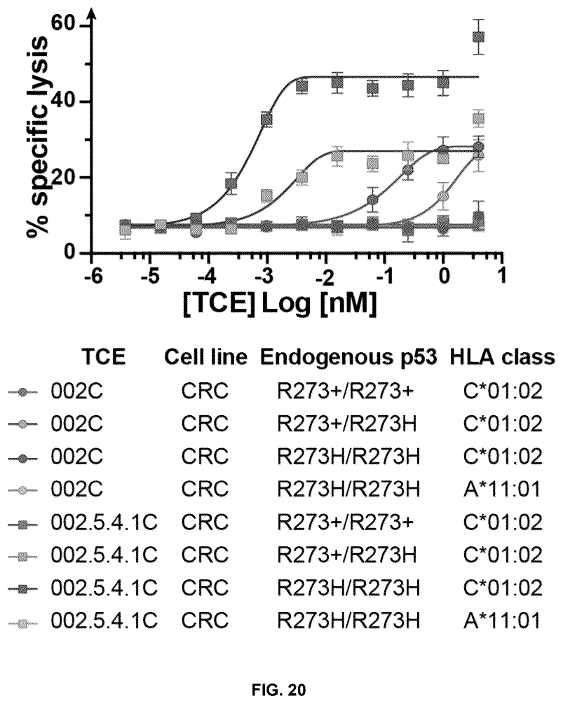

As shown in FIG. 20, the TCE-mediated T cell cytotoxic activity of TCE002C and the optimized TCE002.5.4.1C construct against tumor cells expressing endogenous TP53 variants under different HLA class I contexts. Specifically, SW620 colorectal cancer cell lines carrying distinct endogenous TP53 genotypes, including TP53 wild-type, heterozygous TP53 R273H, or compound heterozygous TP53 R273H, were evaluated in the presence of either HLA-C01:02 or HLA-A11:01. The TCE002.5.4.1C induced robust and concentration-dependent T cell-mediated cytotoxicity toward tumor cells expressing HLA-C01:02 in combination with endogenous TP53 R273H alleles, whereas minimal cytotoxic activity was observed against cells expressing HLA-A11:01 or lacking the TP53 R273H mutation. In contrast, TCE002C exhibited comparatively reduced killing efficiency under the same experimental conditions. These results demonstrate that the optimized TCE002.5.4.1C construct retains functional activity in the context of endogenously expressed TP53 R273H alleles and exhibits strict HLA-C*01:02 restriction, thereby confirming that TCE activity is dependent on both the presence of the TP53 R273H epitope and the appropriate presenting HLA allele.

### (R2.10) Assessment Functional Stability of Optimized TCE Constructs

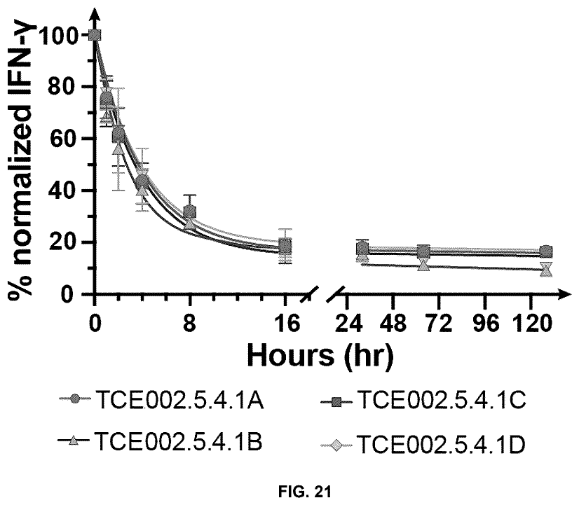

Each TCE construct was incubated at a concentration of 1.0 nM in human serum at 37 ° C for a duration ranging from 0 to 128 hours. At designated time points, residual functional activity of the serum-incubated TCEs was assessed using T cell co-culture assays performed in a manner consistent with the cytotoxicity and cytokine release. Functional stability was quantified by measuring T cell-derived IFN-γ release following TCE-mediated engagement of antigen-presenting target cells. IFN-γ levels obtained at each incubation time point were normalized relative to the signal measured at time zero (t=0 h), thereby providing a comparative assessment of functional activity retention over time. As shown in FIG. 21, TCE002.5.4.1A-D constructs exhibited a gradual, time-dependent reduction in functional activity during serum incubation, while maintaining measurable IFN-γ induction even after extended exposure to human serum.

### (S2) 3D Spheroid Cytotoxicity Assessment

To evaluate TCE activity in a three-dimensional tumor microenvironment model, multicellular spheroids were generated comprising (i) adipose-derived stem cells (ASC), (ii) human umbilical vein endothelial cells (HUVECs), and (iii) SW620 colorectal carcinoma cells presenting a pMHC comprising HLA-C*01:02 loaded with a TP53 R273H-derived 9-mer peptide (H5). Spheroids were co-cultured with primary human T cells in the presence of increasing concentrations of TCE002C, and functional outcomes were assessed by imaging, a three-dimensional viability assay, and IFN-γ secretion.

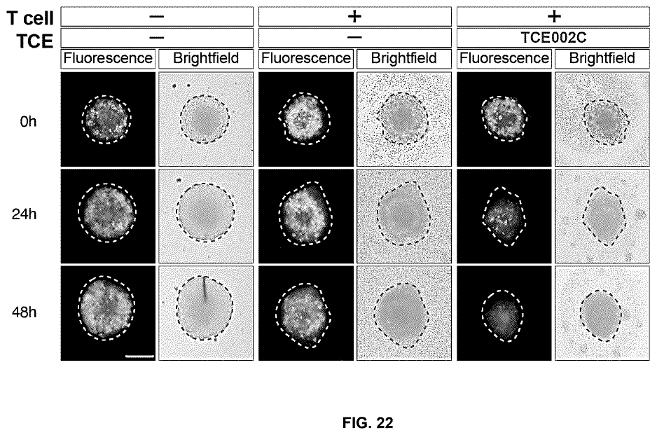

Referring to FIG. 22, representative fluorescence and brightfield images of the spheroids acquired at 0 h, 24 h, and 48 h show that, in the presence of T cells plus TCE002C, spheroids exhibited visible structural disruption over time relative to control conditions lacking TCE and/or T cells. The time-dependent morphological changes are consistent with TCE002C-mediated immune cell engagement and subsequent target-cell damage within the spheroid context.

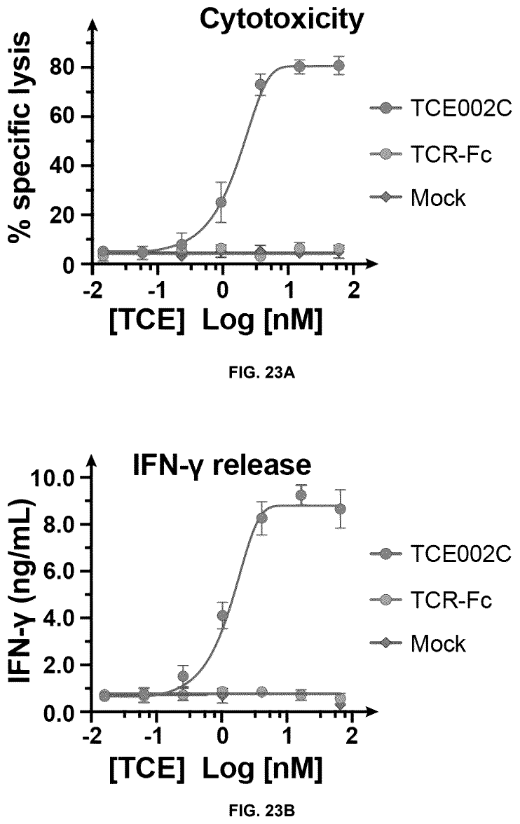

Referring to FIG. 23A, TCE002C induced dose-dependent T cell-mediated cytotoxicity against the 3D spheroids, as quantified by a luminescent 3D cell viability assay normalized to a mock condition. In contrast, control constructs (e.g., TCR-Fc) and mock conditions did not elicit appreciable killing across the tested concentration range, supporting that the observed effect is dependent on the bispecific engager function of TCE002C in the co-culture setting.

Referring to FIG. 23B, TCE002C also produced a concentration-dependent increase in IFN-γ secretion in spheroid co-cultures, consistent with TCE-driven T cell activation in the 3D model. Control conditions remained near baseline, supporting that cytokine release correlates with TCE002C-mediated engagement and functional activity.

Results demonstrate that TCE002C retains functional activity in a multicellular 3D spheroid format and mediates both (i) measurable target-cell killing and (ii) T cell activation, thereby supporting TCE performance under conditions that more closely model complex tumor architecture than two-dimensional assays.

### (T2) Evaluation of TCE002.5.4.1C Activity in 3D Tumor Spheroid Models and Combination Therapy

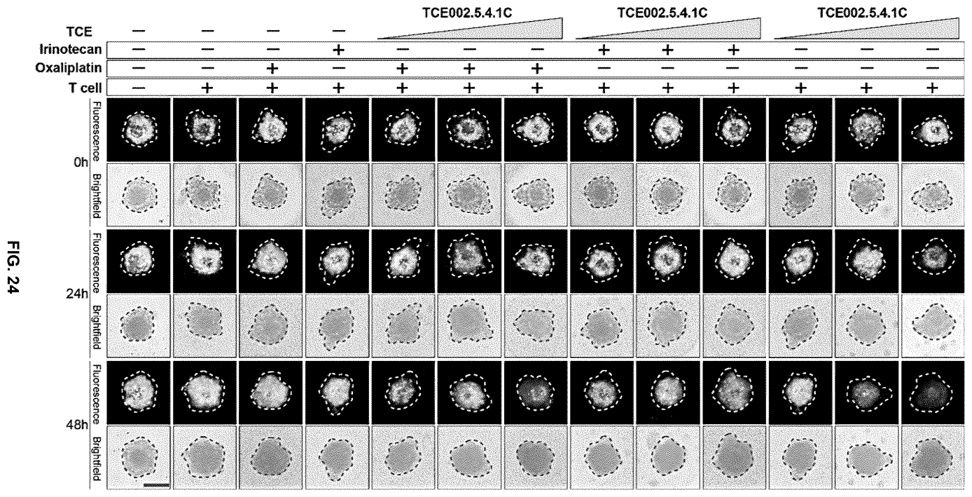

Referring to FIG. 24, the functional efficacy of the optimized TCE construct TCE002.5.4.1C was evaluated in 3D tumor spheroid model designed to recapitulate key features of the tumor microenvironment. Each spheroid comprised adipose-derived stem cells (ASCs), human umbilical vein endothelial cells (HUVECs), and SW620 colorectal cancer cells expressing HLA-C*01:02 presenting the TP53 R273H 9-mer peptide (H5). Spheroids were co-cultured with primary human T cells and treated with TCE002.5.4.1C either alone or in combination with chemotherapeutic agents, including irinotecan or oxaliplatin, over a treatment period of up to 48 hours. Fluorescence and brightfield imaging at 0 hours, 24 hours, and 48 hours demonstrated progressive spheroid disruption and loss of structural integrity in the presence of TCE002.5.4.1C, consistent with T cell-mediated cytotoxic activity. Notably, spheroids treated with TCE002.5.4.1C in combination with either irinotecan or oxaliplatin exhibited more pronounced reductions in spheroid size and cellular cohesion compared to treatment with TCE002.5.4.1C alone, indicating an enhanced cytotoxic effect under combination treatment conditions.

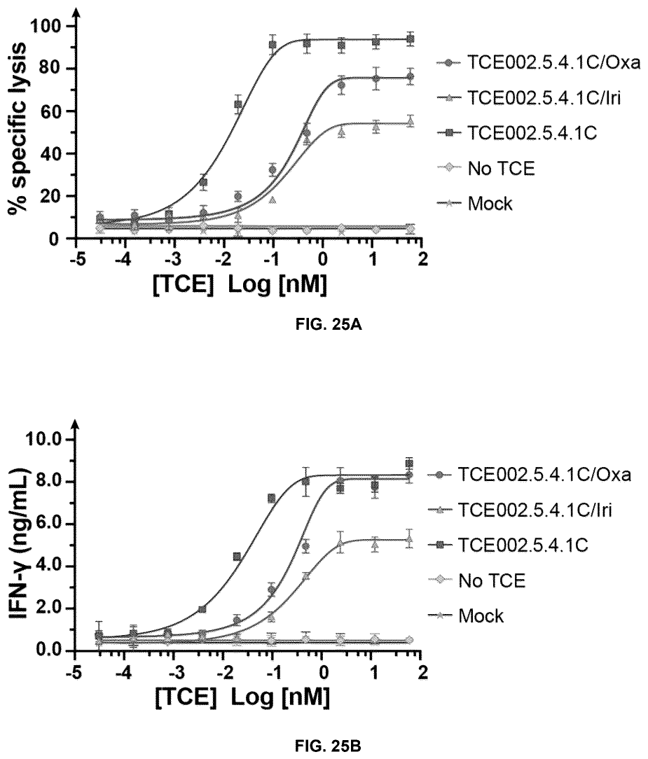

Referring to FIG. 25A, quantitative analysis of spheroid viability at 48 hours using a luminescent cell viability assay demonstrated dose-dependent T cell-mediated killing in response to TCE002.5.4.1C. TCE002.5.4.1C alone induced substantial tumor cell killing relative to mock-treated controls, while combination treatment with irinotecan or oxaliplatin further increased cytotoxic efficacy across the tested TCE concentration range. These results indicate additive or synergistic effects between TCE-mediated immune engagement and conventional chemotherapeutic agents.

Referring to FIG. 25B, T cell activation in the 3D spheroid co-culture system was further assessed by measuring IFN-γ secretion in the culture supernatant at 48 hours. TCE002.5.4.1C induced robust, concentration-dependent IFN-γ release, consistent with effective T cell activation upon engagement of the target pHLA complex. Combination treatment with irinotecan or oxaliplatin resulted in increased IFN-γ secretion compared to TCE002.5.4.1C alone, further supporting enhanced immune activation in the combination setting.

### (U2) Evaluation of TCE002.5.4.1C Activity on 3D Spheroids Harboring Endogenous TP53 R273H Mutations

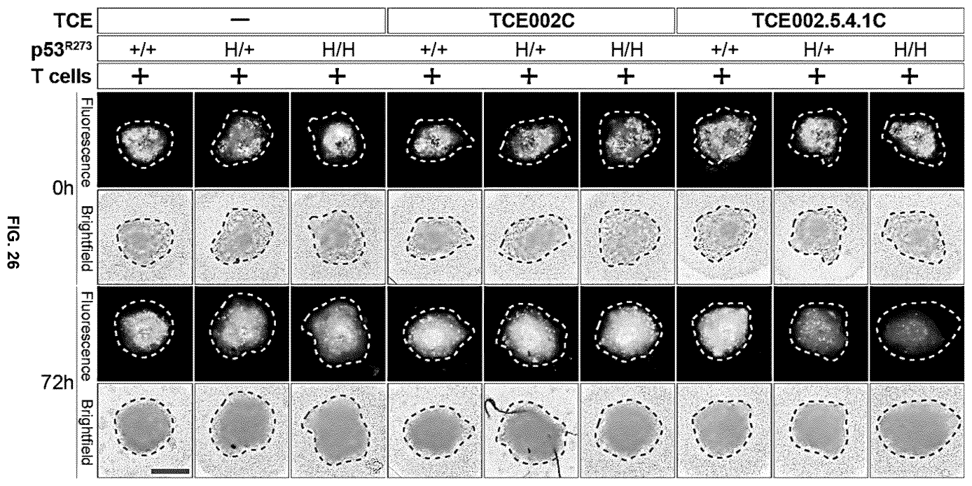

Referring to FIG. 26, the efficacy of TCE constructs was further evaluated in 3D tumor spheroid model comprising tumor cells endogenously expressing TP53 R273 variants, together with supporting stromal components and primary human T cells. The spheroids were treated with TCE002.5.4.1C or the parental construct TCE002C, in the presence or absence of T cells, and monitored over time by fluorescence and brightfield microscopy at 0 hours and 72 hours. Results spheroids expressing endogenous TP53 R273H mutations exhibited pronounced structural disruption and loss of integrity following treatment with TCE002.5.4.1C in the presence of T cells, whereas spheroids treated with TCE002C or lacking T cells retained compact morphology and showed minimal changes. These observations demonstrate that TCE002.5.4.1C mediates effective T cell-dependent cytotoxic activity within a 3D tumor microenvironment, including against tumor cells harboring endogenously expressed mutant TP53.

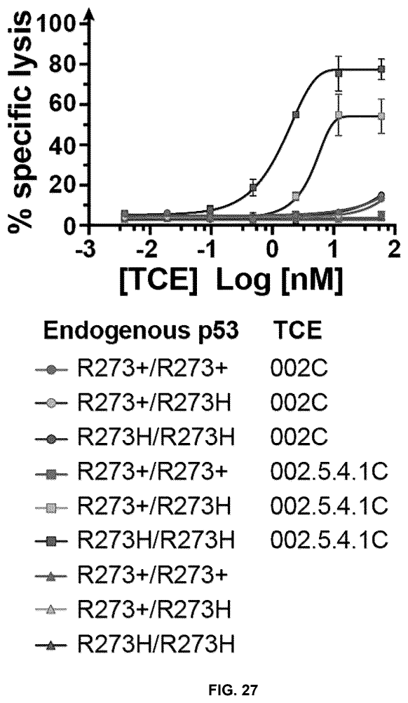

Referring to FIG. 27, the functional efficacy of TCE002.5.4.1C was quantitatively assessed by measuring T cell-mediated cytotoxicity against tumor cells with different endogenous TP53 genotypes, including TP53 wild-type (R273+/R273+), heterozygous TP53 R273H (R273+/R273H), and homozygous TP53 R273H (R273H/R273H), in the context of HLA-C*01:02 expression. TCE002.5.4.1C induced robust, dose-dependent tumor cell killing selectively in spheroids harboring heterozygous or homozygous TP53 R273H mutations, with markedly enhanced efficacy relative to the parental TCE002C construct. In contrast, tumor cells expressing wild-type TP53 showed minimal cytotoxicity across the tested concentration range.

The results shown in FIG. 26 and FIG. 27 demonstrate that TCE002.5.4.1C retains high functional potency and selectivity in complex 3D tumor models and effectively discriminates tumor cells based on endogenous TP53 R273H mutation status, thereby supporting its suitability for targeting solid tumors expressing naturally occurring TP53 R273H mutations in an HLA-C*01:02-restricted manner.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, element components, and/or groups thereof.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application and to enable others of ordinary skill in the art to understand the invention for various embodiments with various modifications as are suited to the particular use contemplated.

The flow diagrams depicted herein are just one example. There may be many variations to this diagram or the steps (or operations) described therein without departing from the spirit of the invention. For instance, the steps may be performed in a differing order, or steps may be added, deleted, or modified. All of these variations are considered a part of the claimed invention.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A method for identifying and optimizing an engineered T cell engager receptor (TCE) specifically recognizing a peptide-major histocompatibility complex (pMHC) comprising HLA-C*01:02 presenting a TP53 R273H-derived 9-mer peptide, comprising:
(a) generating a plurality of candidate optimized T cell receptor (TCR) sequences by modifying a TCR sequence capable of recognizing the pMHC comprising HLA-C*01:02 presenting the TP53 R273H-derived 9-mer peptide;
wherein the TCR sequence is selected from the group consisting of SEQ ID NOs: 1-4;
the plurality of candidate optimized TCR sequences comprises amino acid sequences selected from the group consisting of SEQ ID NOs: 5-31;
the TP53 R273H-derived 9-mer peptide is selected from the group consisting of SEQ ID NOs: 32-33, or an alanine-substituted variant thereof;
the alanine-substituted variant comprises a single alanine substitution at any one amino acid position of the TP53 R273H-derived 9-mer peptide sequence set forth in SEQ ID NO: 32 or SEQ ID NO: 33;
the single alanine-substituted variant of the peptide sequence set forth in SEQ ID NO: 32 is selected from the group consisting of SEQ ID NOs: 34-41; and
the single alanine-substituted variant of the peptide sequence set forth in SEQ ID NO: 33 is selected from the group consisting of SEQ ID NOs: 42-49;
(b) generating a plurality of candidate TCE constructs by incorporating the candidate optimized TCR sequences generated in step (a) into TCE constructs;
wherein each candidate TCE construct comprises:
(b1) a TCR-derived binding domain comprising the candidate optimized TCR sequence in a single-chain TCR (sTCR) format or a stapled single-chain TCR (spTCR) format, wherein the TCR-derived binding domain comprises a variable α chain region (α-TCR) and a variable β chain region (β-TCR);
(b2) an anti-CD3 binding domain capable of binding to a CD3 complex expressed on a T cell, wherein the anti-CD3 binding domain comprises a variable heavy chain region (α-anti-CD3) and a variable light chain region (β-anti-CD3) arranged in a single-chain variable fragment (scFv) format or a stapled single-chain variable fragment (spFv) format; and
(b3) an Fc domain comprises a first Fc polypeptide fragment and a second Fc polypeptide fragment configured in a knob-into-hole heterodimerization arrangement, in which an N-terminus of the second Fc polypeptide fragment is linked to a C-terminus of the first Fc polypeptide fragment by a peptide linker consisting of a 30-amino-acid glycine-serine sequence comprising five repeats of a G4S pentapeptide;
wherein each candidate TCE construct comprises the domains arranged, from N-terminus to C-terminus, in an order selected from the group consisting of:
the TCR-derived binding domain, the anti-CD3 binding domain, and the Fc domain; or
the TCR-derived binding domain, the Fc domain, and the anti-CD3 binding domain;
(c) evaluating each candidate TCE construct based on three criteria to identify a TCE specifically recognizing the pMHC, wherein the TCE specifically recognizing the pMHC comprising HLA-C*01:02 presenting the TP53 R273H-derived 9-mer peptide is encoded by a nucleic acid or is a polypeptide;
wherein the three criteria comprise:
(c1) evaluating binding of the candidate TCE constructs to the pMHC comprising HLA-C*01:02 presenting the TP53 R273H-derived 9-mer peptide, wherein evaluating binding comprises:
(A) providing cells expressing HLA-C01:02;
(B) pulsing the cells with the TP53 R273H-derived 9-mer peptide to form a pHLA-C01:02/TP53 R273H peptide complex;
(C) incubating the pHLA-C01:02/TP53 R273H peptide-expressing cells with the candidate TCE constructs;
(D) stabilizing interactions between the candidate TCE constructs and the pHLA complexes by cross-linking with formaldehyde at a final concentration of 1% (v/V);
(E) quenching the cross-linking reaction by contracting the cells with Tris-HCl at pH 8.0 at a final concentration of 0.75 M;
(F) washing the cells to remove unbound the candidate TCE constructs and reagents;
(G) detecting TCE-bound cells using an Fc-specific detection reagent; and
(H) quantifying TCE binding to the pHLA-expressing cells by flow cytometry;
(c2) evaluating functional activation and T cell-mediated cytotoxicity induced by the candidate TCE constructs by co-culturing immune effector cells with antigen-presenting tumor cells in the presence of the candidate TCE constructs;
wherein T cell activation is evaluated by detecting expression of one or more markers selected from the group consisting of CD69, perforin, and granzyme B;
wherein T cell-mediated cytotoxicity is evaluated by measuring interferon-gamma (IFN-γ) release and/or assessing tumor cell killing;
(c3) evaluating reduced off-target reactivity relative to binding to the pMHC comprising HLA-C01:02 presenting the TP53 R273H-derived 9-mer peptide.

2. The method of claim 1, wherein the cells expressing the pMHC comprising HLA-C*01:02 presenting the TP53 R273H-derived 9-mer peptide comprise antigen-presenting tumor cells;
wherein the antigen-presenting tumor cells are derived from a tumor cell line selected from the group consisting of SW620 colorectal carcinoma cells, H520 lung squamous carcinoma cells, and MDA-MB-157 breast carcinoma cells.

3. The method of claim 1, wherein each candidate TCE construct comprises, from N-terminus to C-terminus:
the T cell receptor-derived binding domain in the sTCR format or the spTCR format;
the anti-CD3 binding domain in the scFv format; and
the Fc domain.

4. The method of claim 3, wherein the domain order of each candidate TCE construct, from N-terminus to C-terminus, is selected from the group consisting of:
(i) α-TCR, β-TCR, α-anti-CD3, β-anti-CD3, the first Fc polypeptide fragment comprising a knob mutation, and the second Fc polypeptide fragment comprising a hole mutation;
(ii) α-TCR, β-TCR, α-anti-CD3, β-anti-CD3, the first Fc polypeptide fragment comprising a hole mutation, and the second Fc polypeptide fragment comprising a knob mutation;
(iii) β-TCR, α-TCR, α-anti-CD3, β-anti-CD3, the first Fc polypeptide fragment comprising a knob mutation, and the second Fc polypeptide fragment comprising a hole mutation;
(iv) β-TCR, α-TCR, α-anti-CD3, β-anti-CD3, the first Fc polypeptide fragment comprising a hole mutation, and the second Fc polypeptide fragment comprising a knob mutation;
(v) α-TCR, β-TCR, β-anti-CD3, α-anti-CD3, the first Fc polypeptide fragment comprising a knob mutation, and the second Fc polypeptide fragment comprising a hole mutation;
(vi) α-TCR, β-TCR, β-anti-CD3, α-anti-CD3, the first Fc polypeptide fragment comprising a hole mutation, and the second Fc polypeptide fragment comprising a knob mutation;
(vii) β-TCR, α-TCR, β-anti-CD3, α-anti-CD3, the first Fc polypeptide fragment comprising a knob mutation, and the second Fc polypeptide fragment comprising a hole mutation; and
(viii) β-TCR, α-TCR, β-anti-CD3, α-anti-CD3, the first Fc polypeptide fragment comprising a hole mutation, and the second Fc polypeptide fragment comprising a knob mutation.

5. The method of claim 1, wherein each candidate TCE construct comprises, from N-terminus to C-terminus:
the T cell receptor-derived binding domain in the sTCR format or the spTCR format;
the Fc domain; and
the anti-CD3 binding domain in the scFv format.

6. The method of claim 5, wherein the domain order of each candidate TCE construct, from N-terminus to C-terminus, is selected from the group consisting of:
(i′) α-TCR, β-TCR, the first Fc polypeptide fragment comprising a knob mutation, the second Fc polypeptide fragment comprising a hole mutation, α-anti-CD3, and β-anti-CD3;
(ii′) α-TCR, β-TCR, the first Fc polypeptide fragment comprising a hole mutation, the second Fc polypeptide fragment comprising a knob mutation, α-anti-CD3, and β-anti-CD3;
(iii′) β-TCR, α-TCR, the first Fc polypeptide fragment comprising a knob mutation, the second Fc polypeptide fragment comprising a hole mutation, α-anti-CD3, and β-anti-CD3;
(iv′) β-TCR, α-TCR, the first Fc polypeptide fragment comprising a hole mutation, the second Fc polypeptide fragment comprising a knob mutation, α-anti-CD3, and β-anti-CD3;
(v′) α-TCR, β-TCR, the first Fc polypeptide fragment comprising a knob mutation, the second Fc polypeptide fragment comprising a hole mutation, β-anti-CD3, and α-anti-CD3;
(vi′) α-TCR, β-TCR, the first Fc polypeptide fragment comprising a hole mutation, the second Fc polypeptide fragment comprising a knob mutation, β-anti-CD3, and α-anti-CD3;
(vii′) β-TCR, α-TCR, the first Fc polypeptide fragment comprising a knob mutation, the second Fc polypeptide fragment comprising a hole mutation, β-anti-CD3, and α-anti-CD3; and
(viii′) β-TCR, α-TCR, the first Fc polypeptide fragment comprising a hole mutation, the second Fc polypeptide fragment comprising a knob mutation, β-anti-CD3, and α-anti-CD3.

7. The method of claim 1, wherein the TCE specifically recognizing the pMHC comprising HLA-C*01:02 presenting the TP53 R273H-derived 9-mer peptide is the polypeptide comprising an amino acid sequence selected from the group consisting of SEQ ID NOs: 50-55.

8. The method of claim 1, wherein the TCE specifically recognizing the pMHC comprising HLA-C*01:02 presenting the TP53 R273H-derived 9-mer peptide is encoded by the nucleic acid comprising a nucleotide sequence selected from the group consisting of SEQ ID NOs: 56-61;
wherein the nucleic acid is DNA or RNA,
wherein the DNA is selected from the group consisting of circular plasmid DNA, linear double-stranded DNA, single-stranded DNA, or a chimeric nucleic acid comprising DNA and RNA, and
wherein the RNA is messenger RNA (mRNA).

9. An engineered T cell engager receptor (TCE) specifically recognizing a peptide-major histocompatibility complex (pMHC) comprising HLA-C*01:02 presenting a TP53 R273H-derived 9-mer peptide, wherein the engineered TCE is encoded by a nucleic acid comprising a nucleotide sequence selected from the group consisting of SEQ ID NOs: 56-61, and
wherein the engineered TCE is identified by a method comprising:
(a) generating a plurality of candidate optimized T cell receptor (TCR) sequences by modifying a TCR sequence capable of recognizing the pMHC comprising HLA-C*01:02 presenting the TP53 R273H-derived 9-mer peptide;
wherein the TCR sequence is selected from the group consisting of SEQ ID NOS: 1-4;
the plurality of candidate optimized TCR sequences comprises amino acid sequences selected from the group consisting of SEQ ID NOs: 5-31;
the TP53 R273H-derived 9-mer peptide is selected from the group consisting of SEQ ID NOs: 32-33, or an alanine-substituted variant thereof;
the alanine-substituted variant comprises a single alanine substitution at any one amino acid position of the TP53 R273H-derived 9-mer peptide sequence set forth in SEQ ID NO: 32 or SEQ ID NO: 33;
the single alanine-substituted variant of the peptide sequence set forth in SEQ ID NO: 32 is selected from the group consisting of SEQ ID NOs: 34-41; and
the single alanine-substituted variant of the peptide sequence set forth in SEQ ID NO: 33 is selected from the group consisting of SEQ ID NOs: 42-49;
(b) generating a plurality of candidate TCE constructs by incorporating the candidate optimized TCR sequences generated in step (a) into TCE constructs;
wherein each candidate TCE construct comprises:
(b1) a TCR-derived binding domain comprising the candidate optimized TCR sequence in a single-chain TCR (sTCR) format or a stapled single-chain TCR (spTCR) format, wherein the TCR-derived binding domain comprises a variable α chain region (α-TCR) and a variable β chain region (β-TCR);
(b2) an anti-CD3 binding domain capable of binding to a CD3 complex expressed on a T cell, wherein the anti-CD3 binding domain comprises a variable heavy chain region (α-anti-CD3) and a variable light chain region (β-anti-CD3) arranged in a single-chain variable fragment (scFv) format or a stapled single-chain variable fragment (spFv) format; and
(b3) an Fc domain comprises a first Fc polypeptide fragment and a second Fc polypeptide fragment configured in a knob-into-hole heterodimerization arrangement, in which an N-terminus of the second Fc polypeptide fragment is linked to a C-terminus of the first Fc polypeptide fragment by a peptide linker consisting of a 30-amino-acid glycine-serine sequence comprising five repeats of a G4S pentapeptide;
wherein each candidate TCE construct comprises the domains arranged, from N-terminus to C-terminus, in an order selected from the group consisting of:
the TCR-derived binding domain, the anti-CD3 binding domain, and the Fc domain; or
the TCR-derived binding domain, the Fc domain, and the anti-CD3 binding domain;
(c) evaluating each candidate TCE construct based on three criteria to identify a TCE specifically recognizing the pMHC, wherein the TCE specifically recognizing the pMHC comprising HLA-C*01:02 presenting the TP53 R273H-derived 9-mer peptide is encoded by a nucleic acid or is a polypeptide;
wherein the three criteria comprise:
(c1) evaluating binding of the candidate TCE constructs to the pMHC comprising HLA-C*01:02 presenting the TP53 R273H-derived 9-mer peptide, wherein evaluating binding comprises:
(A) providing cells expressing HLA-C01:02;
(B) pulsing the cells with the TP53 R273H-derived 9-mer peptide to form a pHLA-C01:02/TP53 R273H peptide complex;
(C) incubating the pHLA-C01:02/TP53 R273H peptide-expressing cells with the candidate TCE constructs;
(D) stabilizing interactions between the candidate TCE constructs and the pHLA complexes by cross-linking with formaldehyde at a final concentration of 1% (v/v);
(E) quenching the cross-linking reaction by contracting the cells with Tris-HCl at pH 8.0 at a final concentration of 0.75 M;
(F) washing the cells to remove unbound the candidate TCE constructs and reagents;
(G) detecting TCE-bound cells using an Fc-specific detection reagent; and
(H) quantifying TCE binding to the pHLA-expressing cells by flow cytometry;
(c2) evaluating functional activation and T cell-mediated cytotoxicity induced by the candidate TCE constructs by co-culturing immune effector cells with antigen-presenting tumor cells in the presence of the candidate TCE constructs;
wherein T cell activation is evaluated by detecting expression of one or more markers selected from the group consisting of CD69, perforin, and granzyme B;
wherein T cell-mediated cytotoxicity is evaluated by measuring interferon-gamma (IFN-γ) release and/or assessing tumor cell killing;
(c3) evaluating reduced off-target reactivity relative to binding to the pMHC comprising HLA-C01:02 presenting the TP53 R273H-derived 9-mer peptide.

10. The engineered TCE of claim 9, wherein the cells expressing the pMHC comprising HLA-C*01:02 presenting the TP53 R273H-derived 9-mer peptide comprise antigen-presenting tumor cells;
wherein the antigen-presenting tumor cells are derived from a tumor cell line selected from the group consisting of SW620 colorectal carcinoma cells, H520 lung squamous carcinoma cells, and MDA-MB-157 breast carcinoma cells.

11. The engineered TCE of claim 9, wherein each candidate TCE construct comprises, from N-terminus to C-terminus:
the T cell receptor-derived binding domain in the sTCR format or the spTCR format;
the anti-CD3 binding domain in the scFv format; and
the Fc domain.

12. The engineered TCE of claim 11, wherein the domain order of each candidate TCE construct, from N-terminus to C-terminus, is selected from the group consisting of:
(i) α-TCR, β-TCR, α-anti-CD3, β-anti-CD3, the first Fc polypeptide fragment comprising a knob mutation, and the second Fc polypeptide fragment comprising a hole mutation;
(ii) α-TCR, β-TCR, α-anti-CD3, β-anti-CD3, the first Fc polypeptide fragment comprising a hole mutation, and the second Fc polypeptide fragment comprising a knob mutation;
(iii) β-TCR, α-TCR, α-anti-CD3, β-anti-CD3, the first Fc polypeptide fragment comprising a knob mutation, and the second Fc polypeptide fragment comprising a hole mutation;
(iv) β-TCR, α-TCR, α-anti-CD3, β-anti-CD3, the first Fc polypeptide fragment comprising a hole mutation, and the second Fc polypeptide fragment comprising a knob mutation;
(v) α-TCR, β-TCR, β-anti-CD3, α-anti-CD3, the first Fc polypeptide fragment comprising a knob mutation, and the second Fc polypeptide fragment comprising a hole mutation;
(vi) α-TCR, β-TCR, β-anti-CD3, α-anti-CD3, the first Fc polypeptide fragment comprising a hole mutation, and the second Fc polypeptide fragment comprising a knob mutation;
(vii) β-TCR, α-TCR, β-anti-CD3, α-anti-CD3, the first Fc polypeptide fragment comprising a knob mutation, and the second Fc polypeptide fragment comprising a hole mutation; and
(viii) β-TCR, α-TCR, β-anti-CD3, α-anti-CD3, the first Fc polypeptide fragment comprising a hole mutation, and the second Fc polypeptide fragment comprising a knob mutation.

13. The engineered TCE of claim 9, wherein each candidate TCE construct comprises, from N-terminus to C-terminus:
the T cell receptor-derived binding domain in the sTCR format or the spTCR format;
the Fc domain; and
the anti-CD3 binding domain in the scFv format.

14. The engineered TCE of claim 13, wherein the domain order of each candidate TCE construct, from N-terminus to C-terminus, is selected from the group consisting of:
(i′) α-TCR, β-TCR, the first Fc polypeptide fragment comprising a knob mutation, the second Fc polypeptide fragment comprising a hole mutation, α-anti-CD3, and β-anti-CD3;
(ii′) α-TCR, β-TCR, the first Fc polypeptide fragment comprising a hole mutation, the second Fc polypeptide fragment comprising a knob mutation, α-anti-CD3, and β-anti-CD3;
(iii′) β-TCR, α-TCR, the first Fc polypeptide fragment comprising a knob mutation, the second Fc polypeptide fragment comprising a hole mutation, α-anti-CD3, and β-anti-CD3;
(iv′) β-TCR, α-TCR, the first Fc polypeptide fragment comprising a hole mutation, the second Fc polypeptide fragment comprising a knob mutation, α-anti-CD3, and β-anti-CD3;
(v′) α-TCR, β-TCR, the first Fc polypeptide fragment comprising a knob mutation, the second Fc polypeptide fragment comprising a hole mutation, β-anti-CD3, and α-anti-CD3;
(vi′) α-TCR, β-TCR, the first Fc polypeptide fragment comprising a hole mutation, the second Fc polypeptide fragment comprising a knob mutation, β-anti-CD3, and α-anti-CD3;
(vii′) β-TCR, α-TCR, the first Fc polypeptide fragment comprising a knob mutation, the second Fc polypeptide fragment comprising a hole mutation, β-anti-CD3, and α-anti-CD3; and
(viii′) β-TCR, α-TCR, the first Fc polypeptide fragment comprising a hole mutation, the second Fc polypeptide fragment comprising a knob mutation, β-anti-CD3, and α-anti-CD3.

15. The engineered TCE of claim 9, wherein the nucleic acid encoding the engineered TCE is comprised within a vector or an expression cassette;
wherein the vector or expression cassette comprises one or more regulatory elements operably linked to the nucleic acid;
the regulatory elements being selected from the group consisting of a promoter, an enhancer, a transcription termination signal, a polyadenylation signal, and combinations thereof.

16. An engineered T cell engager receptor (TCE) specifically recognizing a peptide-major histocompatibility complex (pMHC) comprising HLA-C*01:02 presenting a TP53 R273H-derived 9-mer peptide, wherein the engineered TCE is a polypeptide comprising an amino acid sequence selected from the group consisting of SEQ ID NOs: 50-55, and
wherein the engineered TCE is identified by a method comprising:
(a) generating a plurality of candidate optimized T cell receptor (TCR) sequences by modifying a TCR sequence capable of recognizing the pMHC comprising HLA-C*01:02 presenting the TP53 R273H-derived 9-mer peptide;
wherein the TCR sequence is selected from the group consisting of SEQ ID NOS: 1-4;
the plurality of candidate optimized TCR sequences comprises amino acid sequences selected from the group consisting of SEQ ID NOs: 5-31;
the TP53 R273H-derived 9-mer peptide is selected from the group consisting of SEQ ID NOs: 32-33, or an alanine-substituted variant thereof;
the alanine-substituted variant comprises a single alanine substitution at any one amino acid position of the TP53 R273H-derived 9-mer peptide sequence set forth in SEQ ID NO: 32 or SEQ ID NO: 33;
the single alanine-substituted variant of the peptide sequence set forth in SEQ ID NO: 32 is selected from the group consisting of SEQ ID NOs: 34-41; and
the single alanine-substituted variant of the peptide sequence set forth in SEQ ID NO: 33 is selected from the group consisting of SEQ ID NOs: 42-49;
(b) generating a plurality of candidate TCE constructs by incorporating the candidate optimized TCR sequences generated in step (a) into TCE constructs;
wherein each candidate TCE construct comprises:
(b1) a TCR-derived binding domain comprising the candidate optimized TCR sequence in a single-chain TCR (sTCR) format or a stapled single-chain TCR (spTCR) format, wherein the TCR-derived binding domain comprises a variable α chain region (α-TCR) and a variable β chain region (β-TCR);
(b2) an anti-CD3 binding domain capable of binding to a CD3 complex expressed on a T cell, wherein the anti-CD3 binding domain comprises a variable heavy chain region (α-anti-CD3) and a variable light chain region (β-anti-CD3) arranged in a single-chain variable fragment (scFv) format or a stapled single-chain variable fragment (spFv) format; and
(b3) an Fc domain comprises a first Fc polypeptide fragment and a second Fc polypeptide fragment configured in a knob-into-hole heterodimerization arrangement, in which an N-terminus of the second Fc polypeptide fragment is linked to a C-terminus of the first Fc polypeptide fragment by a peptide linker consisting of a 30-amino-acid glycine-serine sequence comprising five repeats of a G4S pentapeptide;
wherein each candidate TCE construct comprises the domains arranged, from N-terminus to C-terminus, in an order selected from the group consisting of:
the TCR-derived binding domain, the anti-CD3 binding domain, and the Fc domain; or
the TCR-derived binding domain, the Fc domain, and the anti-CD3 binding domain;
(c) evaluating each candidate TCE construct based on three criteria to identify a TCE specifically recognizing the pMHC, wherein the TCE specifically recognizing the pMHC comprising HLA-C*01:02 presenting the TP53 R273H-derived 9-mer peptide is encoded by a nucleic acid or is a polypeptide;
wherein the three criteria comprise:
(c1) evaluating binding of the candidate TCE constructs to the pMHC comprising HLA-C*01:02 presenting the TP53 R273H-derived 9-mer peptide, wherein evaluating binding comprises:
(A) providing cells expressing HLA-C01:02;
(B) pulsing the cells with the TP53 R273H-derived 9-mer peptide to form a pHLA-C01:02/TP53 R273H peptide complex;
(C) incubating the pHLA-C01:02/TP53 R273H peptide-expressing cells with the candidate TCE constructs;
(D) stabilizing interactions between the candidate TCE constructs and the pHLA complexes by cross-linking with formaldehyde at a final concentration of 1% (v/V);
(E) quenching the cross-linking reaction by contracting the cells with Tris-HCl at pH 8.0 at a final concentration of 0.75 M;
(F) washing the cells to remove unbound the candidate TCE constructs and reagents;
(G) detecting TCE-bound cells using an Fc-specific detection reagent; and
(H) quantifying TCE binding to the pHLA-expressing cells by flow cytometry;
(c2) evaluating functional activation and T cell-mediated cytotoxicity induced by the candidate TCE constructs by co-culturing immune effector cells with antigen-presenting tumor cells in the presence of the candidate TCE constructs;
wherein T cell activation is evaluated by detecting expression of one or more markers selected from the group consisting of CD69, perforin, and granzyme B;
wherein T cell-mediated cytotoxicity is evaluated by measuring interferon-gamma (IFN-γ) release and/or assessing tumor cell killing;
(c3) evaluating reduced off-target reactivity relative to binding to the pMHC comprising HLA-C01:02 presenting the TP53 R273H-derived 9-mer peptide.

17. The engineered TCE of claim 16, wherein the cells expressing the pMHC comprising HLA-C*01:02 presenting the TP53 R273H-derived 9-mer peptide comprise antigen-presenting tumor cells;
wherein the antigen-presenting tumor cells are derived from a tumor cell line selected from the group consisting of SW620 colorectal carcinoma cells, H520 lung squamous carcinoma cells, and MDA-MB-157 breast carcinoma cells.

18. The engineered TCE of claim 16, wherein each candidate TCE construct comprises, from N-terminus to C-terminus, the T cell receptor-derived binding domain in the sTCR format or the spTCR format, the anti-CD3 binding domain in the scFv format, and the Fc domain;
wherein the domain order of each candidate TCE construct, from N-terminus to C-terminus, is selected from the group consisting of:
(i) α-TCR, β-TCR, α-anti-CD3, β-anti-CD3, the first Fc polypeptide fragment comprising a knob mutation, and the second Fc polypeptide fragment comprising a hole mutation;
(ii) α-TCR, β-TCR, α-anti-CD3, β-anti-CD3, the first Fc polypeptide fragment comprising a hole mutation, and the second Fc polypeptide fragment comprising a knob mutation;
(iii) β-TCR, α-TCR, α-anti-CD3, β-anti-CD3, the first Fc polypeptide fragment comprising a knob mutation, and the second Fc polypeptide fragment comprising a hole mutation;
(iv) β-TCR, α-TCR, α-anti-CD3, β-anti-CD3, the first Fc polypeptide fragment comprising a hole mutation, and the second Fc polypeptide fragment comprising a knob mutation;
(v) α-TCR, β-TCR, β-anti-CD3, α-anti-CD3, the first Fc polypeptide fragment comprising a knob mutation, and the second Fc polypeptide fragment comprising a hole mutation;
(vi) α-TCR, β-TCR, β-anti-CD3, α-anti-CD3, the first Fc polypeptide fragment comprising a hole mutation, and the second Fc polypeptide fragment comprising a knob mutation;
(vii) β-TCR, α-TCR, β-anti-CD3, α-anti-CD3, the first Fc polypeptide fragment comprising a knob mutation, and the second Fc polypeptide fragment comprising a hole mutation; and
(viii) β-TCR, α-TCR, β-anti-CD3, α-anti-CD3, the first Fc polypeptide fragment comprising a hole mutation, and the second Fc polypeptide fragment comprising a knob mutation.

19. The engineered TCE of claim 16, wherein each candidate TCE construct comprises, from N-terminus to C-terminus, the T cell receptor-derived binding domain in the sTCR format or the spTCR format, the Fc domain, and the anti-CD3 binding domain in the scFv format;
wherein the domain order of each candidate TCE construct, from N-terminus to C-terminus, is selected from the group consisting of:
(i′) α-TCR, β-TCR, the first Fc polypeptide fragment comprising a knob mutation, the second Fc polypeptide fragment comprising a hole mutation, α-anti-CD3, and β-anti-CD3;
(ii′) α-TCR, β-TCR, the first Fc polypeptide fragment comprising a hole mutation, the second Fc polypeptide fragment comprising a knob mutation, α-anti-CD3, and β-anti-CD3;
(iii′) β-TCR, α-TCR, the first Fc polypeptide fragment comprising a knob mutation, the second Fc polypeptide fragment comprising a hole mutation, α-anti-CD3, and β-anti-CD3;
(iv′) β-TCR, α-TCR, the first Fc polypeptide fragment comprising a hole mutation, the second Fc polypeptide fragment comprising a knob mutation, α-anti-CD3, and β-anti-CD3;
(v′) α-TCR, β-TCR, the first Fc polypeptide fragment comprising a knob mutation, the second Fc polypeptide fragment comprising a hole mutation, β-anti-CD3, and α-anti-CD3;
(vi′) α-TCR, β-TCR, the first Fc polypeptide fragment comprising a hole mutation, the second Fc polypeptide fragment comprising a knob mutation, β-anti-CD3, and α-anti-CD3;
(vii′) β-TCR, α-TCR, the first Fc polypeptide fragment comprising a knob mutation, the second Fc polypeptide fragment comprising a hole mutation, β-anti-CD3, and α-anti-CD3; and
(viii′) β-TCR, α-TCR, the first Fc polypeptide fragment comprising a hole mutation, the second Fc polypeptide fragment comprising a knob mutation, β-anti-CD3, and α-anti-CD3.

20. The engineered TCE of claim 16, wherein a nucleic acid encoding the polypeptide comprising the engineered TCE is comprised within a vector or an expression cassette;
wherein the vector or expression cassette comprises one or more regulatory elements operably linked to the nucleic acid;
the regulatory elements being selected from the group consisting of a promoter, an enhancer, a transcription termination signal, a polyadenylation signal, and combinations thereof.


SEQUENCE LISTING (125 sequences)

SEQ ID NO: 1 - protein, 457 aa, Homo sapiens
QQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGSGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPGGGGSGGGGSGGGGSEAAVTQSPRNKVAVTGGKVTLQCAQDMNNHNNMYWYRQDTGHGLRLIHYSASEGTTEKGDIPDGYKASRPSQENFSLILELATPSQTSVYFCATKSSLTYEQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 2 - DNA, 1371 bp, Homo sapiens
  source 1..1371: mol_type = genomic DNA
cagcagaaagtgcaacaaagtcctgagagcctcatcgttcccgaaggggggatggcctcccttaattgtacaagcagtaactcaatgttcgatttttggtggtatcgccagcactcaggaaagtcaccaaagatgctgatgtccatttcttccatcaaagacaagaacgaggaggggagattcactgtgcatctgaataaggcgagcctgcacacctctctgcatatacgagattctcaaccaagtgatagcgctttgtacctgtgcgccgctagcgacaatgacatgcggtttggctctggcacgaagctcatcatcaaacccgatattcagaaccctgatcctgccgtttaccagctcagagactctaaatctagcgataaaagcgtatgccttttcactgattttgacagtcagacgaacgtgagtcagagcaaagacagcgatgtttacattacagacaaaacagtgctggacatgaggagtatggatttcaagagcaattctgctgtggcatggagtaataagtccgacttcgcatgcgccaacgccttcaataattccattattcctgaagacacgttctttccatcacccggagggggaggaagtgggggcggaggttcagggggagggggtagcgaggcagcggtgacacaaagccccagaaataaagtagccgtgacaggggggaaagtgaccctgcagtgtgcccaggacatgaataatcataacaatatgtactggtatcgacaggacacaggacacggactgagacttattcactacagtgccagtgaaggtactacagagaaaggggacattcccgatggctataaagcgagcaggccgagtcaggagaattttagtctgatcctggaactggcaacaccatctcagaccagcgtgtacttttgcgctaccaagtcctcactgacatacgaacagtactttggcccaggaaccagactgaccgtcttggaggatttgaacaaagtctttccaccagaagtggccgtttttgagccttccgaggccgagattagtcatactcaaaaagcaactctggtatgcctggccacaggattcttcccagatcacgtcgagctgtcttggtgggtgaacggcaaggaagtacattctggtgtgagcactgatcctcaacccctcaaagaacaacctgccctgaacgactctaggtattgcctgagttcccgcttgagggtctctgctaccttctggcagaatcctagaaatcacttccggtgccaagtgcaattctatggcctgtctgagaatgacgagtggacccaggacagagctaaaccggtcacccaaattgtctctgccgaggcttggggcagggcagac

SEQ ID NO: 3 - protein, 238 aa, Homo sapiens
VTQSPRNKVAVTGGKVTLQCAQDMNNHNNMYWYRQDTGCGLRLIHYSASEGTTEKGDIPDGYKASRPSQENFSLILELATPSQTSVYFCATKSSLTYEQYFGPGTRLTVLEGGGGSGGGSCPPCGGGGSQQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGCGTKLIIKPD

SEQ ID NO: 4 - DNA, 714 bp, Homo sapiens
  source 1..714: mol_type = genomic DNA
gttacccagagcccccgaaataaagtggctgtgacggggggtaaagtgacactgcagtgtgctcaggacatgaacaaccataacaatatgtactggtatagacaggacaccggctgcggtttgcggctcattcactatagtgcctctgaagggacaacagagaaaggcgacataccggacggctacaaggccagccgcccttcccaagagaattttagtttgatcctggagctggcgaccccttcacagacaagcgtctatttttgtgcgaccaagagttccctgacatatgagcagtacttcggtccaggaactaggctcactgttctggaaggtggaggaggaagcgggggtgggtcttgtcccccttgtggagggggcggatcccagcagaaagttcagcaatctcccgaaagtctcatcgtgcctgagggcggaatggcttccctcaattgtaccagttccaacagcatgtttgacttctggtggtaccggcaacattcaggcaagtccccaaaaatgttgatgagcattagctctattaaagacaaaaacgaagagggccgcttcaccgtacacctcaacaaggctagtctccatacctccctccatattagagactcccaaccgagtgattcagccctctacctttgtgctgcatcagacaatgacatgcggtttggatgtggaacaaagttgatcatcaaacccgat

SEQ ID NO: 5 - protein, 238 aa, Homo sapiens
VTQSPRNLVAVTGGKVTLQCAQDRNPHNNMYWYRIDTGQGLRLIHYSASEGTTEKGDIPDGYKASRPSQENFSLILESATPSQTSVYFCATKSSLTYEQYFGPGTRLTVLEGGGGSGGGSGPPSGGGGSQQKVQQSPESLIVPEGGMASLNCTSSNSMFDFWWYRQHSGKSPKMLMSISSIKDKNEEGRFTVHLNKASSHTSLHIRDSQPSDSALYWCAASDNDMRFGCGTKLIIKPD

SEQ ID NO: 6 - protein, 476 aa, Homo sapiens
VTQSPRNLVAVTGGKVTLQCAQDRNNHNNMYWYRIDTGHGLRLIHYSASVITTEKGDIPDGYKASRPSSENFSLILESATPSQTSVYFCATSSDSTAEQYFGPGTRLTVLEGGGGSGGGSCPPCGGGGSQQKVEQSPESAVVPEGGMASLNCTSSNSMFDFWWYRQISGKGPKMLMSISSIKDKNEEGRFTVHLNKASSQTSLHIRGSQPSDSALYWCAASDNFMRFGQGTKLIIKPDVTQSPRNLVAVTGGKVTLQCAQDRNNHNNMYWYRIDTGHGLRLIHYSASVITTEKGDIPDGYKASRPSSENFSLILESATPSQTSVYFCATSSDSTAEQYFGPGTRLTVLEGGGGSGGGSCPPCGGGGSQQKVQQSPESLIVPEGGMASLNCTSSNSMFDFWWYRQHSGKSPKMLMSISSIKDKNEEGRFTVHLNKASSHTSLHIRDSQPSDSALYWCAASDNDMRFGCGTKLIIKPD

SEQ ID NO: 7 - protein, 238 aa, Homo sapiens
VTQSPRNLVAVTGGKVTLQCAQDRNNHNNMYWYRIDTGHGLRLIHYSASVITTEKGDIPDGYKASRPSSENFSLILESATPSQTSVYFCATSSDSTAEQYFGPGTRLTVLEGGGGSGGGSCPPCGGGGSQQKVQQSPESLIVPEGGMASLNCTSSNSMFDFWWYRQHSGKSPKMLMSISSIKDKNEEGRFTVHLNKASSHTSLHIRDSQPSDSALYWCAASDNDMRFGCGTKLIIKPD

SEQ ID NO: 8 - DNA, 714 bp, Homo sapiens
  source 1..714: mol_type = genomic DNA
gtgacacagtctccccgaaatctggttgctgtcacaggcgggaaagtcactctccaatgtgcgcaagaccgaaatccacacaataacatgtattggtatagaattgacactggacagggacttaggcttatccattactcagcctctgagggaaccactgaaaagggagatattccagacggctacaaggcgagtcgcccttcccaggagaacttctctctgatcctcgagtcagccacccccagccagacgagcgtgtacttttgtgccacaaaatcaagcctgacatatgaacagtattttggccccggcaccaggcttactgttttggagggtggaggagggagcggtggaggttctgggcctcctagcgggggcggcggctcacagcaaaaggtgcagcagagtccggagtcattgattgtgcccgagggtgggatggcctccctgaattgcacgagttccaactcaatgtttgacttctggtggtaccgacaacattccggcaaaagcccgaaaatgctgatgtccatcagctcaatcaaagacaaaaatgaggagggcaggtttacagtccatctgaacaaagccagcagtcacacctctctgcatatccgcgacagtcaacctagtgattccgccctctattggtgcgccgcctccgacaatgacatgagatttgggtgtgggactaagctgattataaagcccgac

SEQ ID NO: 9 - DNA, 714 bp, Homo sapiens
  source 1..714: mol_type = genomic DNA
gtgacccagtcaccccgcaatctcgtagctgtcacaggtggcaaggtaaccctgcagtgcgcacaggaccgaaataatcacaataacatgtactggtatcggatcgacactggccacgggctgagattgatccactacagcgcttctgtgattactaccgagaagggcgacatccctgatggctacaaagccagccgcccttcatcagagaacttcagtctgattctggaatctgctacaccatcacagacgtctgtttatttctgcgccaccagcagcgactctacggcagagcagtattttggccctggaacaagattgacggtgctcgaaggtggaggggggtcaggcggcggatcttgtccaccgtgcgggggtggcggaagccagcagaaagttgagcagtctccggaatctgctgtggtgcctgagggcggcatggcaagcctgaattgcacaagctctaactccatgttcgacttctggtggtatcggcagatcagtggaaaggggcctaagatgctcatgtctatcagttcaatcaaggacaagaacgaagaggggcgcttcaccgttcacctgaacaaggcaagcagccaaacttccctgcacattcgcggatctcagccgagcgattccgccctgtactggtgcgcagcttctgataatttcatgcgcttcggtcagggaactaagttgataatcaaacccgat

SEQ ID NO: 10 - DNA, 714 bp, Homo sapiens
  source 1..714: mol_type = genomic DNA
gtcacacagagcccgaggaacttggtggctgtaacaggtgggaaggttacgctgcagtgtgctcaggatcggaataaccataacaatatgtactggtaccgaatcgacaccggtcatggactgcggctgatacactactccgcttcagtgatcacaacggaaaaaggagacatcccagacggttacaaggcaagccgcccctctagtgagaacttctctctcatcctggaaagcgccactcccagtcaaacgagtgtatatttctgtgcaacaagctccgactctaccgcagaacagtatttcggcccaggcaccagactgacagtcctggaaggaggaggagggtctggcgggggcagttgtccaccatgcgggggtggcggaagtcagcaaaaagtccagcagagcccagaatctttgatcgtccctgagggcgggatggcctctctcaattgtacctcatccaacagcatgttcgatttttggtggtacagacagcactctgggaaatcccctaagatgctgatgagcatcagcagcataaaggacaagaatgaagaaggacggtttacagttcatctcaacaaggcttcaagtcacacaagcctgcacatccgggattctcaaccgagcgattctgcactgtactggtgcgccgctagcgacaatgacatgcgcttcggttgtggcacaaagctgattatcaagcccgat

SEQ ID NO: 11 - protein, 238 aa, synthetic construct
VTQSPRNLVAVTGGAVTLQCAQNMNNHNNMYWYRQDTGQGLRLIHYSASEDTTEKGDIPDGYKASRPSQENFSLIIQPATPSQTSVYFCATKSSGTYEQYFGPGTRLTVLEGGGGSGGGSGPAEGGGGSQQKVQQSPESFIVPEGGSISLNCTSSNSMFDFWWYRQHSGKSPKMLMSISSIKDKNEEGRFTVHLNKASRHTSLHIRDSQPSDSALYLCAASDNDMRFGRGTKLIIKPD

SEQ ID NO: 12 - protein, 238 aa, synthetic construct
VTQSPRNKVAVTGGKVTLQCAQDMNNHNNMYWYRQDTGGGLRLIHYSASEGTTEKGDIPDGYKASRPSQENFSLILELATPSQTSVYFCATSSSLTYEQYFGHGTRLTVLEGGGGSGGGSAPPLGGGGSQQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGCGTKLIIKPD

SEQ ID NO: 13 - protein, 238 aa, synthetic construct
VTQSPRNKVAVTGGKVTLQCAQDMKNHNNMYWYRQDTGPGLRLIYYSASEGTTEKGDIPDGYKASRTSQENFSLILEEATPSQTSVYFCATKSSLTYEQYFGPGTRLNVLEGGGGSGGGSNPPSGGGGSQQNVQQSPESVIVPEGGSASVNCTSSNSSFDFWWYRQHSGKSPKMLMSISSIKDKNEEGRFTVHLNKASYHTSLHIRDSQPSDSALYYCAASDNDMRFGCGTKLIVKPD

SEQ ID NO: 14 - protein, 238 aa, synthetic construct
VTQSPRNKVAVTGGKVTIQCAQDMNNHNNMYWYRQDTGQGPRLIHYSYSEGTTEKGDIPDGYKASRPSQENFSLIIERTTPSQTSVYFCATKSSLTYGQTFGPGTRLTVLEGGGGSGGGSSPPGGGGGSQQKVQQSPESPIVPEGGSATLNCTSSGSMFDFWWYRQHSGKSPKMLMSISSISDKNEEGRFTVHLNKTSIHTSLHIRDSQPSDSALYLCAASDNDMRFGCGTKLIIKPD

SEQ ID NO: 15 - protein, 238 aa, synthetic construct
VTQSPREKVAVTGGKVTLQCAQDMNNHPNMYWYRQDTGKGPRLIHYSASEGQTEKGRIPDGYKASRPSQENFSLILESATPSQTSVYFCATKSFLTYEQYFGPGTRLTVLEGGGGSGGGSPPPGGGGGSQQTVQQSPESLIVPEGGAASLNCHSSNSMVDFWWYRQHSGKSPKRLMSISSIKDKNEEGRFTVHLNKASSHTSLHITDSQSSDSALYLCAASDNDMRFGCGTKLIIKPD

SEQ ID NO: 16 - protein, 238 aa, synthetic construct
VTQSPRDKVAVTGGKVTLQCAQDMNNHNNMYWYRQDTGAGLRLIHYSASEGTTEKGDIPDGYKASRPSQENFSLILEAATPSQTSVYFCATKSSLTVSQYFGTGTRLTVLEGGGGSGGGSSPPSGGGGSQQKVQQSPESVTVPEGGAASLNCTSSNSRFDFWWYRQHSGKSPKMLMSISSIKDQEEEGRFTVHDNKASRHTSLHIRKSQPSDSALYFCAASDNDIRFGCGTKLIIKPD

SEQ ID NO: 17 - protein, 238 aa, synthetic construct
VTQSPRNLVAVTGGKVTLQCAQSMNNHNNMYWYRQDTGKGLRLIHYSISEGTTEKGDIPDGYKASRPSQENFSLILESATPSQTSVYFCATKSSLTYEQYFGPGTRLTVLEGGGGSGGGSPPPAGGGGSQQSVQQSPESIIVPEGGMATLNCTSSESMFDFWWYRQHSGKSPKMLMSISSIKDKNEEGRFTVELNKASSHTSLHIRDSQPSDSALYLCAASDNDMRFGCGTKLIIKPD

SEQ ID NO: 18 - protein, 238 aa, synthetic construct
VTQSPRNKVAVTGGKVTLQCAQDMNNHNNMYWYRQDPGIGLRLIHYSASEGTTEKGDIPDGYKASRPSQENFSLILELATPSQTSVYFCATKSSLTYEQYFGPGTRLTVLEGGGGSGGGSPPPTGGGGSQQKVQQSPESMIVPEGGMASLNCTSSNSMFDFWWYRQHSGKSPKMLMSISSIKDKNEEGRFTVHLNKASLQTSLHIRDSQPSDSALYLCAASDNDMRFGCGTKLIIKPD

SEQ ID NO: 19 - protein, 238 aa, synthetic construct
VTQSPRNKVAVTGGKVTLQCAQTMNNHNNMYWYRQDTGQGLRLIHYSASEGTTEKGDIPDGYKASRPSQENFSLILELATPSQTSVYFCATKSSLTYEQYFGPGTRLTVLEGGGGSGGGSCPPCGGGGSQQVVQQSPESLIVPEGGMASLNCTSSNSMGDFWWYRQHSGKSPKMLMSISSIKDKNEEGRFTVHLNKASLHTSLHIRDSQPSDSALYLCAASDNDMRFGCGTKLIIKPD

SEQ ID NO: 20 - protein, 238 aa, synthetic construct
VTQSPRNKVAVTGGKVTLQCAQDMNNHNNMYWYRQDTGQGLRLIHYSASEGTTEKGDIPDGYKASRPSQENFSLILESATPSQTSVYFCATKSSLTYEQYFGPGTRLTVLEGGGGSGGGSGPPTGGGGSQQKVQQSPESRIVPEGGSASLNCTSSNSMFDFWWYRQHSGKSPKMLMSISSIKDKNEEGRFTVHLNKASQHTSLHIRDSQPSDSALYLCAASDNDMRFGCGTKLIIKPD

SEQ ID NO: 21 - protein, 238 aa, synthetic construct
VTQSPRNKVAVTGGKVTLQCAQDMNNHNNMYWYRQDTGPGLRLIHYSASEGTTEKGDIPDGYKASRPSQENFSLILELATPSQTSVYFCATKSSLTYEQYFGPGTRLTVLEGGGGSGGGSGPPCGGGGSQQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGCGTKLIIKPD

SEQ ID NO: 22 - protein, 238 aa, synthetic construct
VTQSPRNLVAVTGGKVTLQCAQDLNNHNNMYWYRQDTGPGLRLIHYSASEGTTEKGDIPDGYKASRPSQENFSLIIQNATPSQTSVYFCATKSSLTGEQYFSQGTRLTVLEGGGGSGGGSPPPSGGGGGQQKVQQSPESIIVPEGGTASLNCPSSNSMFDFWWYQQHSGKSPKMLMSISSIKDKNEEGRFTVHLNKASDHTSLHIRDSQPSDSAMYYCAASDSDMRFGCGTKLIIKPD

SEQ ID NO: 23 - protein, 238 aa, synthetic construct
VTQSPRNLVAVTGGKVTLQCAQDRNPHNNMYWYRIDTGQGLRLIHYSASEGTTEKGDIPDGYKASRPSQENFSLILESATPSQTSVYFCATKSSLTYEQYFGPGTRLTVLEGGGGSGGGSGPPGGGGGSQQKVQQSPESLIVPEGGMASLNCTSSNSIFDFWWYRQHSGKSPKMLMSISSIKDKNEEGRFTVHLNKASSHTSLHIRDSQPSDSALYWCAASDNDMRFGQGTKLIIKPD

SEQ ID NO: 24 - protein, 238 aa, synthetic construct
VTQSPRNLVAVTGGKVTLQCAQDVQPHNNMYWYRSDAGRGLRLIHYSAAEGTTEKGDIPDGYKASRPSQENFSLILESATPSQTSVYFCATKSSLTYEQYFGPGTRLTVLEGGGGSGGGSGPPGGGGGSQQKVQQSPESVIVPEGGMASLNCTSSNSMFDFWWYRQYSGKSPKMFMSISSIKDKNEEGRFTVVLNKSSSHTSLHIVDSQPSDSALYWCAASDNDMRFGQGTKLIIKPD

SEQ ID NO: 25 - protein, 238 aa, synthetic construct
VTQSPRNVVAVTGGRVTWQCAQDPNPHNNMYWYRIDTGGGLRLIHYSASEGTTEKGDIPDGYKASRPSQENFSLIIESASPSQTSVYFCATDSSSTYEQYFGPGTKLTVLEGGGGSGGGSGDPSGGGGSQQVVQQSPPSFIVPEGGAASVNCTSSNSMFDFYWYRQHSGKSPKMLMSISSIKDKNEEGRFTVHLNKASSHTSLHIRDSQPSDSALYFCAASDNDMRFGQGTKLIIKPD

SEQ ID NO: 26 - protein, 238 aa, synthetic construct
VTQSPRDLVPVTGGKVTLSCAQDRNPHNYMYWYRRDTGEGLRLIHYSASEGTTEKGDIPDGYKASRPSQENFSLIIESATPSQTSVYFCATKSSSTYEQYFGPGTRLTVLEGGGGSGGGSGPPEGGGGQQQKVQQSPESVSVPEGGMASLNCTSSNSMFDFWWYRQHSGKSPKMLMSISSIKDKMEEGRFTVHLNKASSTTSLHIKDSQPSDSALYWCAASDNDMRFGQGTKLIIKPD

SEQ ID NO: 27 - protein, 238 aa, synthetic construct
VTQSPRNLIAVTGGKVTLQCAQDRNPHNNMYWYRIDTGQGLRLIHYSASEGYTEKGDIPDGYKASRPSQENFSLILESATPSQTSVYFCATKSSLTYEQYFGAGTRLTVLEGGGGSGGGSGPPGGGGGSQQKVQQSPESLIVPEGGNASLNCTSSNSMFDFWWYRQHSGKSPKMLMSITSIKDKNEEGRFTVHLNKASSHTSLHIRDSQPSDSALYWCAASDGDMRFGQGTKLYIKPD

SEQ ID NO: 28 - protein, 238 aa, synthetic construct
VTQSPRNLVAVTGGKVTLQCAQDRNPHNNMYWYRIDTGLGLRLIHYSASEGTTEKGDIPDGYKASRPSQENFSLILESATPSQTSVYFCATKSSLTYEQYFGPGTRLTVLEGGGGSGGGSQPPPGGGGSQQKVQQSPESVIVPEGGMASLNCTSSNSMFDFFWYRQHSGKSPKMLMSISSIKDKNEEGRFTVHLNKASSHTSLHIRDSQPSDSALYWCAASDNDMRFGQGTKLIIKPD

SEQ ID NO: 29 - protein, 238 aa, synthetic construct
VTQSPRSVVAVTGGKVTLQCAQDRNPHNNMYWYRIDTGQGLRLIHYSASEGTTEKGDIPDGYKASRPSQENFSLIIISATPSQTSVYFCATKSSGTYEQYFGPGTRLTVLGGGGGSGGGSEPPPPGGGSQQKVQQSPESIIVPEGGMASLNCTSSNSMFDFWWYRQHSGKSPKMLMSISSIKDKNEEGRFTVHLNKASSTTSLHIRDSQPSDSALYWCAASDNDMRFGQGTKLIIIPD

SEQ ID NO: 30 - protein, 238 aa, synthetic construct
TTQSPRNLVAVPGGKVTLQCAQDRNPHNNMYWYRIDTGLGLRLIHYSASEGTTEKGDIPDGYKASRPSQENFSLILESATPSQTSVYFCATKSSLTYEQYFGPGTRLTVLPGGGGSGGGSGIPGGGGGSQQKVQQSPESVIVPEGGMISLNCTSSNSMFDFWWYRQHSGKSPKMLMSISSIKDKNEEGRFTVHINKASSHTSLHIRDSQPSDSALYWCAASDNHMRFGQGTKLIIKPD

SEQ ID NO: 31 - protein, 238 aa, synthetic construct
VTQSPRNLVAVTGHKVTLQCNQDRNPHNIMYWYRIDTGQGLRLIHYSASEGTTEKGDIPDGYKASRPSQENFSLILDSATPSQTSVYFCATKSSLTYEQYFGPGTRLTVLEGGGGSGGGSGPPGGGGGSQQKVQQSPESVIVPEGGLASLNCTSSNGMFDFWWYRQHSGKSPKMLMSISSIKDKNEEGRFTVSLNKASSHTSLHIRDSQPSDSALYWCAASDNDMRFGQGTKLVIKPD

SEQ ID NO: 32 - protein, 9 aa, Homo sapiens
SFEVHVCAC

SEQ ID NO: 33 - protein, 9 aa, synthetic construct
NSFEVHVCA

SEQ ID NO: 34 - protein, 9 aa, synthetic construct
AFEVHVCAC

SEQ ID NO: 35 - protein, 9 aa, synthetic construct
SAEVHVCAC

SEQ ID NO: 36 - protein, 9 aa, synthetic construct
SFAVHVCAC

SEQ ID NO: 37 - protein, 9 aa, synthetic construct
SFEAHVCAC

SEQ ID NO: 38 - protein, 9 aa, synthetic construct
SFEVAVCAC

SEQ ID NO: 39 - protein, 9 aa, synthetic construct
SFEVHACAC

SEQ ID NO: 40 - protein, 9 aa, synthetic construct
SFEVHVAAC

SEQ ID NO: 41 - protein, 9 aa, synthetic construct
SFEVHVCAA

SEQ ID NO: 42 - protein, 9 aa, synthetic construct
ASFEVHVCA

SEQ ID NO: 43 - protein, 9 aa, synthetic construct
NAFEVHVCA

SEQ ID NO: 44 - protein, 9 aa, synthetic construct
NSAEVHVCA

SEQ ID NO: 45 - protein, 9 aa, synthetic construct
NSFAVHVCA

SEQ ID NO: 46 - protein, 9 aa, synthetic construct
NSFEAHVCA

SEQ ID NO: 47 - protein, 9 aa, synthetic construct
NSFEVAVCA

SEQ ID NO: 48 - protein, 9 aa, synthetic construct
NSFEVHACA

SEQ ID NO: 49 - protein, 9 aa, synthetic construct
NSFEVHVAA

SEQ ID NO: 50 - protein, 995 aa, synthetic construct
VTQSPRNLVAVTGGKVTLQCAQDRNPHNNMYWYRIDTGQGLRLIHYSASEGTTEKGDIPDGYKASRPSQENFSLILESATPSQTSVYFCATKSSLTYEQYFGPGTRLTVLEGGGGSGGGSGPPSGGGGSQQKVQQSPESLIVPEGGMASLNCTSSNSMFDFWWYRQHSGKSPKMLMSISSIKDKNEEGRFTVHLNKASSHTSLHIRDSQPSDSALYWCAASDNDMRFGCGTKLIIKPDGGGGSEVQLVESGGGLVQPGGSLRLSCAASGYSFTGYTMNWVRQAPGCGLEWVALINPYKGVSTYNQKFKDRFTISVDKSKNTAYLQMNSLRAEDTAVYYCARSGYYGDSDWYFDVWGQGTLVTVSSGGGGSGGGGSCPPCGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPG

SEQ ID NO: 51 - protein, 995 aa, synthetic construct
VTQSPRNLVAVTGGKVTLQCAQDRNNHNNMYWYRIDTGHGLRLIHYSASVITTEKGDIPDGYKASRPSSENFSLILESATPSQTSVYFCATSSDSTAEQYFGPGTRLTVLEGGGGSGGGSGPPAGGGGSQQKVEQSPESAVVPEGGMASLNCTSSNSMFDFWWYRQISGKGPKMLMSISSIKDKNEEGRFTVHLNKASSQTSLHIRGSQPSDSALYWCAASDNFMRFGQGTKLIIKPDGGGGSEVQLVESGGGLVQPGGSLRLSCAASGYSFTGYTMNWVRQAPGCGLEWVALINPYKGVSTYNQKFKDRFTISVDKSKNTAYLQMNSLRAEDTAVYYCARSGYYGDSDWYFDVWGQGTLVTVSSGGGGSGGGGSCPPCGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPG

SEQ ID NO: 52 - protein, 1201 aa, synthetic construct
QQMVQQSPESLIVPEGLLLSLACTPSDSLFDFWWYRQHSGKSPKMLMSISSIKDKKEEGRFTVHPNKASSHISLHIRDSQPSDSALYICAASDGDMRFGCGTKLIIKPDGRPDPAVYQLRDSKSSDKSVCLFTDFDSQTNVSQSKDSDVYITDKTVLDMRSMDFKSNSAVAWSNKSDFACANAFNNSIIPEDTFFPSPGGGGSGGGGSGGGGSCGLEAAVTQSPRNLVAVTGGKVTLQCAQDRNPHNNMYWYRQDTGCGLRLIHYSASEGTTEKGDIPDGYKASRPSQENFSLILESATPSQTSVYFCATSSDSTAEQYFGPGTRLTVLETRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 1242 aa, synthetic construct
QQMVQQSPESLIVPEGLLLSLACTPSDSLFDFWWYRQHSGKSPKMLMSISSIKDKKEEGRFTVHPNKASSHISLHIRDSQPSDSALYICAASDGDMRFGCGTKLIIKPDGRPDPAVYQLRDSKSSDKSVCLFTDFDSQTNVSQSKDSDVYITDKTVLDMRSMDFKSNSAVAWSNKSDFACANAFNNSIIPEDTFFPSPGGGGSGGGGSGGGGSCGLEAAVTQSPRNLVAVTGGKVTLQCAQDRNPHNNMYWYRQDTGCGLRLIHYSASEGTTEKGDIPDGYKASRPSQENFSLILESATPSQTSVYFCATSSDSTAEQYFGPGTRLTVLETRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPESKYGPPCPPCPAPEAAGGPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKESKYGPPCPPCPAPEAAGGPGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 54 - protein, 1003 aa, synthetic construct
CGLEAAVTQSPRNLVAVTGGKVTLQCAQDRNPHNNMYWYRQDTGCGLRLIHYSASEGTTEKGDIPDGYKASRPSQENFSLILESATPSQTSVYFCATSSDSTAEQYFGPGTRLTVLEGGGGSGGGSCPPCGGGGSQQMVQQSPESLIVPEGLLLSLACTPSDSLFDFWWYRQHSGKSPKMLMSISSIKDKKEEGRFTVHPNKASSHISLHIRDSQPSDSALYICAASDGDMRFGCGTKLIIKPDGRGGGGSEVQLVESGGGLVQPGGSLRLSCAASGYSFTGYTMNWVRQAPGCGLEWVALINPYKGVSTYNQKFKDRFTISVDKSKNTAYLQMNSLRAEDTAVYYCARSGYYGDSDWYFDVWGQGTLVTVSSGGGGSGGGGSCPPCGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPG

SEQ ID NO: 55 - protein, 1003 aa, synthetic construct
CGLEAAVTQSPRNLVAVTGGKVTLQCAQDRNPHNNMYWYRQDTGCGLRLIHYSASEGTTEKGDIPDGYKASRPSQENFSLILESATPSQTSVYFCATSSDSTAEQYFGPGTRLTVLEGGGGSGGGSCPPCGGGGSQQMVQQSPESLIVPEGLLLSLACTPSDSLFDFWWYRQHSGKSPKMLMSISSIKDKKEEGRFTVHPNKASSHISLHIRDSQPSDSALYICAASDGDMRFGCGTKLIIKPDGRGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPGGGGGSEVQLVESGGGLVQPGGSLRLSCAASGYSFTGYTMNWVRQAPGCGLEWVALINPYKGVSTYNQKFKDRFTISVDKSKNTAYLQMNSLRAEDTAVYYCARSGYYGDSDWYFDVWGQGTLVTVSSGGGGSGGGGSCPPCGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 56 - DNA, 2985 bp, synthetic construct
  source 1..2985: mol_type = other DNA
gtcacacaaagccccagaaacctggttgccgtgactggaggcaaggtgactctgcaatgcgcccaggatcgcaaccctcataacaacatgtattggtatagaatcgacacaggtcagggcctcaggctgattcactatagcgcctccgagggcaccacagagaagggcgacattcctgatggttacaaggctagccgcccgtcacaggaaaactttagccttatcctggagtctgcaaccccgagccaaacttccgtctatttctgcgccactaaatcctcactgacctacgagcaatacttcggtcccggcacaaggttgacagtactggaggggggtggtggaagcgggggcggctccggccccccctctggcggtggaggctctcaacaaaaggtccagcagagcccagagagtctgattgtacccgaagggggaatggcctctctgaattgcacctccagtaactcaatgtttgacttttggtggtatagacagcactctggcaaatcccctaaaatgctgatgtccatcagcagcatcaaggataagaacgaggagggccggtttactgtgcacctcaacaaggccagttcccacacaagcttgcacatcagagactctcagccgtccgactcagccttgtattggtgcgcggcaagcgacaacgatatgaggtttggctgcgggacaaaacttatcatcaagcctgatggcggcggcgggtcagaagtccagctggtggaaagtgggggggggctcgtacagccagggggtagcctgagactgtcatgcgcagcttcaggttacagcttcacaggttataccatgaattgggttcgacaggctcccgggtgtggactggagtgggttgcactgatcaatccgtacaaaggtgtcagcacatataatcagaaatttaaagaccgcttcaccatcagcgtcgataaaagtaagaacactgcatatctgcagatgaattccttgcgagcagaggataccgcagtgtattactgcgcacggtccgggtattacggggatagtgattggtacttcgatgtctggggacagggaacccttgtgacagtgtcaagtggcggtggaggatccggaggcgggggaagctgcccgccatgtggtgggggggggtctgcaatccagatgactcagtccccttcaagcctgagcgcatctgtcggggacagggtaactattacatgccgggcttcccaggacataagaaattacctgaactggtatcagcagaaaccaggcaaggctcctaagctgctcatctactatacctcacgcttggagagcggggttcctagtcggttttccggtagtggctccgggaccgactatacccttactatctcttccctccaacccgaagatttcgccacatactattgtcagcaaggaaacactctgccttggaccttcggctgcggcactaaggtggaaattaaaggtggcgggggctccgacaaaacccatacctgccctccttgcccagcgcccgaagccgctgggggcccttccgtgttcctgttccctcccaaacccaaggatactttgatgataagtcgcacacccgaagtgacatgcgttgtggttgacgtaagccacgaagaccccgaagtcaaattcaactggtacgtggacggggtagaggtgcacaacgctaaaactaaaccttgcgaagagcagtacggatccacatacaggtgcgtgtctgtcctgacagtgctgcaccaggactggctcaatgggaaggaatacaagtgtaaagtatctaataaggccttgccagcaccgatcgaaaagactatttcaaaggccaagggtcagcctcgcgaaccacaggtgtatactctgccaccaagtcgggaggaaatgaccaagaaccaggtctctcttacctgcctcgtcaagggtttttatccgtctgatatcgccgttgagtgggaaagcaatgggcagcctgagaataattataaaacgactcctccagttcttgattctgacggctctttctttctgacgagtaagctgaccgtggataagagccgctggcagcaaggcaatgtgttcagctgtagtgtgatgcatgaggctctgcataaccactatacgcagaagtctcttagcctctctcctgggggtggaggaagcggtggaggtggctccggaggcggtggcagcggaggaggcggatcaggcggaggagggtccgggggcggcggctctgataagactcatacctgccccccctgtcctgcgcccgaagccgcaggaggcccttccgtgttcctgtttccccctaagccaaaggacactctgatgatttcaagaacacccgaggtgacatgcgtggtggttgacgtttcccatgaagatccagaagtaaagtttaactggtacgtcgatggcgtggaggttcataatgcgaagactaagccatgcgaggaacaatatggtagcacctatagatgtgtgtcagtactcactgtgctgcatcaggattggctcaacggcaaagagtacaagtgtaaagtaagtaataaggctctgcctgcacctattgagaaaacaatcagtaaagcaaagggtcagcctagagagccacaagtttatactctgccaccatcaagggaagagatgacaaaaaatcaggtgtccctctattgtctggttaagggcttttaccctagtgacatcgctgttgagtgggaatcaaatggccaacccgagaacaattacaagaccacacccccagtgctcgatagtgacggcagctttttcttgtatagcaaattgactgtggacaagtctcggtggcagcaggggaacgtttttagctgctccgttatgcacgaggccctgcataaccactatacccagaagtccctgtctctgtctcccggcaaggaaggcaggggaagcctgctgacatgcggcgatgtagaagagaatccagga

SEQ ID NO: 57 - DNA, 2985 bp, synthetic construct
  source 1..2985: mol_type = other DNA
gtgacccaatcacctagaaatttggtcgcggtcaccggaggtaaggtgacacttcaatgtgcccaggataggaacaaccacaataatatgtactggtacaggattgatacggggcacggcctgagactgattcactattccgcctctgtgatcacaacagagaaaggcgatatcccagacggatacaaggctagcaggcctagctccgaaaacttctcactcatcctggagtccgccaccccctctcagacgtcagtttacttctgtgctactagttcagacagcactgcagagcagtatttcggccctggtacccgcttgactgtactggagggaggaggcggatccggcggcggaagtggtcctcccgctggaggcggaggttcacagcagaaagtggaacagtcccctgaaagcgccgttgtgccggaaggcggaatggcgtctctgaattgtaccagttctaattccatgttcgatttctggtggtaccgccaaatctcaggaaagggtcccaagatgctgatgtctatcagcagcataaaggacaagaatgaggagggcagatttaccgtgcacctcaataaagcgtctagtcagacctccctccacattcgggggagccaaccgtccgatagcgctttgtattggtgcgcagcctctgacaattttatgcgatttgggcagggcacaaagctcataataaagcccgatgggggcgggggctctgaggtgcagctggttgagtctggtgggggacttgtgcaaccaggcgggagtctcaggctcagctgcgctgcctcaggatactcctttacaggatacacaatgaattgggtgagacaagcacccggctgcgggttggagtgggtggctctgatcaacccgtataaaggagtctctacgtataaccaaaagtttaaagacagatttaccataagtgttgataagagcaagaatacagcttatctgcaaatgaactctctgcgggccgaggacacagctgtttattattgcgctagaagcggctattacggcgactctgactggtattttgacgtttggggccaggggacactggtgaccgttagctccggcggaggcggttctggcgggggaggatcttgccccccatgtgggggaggcgggtcagccatacagatgacacagtccccttcctccctgtcagcctctgtaggcgatagggtgactattacctgtagggcttcacaggatataagaaattatctcaattggtaccaacaaaaacctggcaaagcccccaagctccttatctactacactagtagactggaaagcggggtgccttcaagattcagcggctccggatctggcactgattacacacttacaatttcttcacttcagcctgaggatttcgctacttactactgtcaacaaggcaatacgctgccgtggacttttggttgcggcacaaaagttgagatcaagggaggcggaggctctgacaagacccatacgtgcccaccttgccccgcaccagaggcagcgggggggccttcagtgtttcttttcccaccgaagcctaaggacactctcatgatctcccgcacccccgaggtgacgtgcgtggtagtcgacgtctctcacgaggatccagaagttaaatttaattggtatgtcgacggcgtcgaggtgcacaatgctaagaccaagccttgtgaggagcagtacggcagtacttataggtgtgtgagcgttcttactgttttgcaccaggactggctgaatggcaaggagtacaagtgtaaggtttccaataaggccttgcctgcaccaatcgaaaaaaccatctctaaagcaaaaggacaaccgcgagagcctcaggtctatactttgccaccatcccgcgaagagatgacaaaaaatcaggtgtctctgacctgtctggttaaggggttctacccttctgacatcgcagtggagtgggagagtaatggccaacccgagaacaattataaaacaaccccacctgttctggacagtgatggatctttctttctgacatccaaactcaccgtcgacaagagcaggtggcagcagggaaatgtgttttcctgttcagtgatgcatgaggccctgcacaaccactacacgcagaaaagcctcagtctttcacctggtgggggcggctctggcggaggtggcagcggcgggggaggaagcggtggcggggggagcggcggaggcgggagtggcggaggaggaagtgataaaacccatacttgccccccctgcccagcccccgaggccgccggaggaccctctgtgttcctgttccctcccaaacctaaggacaccttgatgatttccagaacacctgaggtcacctgtgtggtcgtagacgtgagccatgaggacccagaggtgaagtttaattggtatgttgacggggttgaagttcacaacgctaaaaccaaaccatgcgaagagcagtacggaagcacctaccggtgtgttagtgtcctgactgtgcttcaccaggattggctgaacggcaaggaatataaatgtaaggtctctaataaagcattgccggctcctatcgagaaaactataagcaaagctaagggccaaccaagggagccgcaggtctacaccctgcctcccagcagggaagagatgacaaagaatcaagtcagcctctattgcctggttaagggattctacccatctgacattgcagttgaatgggagagcaacgggcaaccagaaaacaactacaaaactaccccgcctgtgcttgacagcgacgggagctttttcctgtactcaaaactcacagtcgataaatccagatggcagcaggggaacgtattctcttgctctgtgatgcatgaagcccttcacaaccactacactcaaaaatctctgtccctgagccccggcaaggagggccgcgggtctctgctgacttgtggagatgtggaggaaaaccccggg

SEQ ID NO: 58 - DNA, 3603 bp, synthetic construct
  source 1..3603: mol_type = other DNA
cagcagatggtgcagcaaagtccagagagcctgattgttccagaaggtctgcttctgagtctcgcctgtaccccttccgattccctgtttgatttctggtggtaccggcaacactctgggaagtcacccaagatgctgatgtccatctccagcatcaaggataagaaagaggagggccggtttacagtgcatccgaacaaagccagctctcacatttcccttcacattcgcgactcccagcccagtgactctgcactctacatctgcgccgccagcgacggtgatatgcgcttcgggtgcgggaccaaattgatcattaaacctgacggaagaccagacccggccgtttatcagctgagggatagcaagagcagcgacaaaagcgtctgtctgttcaccgattttgactctcagacaaacgtgtcacagagcaaagatagtgatgtgtatattacagacaaaaccgtccttgacatgcgctcaatggacttcaaatccaatagcgcagtggcatggtcaaataaatccgatttcgcttgtgccaatgctttcaacaattccattattccagaagatactttttttccctcccctggaggaggcgggtcaggcggcggggggagcggtgggggtgggagctgcggactggaggccgcagttacgcaatccccgaggaatctggttgccgttaccggcgggaaagtgaccctccaatgtgcccaggatagaaacccccataataacatgtattggtatcgccaggacaccgggtgtggcctccgacttatccactatagcgcctcagagggcacgacagaaaaaggggatatcccggacggctataaagcaagcagaccaagccaggaaaatttctcactgatcctcgaatcagctacgccaagtcagacctcagtctatttctgtgctaccagttccgattcaaccgccgagcagtatttcggaccaggcaccagactcactgtgctggagacacggctgacagtactcgaggacctcaacaaggtttttccacccgaggtggctgtattcgaaccctctgaagccgagataagccacacccaaaaagctaccttggtgtgcctggcaactggattctttccggaccatgtggagctttcttggtgggttaatggcaaagaggtgcattcaggcgtatctaccgaccctcagcccctgaaagaacagccagccctcaatgattcccgatactgcttgtctagtagactcagggtctccgctacgttctggcagaaccctcgaaatcactttaggtgccaggttcagttctatgggctttccgaaaatgatgagtggacccaagacagagcaaagcctgtcacgcaaattgtatcagctgaggcatggggcagggcggacggcgggggaagtgaagtccaattggtggagagtggcggcggattggtccagcctgggggatctctgcgcctctcctgcgctgcttctgggtattccttcaccggctataccatgaattgggtcagacaggctcccgggaaggggctggaatgggtagccctcattaatccatacaagggagtgtccacctacaaccagaagttcaaggatagattcacaatctccgtggacaagtcaaaaaatacagcgtaccttcagatgaattcccttcgagccgaagacaccgctgtgtactactgcgccagatctggctactacggagactccgattggtattttgacgtttggggtcagggaactctcgtgaccgtgtcctccggagggggagggtcaggtggcggagggtccggaggtggaggtagtgccattcaaatgacccagtccccttcatctcttagcgcaagtgtgggtgaccgagtaactattacttgtagagcctcccaggacatccgaaattatctcaattggtaccagcagaagccaggtaaggctcccaaactgctcatttactacacctctagactggagagcggggttccaagccggttttctggcagtgggtcaggtactgactatacactgaccatcagcagccttcagccagaagacttcgcaacatactactgtcagcaaggcaacacacttccctggacctttggccagggcacaaaagtggagatcaaaggaggggggggcagcgataagactcacacctgccccccttgccctgcccccgaagccgctggaggcccatccgtcttcctcttcccaccgaagcctaaggatacgttgatgatatccaggaccccagaggttacatgtgtggttgtggacgtctcccacgaggacccggaagttaagttcaattggtatgtggacggggtggaggtgcataatgcaaaaaccaagccttgcgaggagcaatacgggagcacctaccgctgtgtgtccgtgctgacggtgttgcatcaagactggctcaatgggaaggagtataaatgtaaggtatctaataaagctctgcccgcccctattgagaaaacgatctccaaagctaaggggcagccacgggagcctcaggtgtacacccttcctccttctagagaggagatgaccaaaaatcaggtctccctgacctgtttggttaaagggttctacccctcagacatcgccgtggagtgggaatcaaacggtcagcctgagaataattataagacaactcccccagtgctggacagcgatgggtcattctttctgacaagtaaactgaccgtggataaaagtaggtggcagcaaggcaatgtgttctcttgttctgtgatgcacgaggccctgcacaatcactacacacagaaatccttgagcttgtctccgggaggcggcggtagtggaggtggcgggagtgggggcggagggagtggtggtggggggagcggcgggggtggatctgggggcggcggatctgataagacacatacctgcccaccgtgcccagccccggaagcagctggcggcccaagcgtctttctgttccccccaaaacccaaagacacgctcatgattagcagaaccccagaagtgacatgcgtggtggtagacgtgagccacgaagacccagaggtgaagtttaactggtacgtagatggcgttgaggtgcacaacgccaagactaagccctgcgaggagcagtatggcagcacctaccgctgtgtgagcgtgctgacagtcctgcatcaagattggttgaacggaaaagaatataagtgtaaagtctctaacaaggctttgccagcccctatcgaaaagacgatcagtaaggccaaaggccaacccagagagcctcaggtatacactctgcccccttcacgggaggagatgactaaaaatcaagttagcctgtattgtctggttaaagggttctatccatctgacattgctgtggaatgggaatccaatggccagccagagaataattataagacaacaccccccgtgttggattcagacggctctttctttctctatagcaagctcacggtggacaaaagtcggtggcagcagggtaatgtgtttagctgctctgtgatgcacgaagcacttcacaatcactatactcagaagtctctgagtctgtccccggggaag

SEQ ID NO: 59 - DNA, 3726 bp, synthetic construct
  source 1..3726: mol_type = other DNA
caacagatggtccagcagtctcctgagtctctcatagttccagaaggtctcctgctgtctctcgcatgcactccatccgattccctgttcgatttctggtggtacaggcagcacagcggaaagagtcctaaaatgctcatgagcatcagctcaatcaaggataagaaggaagaaggccgctttacagtgcatcccaataaagcgtcctcacatattagcctgcacataagagactctcagccaagcgatagcgctttgtatatctgtgccgcttctgacggagatatgcggtttggatgtgggacgaaattgatcatcaaacccgatggacgccccgacccagctgtataccagctgcgcgacagcaagagcagcgacaaatccgtctgcctcttcaccgattttgacagccaaaccaatgtctcacagagcaaagactctgacgtgtatatcacagacaaaaccgtattggatatgcgcagcatggacttcaaaagcaactctgccgtggcgtggagcaataagtcagatttcgcctgtgcaaacgcctttaataactccatcattccggaagacacattctttccatcacctggagggggcggttctggcggtggcggatctgggggaggcggatcctgcggtcttgaggccgctgtaacgcaaagccctcggaatctcgtagctgtgaccgggggaaaagttactcttcaatgtgctcaggatagaaatccccacaacaatatgtattggtaccgacaagacaccggatgtggactgcgcttgatccactacagtgcctccgagggcaccacagagaaaggcgacatacctgacggatataaagcctctcgcccatcacaggaaaatttctccctcatactggagtccgccaccccatctcagacttcagtttacttttgtgccacctctagtgacagtacagcagaacagtacttcggacccggcaccagactgaccgtactggaaacgcggctgacagtcttggaggaccttaataaggttttccctcctgaggtggctgtttttgagccctctgaggccgagatttctcatacgcagaaagcaaccctcgtgtgcctggcaacggggttcttccctgaccacgtagaattgagttggtgggtgaatgggaaagaagtacactccggtgtcagcactgatccacagccgctgaaagaacagccagcccttaacgacagtagatactgcctgagctcccgcctccgggtgtctgcgaccttctggcaaaatcctcgcaaccacttcaggtgccaggtacaattctacggacttagtgaaaatgatgagtggacgcaggaccgcgccaagccggttacccagatcgtatccgccgaggcttggggccgcgccgatggaggaggtggatctgataaaacccacacctgtccaccctgtcccgctccagaggcagctgggggaccctccgtgtttctgttccctcctaaaccgaaggacacactgatgatctctcgcaccccagaagtcacttgcgtagtcgtggatgtcagccatgaggatcccgaagtgaaatttaattggtatgtggatggggttgaggtgcacaatgctaaaacaaagccatgtgaagaacaatacggttctacttatcggtgcgtcagcgtccttacggtgctgcaccaagactggctgaacggaaaggaatataagtgcaaggtgagcaacaaagctctgccagcccccatcgaaaagacaattagtaaagcaaagggtcagccgcgcgaaccacaggtatatacgcttcctccatccagagaggaaatgacaaagaaccaggtgtctcttacctgcctcgtaaagggtttctaccccagcgacatcgcagtggaatgggagagcaacggtcaacccgagaacaactacaagacaactcctccagtgcttgacagcgatggaagctttttcttgaccagtaaacttactgtcgataaatcaaggtggcagcagggcaatgtattttcttgttccgttatgcatgaagccctgcataaccactatacacagaagtctctctctctgagtcctgaatctaaatacggcccaccttgccctccttgccccgcacccgaggctgcgggtggccctggtgggggcggctctggcgggggcgggagcggaggagggggctccggcggaggtgggtctgggggtgggggctcaggaggcggcgggtccgataagacacatacctgcccaccttgcccagctcctgaggcggcaggagggccaagtgtgttcctgtttcctcccaaacccaaggacacccttatgatttcacgaacacccgaggtgacttgtgtggttgttgacgtgtctcacgaagatcctgaggtaaaattcaattggtacgttgacggcgtcgaggtgcacaacgcaaagaccaagccttgcgaagagcagtatggcagtacctataggtgcgtgtccgtcttgacagttctccaccaggactggttgaatggcaaggaatataagtgcaaggtatcaaacaaggcccttcctgctcccatcgaaaagaccatcagcaaggctaaggggcagccccgagagcctcaagtgtacaccttgccacccagcagggaggaaatgactaagaatcaggttagcttgtactgcctcgtgaaaggcttctacccttccgatatcgctgtggagtgggagagcaatgggcagccggaaaataactacaagactacccccccagtgcttgacagcgacgggagcttctttctctattctaaactgacggtggataagagcaggtggcaacagggaaacgtcttttcctgctcagtgatgcacgaggcactgcacaatcattacacccaaaagagcctgtccctgtctcccggcaaagagagcaaatatggccctccgtgtccaccgtgtccggcgccggaagccgccggagggcccgggggaggaggcagcgaagtgcagcttgtggagtccggcggcggcctggtgcagcctggtggcagcctgcggctgagttgcgccgccagcggctactccttcacgggttacacaatgaactgggtcagacaggcacctggtaaaggtcttgagtgggtagcactgattaacccctataagggagtgagtacttataatcaaaaattcaaggatcgctttaccatttccgtagacaaatcaaagaacaccgcttaccttcagatgaactccctccgggctgaggacaccgcagtgtattattgtgcgcgctcaggctactacggtgactctgattggtacttcgacgtgtggggccaaggcactctggttactgttagcagcggaggcggagggtcaggtgggggcggctcagggggagggggcagcgccattcagatgacccagtccccgtcctccctctctgcctccgtgggtgatagagtgaccataacatgtagagcgtctcaggacatcagaaattatttgaactggtaccagcagaaacccggcaaggcccccaagcttcttatatattatacatctagactggaatctggagtcccaagcaggtttagcggatctggatccggcacggattatacgctcaccatatcctcactccagcctgaggacttcgctacatattactgccagcagggaaacacacttccctggacttttggtcaggggacgaaggtagagatcaaa

SEQ ID NO: 60 - DNA, 3009 bp, synthetic construct
  source 1..3009: mol_type = other DNA
tgcggtcttgaggcagccgtcacccagtccccaaggaatctggttgctgtaaccgggggaaaagttacgctgcagtgcgcacaggatcgaaacccccacaacaacatgtattggtataggcaggatactggatgcggacttagactgattcactacagtgctagcgagggtacaacagagaagggcgacatccccgacggatacaaggcctccagacctagccaggagaacttcagtctcattttggaaagcgccacgccaagtcagacgagcgtctacttttgtgccacctccagcgacagtaccgccgagcagtactttgggcccgggacccgactcaccgtgctggagggcggcggagggagcggcggtgggagttgccccccctgtggcggggggggatcccagcaaatggtgcagcaatctcctgaaagcctgatcgtccccgaaggtctgctgttgagcctggcgtgcactccctccgacagcctgttcgatttttggtggtaccgccagcactcaggcaaatccccaaaaatgctgatgagcatttcaagtatcaaggataaaaaagaagaaggacggtttactgtgcaccctaataaagctagcagccatatcagcctgcatatcagggactcccagccctcagactccgcgctgtatatttgtgcagcctccgacggggacatgcgattcggatgcggaacaaagttgataataaagccggatgggcgcggaggcggcggctcagaagttcagctggtagaaagtggcggcgggcttgtgcagcccggagggagcttgcggctttcttgcgccgcatcaggttattcctttacgggttacacaatgaactgggtgaggcaggctccaggatgcggccttgagtgggttgctctgattaatccctacaagggcgtcagcacctataatcagaaatttaaggatcggttcactataagtgtggataaaagcaagaatacggcatacttgcagatgaattccctcagggcagaagatacagccgtctattattgcgctaggtcaggctactatggcgattccgactggtacttcgatgtgtggggacagggcacactcgttacagtctcctcaggagggggcgggtctggcggcggtgggtcatgcccaccctgtggaggcggaggttctgccattcaaatgacccagagtccttcctcactttcagccagtgtgggagaccgggtcacgattacctgcagggcttcccaggacattagaaactacttgaactggtaccagcagaagcccgggaaagcacccaagcttctgatctactataccagtagactcgaaagcggggtgcctagtaggttctctggcagcggtagcgggaccgactacaccctgacaataagctctctccaacctgaggacttcgccacttactattgccagcagggaaacaccctcccatggacttttggttgtggaacgaaggtcgaaattaaagggggaggaggcagcgacaagacccacacatgcccgccgtgtccagccccagaggcggccggtggcccaagtgtctttctcttccccccaaagccgaaggacacgttgatgatcagtagaacacccgaggtgacctgtgtggtggtcgatgtttcccatgaagatcccgaagtcaaattcaattggtacgtggacggagtcgaagttcacaacgctaagactaaaccatgtgaagagcaatatggatccacctacagatgcgtttctgtcctgactgttctccatcaggactggcttaacggaaaggagtacaagtgtaaagttagtaacaaggcccttcccgcccccattgagaaaacaattagcaaggccaaggggcaaccccgcgaaccacaggtttacacacttccgccatccagggaagaaatgactaagaatcaggtgtccttgacctgcttggtgaagggattttatccgtctgacatcgcagtcgagtgggagtccaacgggcagcctgagaataattacaagaccactccccccgttctggactccgacgggtcctttttcctgacctcaaaactgacagtggataagagcagatggcagcaagggaacgtttttagctgttcagttatgcacgaggccctccacaatcattacacacagaaatcactgtccctgtcccctggcggaggaggcagcggtgggggcgggagcggtggggggggatccggcggaggcggaagcggtggtggtgggagtggaggcgggggctccgataaaacacacacttgccctccatgtcctgctcccgaggcggcgggaggtccctctgtgtttctgtttcccccaaagccaaaagacacactgatgatctccagaactcccgaagtaacatgcgtcgtcgtggatgtctctcacgaagacccagaagtcaagttcaattggtacgtcgacggcgtggaggtgcacaatgcaaagacgaagccttgcgaagaacagtacggcagtacttaccgctgtgtgtcagtgctcacagttctgcaccaggattggttgaacgggaaggagtacaagtgtaaggtctccaataaggcccttccagcaccaattgaaaagacaatctccaaagccaaaggccagccgcgcgagccacaggtgtacactctcccaccatctagggaggagatgaccaaaaaccaagtgtccctgtattgtcttgtgaagggcttctacccttcagacatagccgtagagtgggagagtaacggccaacccgagaacaattacaaaacaacacctccggtcctcgatagtgacggttctttcttcctgtattctaaactcactgtggacaagtccaggtggcagcaagggaatgtattttcatgttctgtgatgcatgaggctctccacaatcactacacccagaaaagtctctcactgtccccaggaaaggagggtcgcggatcccttctgacttgcggagatgtcgaagaaaacccaggg

SEQ ID NO: 61 - DNA, 3009 bp, synthetic construct
  source 1..3009: mol_type = other DNA
tgcggactggaggccgccgtgactcaaagccccagaaatttggtggccgtcacaggcggaaaagtcaccctccaatgcgcccaagatcgcaaccctcacaataatatgtattggtataggcaagataccggatgtggcctgcggctgatccattattctgctagcgaggggaccaccgagaaaggtgacataccagatggctataaggcatcacgaccgtctcaggagaatttttcactgatcctggaatccgccactcctagccaaacaagtgtttacttctgcgcaacgtctagcgacagtacagccgagcagtactttggccccggtactcggctcaccgtattggaaggcggtggtggcagtggtggagggtcctgtcccccgtgtggcggcgggggctcccagcagatggtccaacaatcaccagagtctctgattgtccccgaaggcctgctcctctccttggcctgtacaccatcagacagccttttcgacttttggtggtatcgacagcattctggcaaatcacctaaaatgctgatgtccatcagcagcatcaaagataagaaagaagagggccgattcaccgttcatcctaataaggccagctcccatatttctctgcatatacgggattctcagccaagtgactctgccctgtatatctgtgcagcatctgatggagacatgaggttcggttgtggcactaagttgataattaaaccagacggccgcgggggcggaggcagcgataagactcatacatgccctccgtgtccagcacccgaagcggctggagggccatcagtgttcctcttccctcctaagccaaaagacaccctgatgatcagtcgaacgcccgaggtgacctgcgtcgtagtagatgtctcacacgaagaccccgaggttaaatttaactggtacgtcgacggcgtcgaagtccacaatgcaaaaaccaagccctgtgaggaacagtatgggagcacttatcggtgtgtgagcgtactgacagtattgcaccaagactggctgaacggcaaagaatacaagtgtaaggtaagcaacaaggctctgccagcgcctattgagaagactatttccaaggccaagggtcagcccagagaaccacaagtgtatacacttccacctagcagagaggaaatgaccaagaaccaggtgtccttgacctgtctggtcaaagggttctatccaagcgatatcgcagtggaatgggagtcaaacggccaacccgagaacaactataagaccacacctccagtgcttgactctgatggaagtttttttctgacctcaaaactcaccgtggataagagtcggtggcagcaaggcaacgtgttcagctgttccgtgatgcacgaagctctgcataatcactacacgcagaaaagtctctccctttctcctgggggaggaggcagtggagggggaggatccggaggaggcggtagtggcggcggggggtcaggtggtgggggatctggtggaggtggaagcgacaagactcacacttgtccaccttgtccggcacctgaggcggcgggaggcccaagcgtgttcctgtttcctccaaaacccaaggataccttgatgatcagcagaactccagaggtgacatgtgtagttgtggacgtttcacacgaggatcctgaagtgaagttcaactggtatgtggatggggttgaagttcacaatgcaaaaactaagccttgtgaagaacagtacggatcaacctatagatgtgtgtccgtcttgacagtgcttcatcaagactggctcaatggcaaggaatataaatgcaaagtctcaaacaaagccttgccggccccaatcgaaaagactatctctaaggccaagggccagccccgggagcctcaggtgtacacgctcccaccctcaagagaagaaatgactaagaatcaggtcagcttgtactgtttggtcaagggattttatccatctgatattgccgttgagtgggaatcaaacggacagcctgagaataactataagacaactcctcccgttctggattcagatgggtcctttttcttgtactctaagctcaccgtggataagtctagatggcagcaaggaaacgtgtttagctgctcagtgatgcatgaagcccttcacaaccactacacccagaagtcactgagcctcagccccggcaaggagggaaggggtagcctgttgacatgcggcgatgtggaggaaaatcctggcggaggtgggggttctgaggtacagctggtggagagcggcggtggcttggttcagcccggaggctcactgaggcttagctgtgccgccagcgggtactctttcactggctacacaatgaactgggtgcgacaggcacccggttgcggattggaatgggtggccttgattaacccctacaagggggtgagcacatataatcaaaagtttaaggacagatttaccataagtgtcgacaagtcaaaaaacacagcctatctccagatgaacagcctgcgggccgaagacactgcagtatattactgtgcacggtccggctactatggcgatagtgattggtactttgacgtatggggccaaggtacactggtgaccgtatcttcaggaggtggcggttcagggggtggggggagttgcccaccttgtggcggaggaggcagtgccattcagatgacccagtcacctagttccctgtctgcaagtgtcggcgacagagttacaataacatgcagggcctctcaggacattagaaactaccttaattggtatcagcagaaacctggaaaagcccctaaactgctcatctactatactagcagactggagtccggcgtgccttcccggttctcagggtccgggtctggcactgactacacgctcactatcagctccctgcagcctgaagactttgctacttattattgccagcaaggcaataccctgccctggactttcgggtgcggtacgaaggttgagatcaag

SEQ ID NO: 62 - protein, 1192 aa, Homo sapiens
QQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGSGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPGGGGSGGGGSGGGGSEAAVTQSPRNKVAVTGGKVTLQCAQDMNNHNNMYWYRQDTGHGLRLIHYSASEGTTEKGDIPDGYKASRPSQENFSLILELATPSQTSVYFCATKSSLTYEQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 1233 aa, Homo sapiens
VKQNSPSLSVQEGRISILNCDYTNSMFDYFLWYKKYPAEGPTFLISISSIKDKNEDGRFTVFLNKSAKHLSLHIVPSQPGDSAVYFCAASDNDMRFGAGTRLTVKPIQNPEPAVYQLKDPRSQDSTLCLFTDFDSQINVPKTMESGTFITDKTVLDMKAMDSKSNGAIAWSNQTSFTCQDIFKETNATYPSSDVPCDATLTGGGGSGGGGSGGGGSVTQTPKFQVLKTGQSMTLQCAQDMNHNSMYWYRQDPGMGLRLIYYSASEGTTDKGEVPNGYNVSRLNKREFSLRLESAAPSQTSVYFCATKSSLTYEQYFGPGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSAGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPESKYGPPCPPCPAPEAAGGPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKESKYGPPCPPCPAPEAAGGPGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 64 - protein, 999 aa, Homo sapiens
EAAVTQSPRNKVAVTGGKVTLQCAQDMNNHNNMYWYRQDTGCGLRLIHYSASEGTTEKGDIPDGYKASRPSQENFSLILELATPSQTSVYFCATKSSLTYEQYFGPGTRLTVLEGGGGSGGGGSCPPCGGGGSQQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGCGTKLIIKPDGGGGSEVQLVESGGGLVQPGGSLRLSCAASGYSFTGYTMNWVRQAPGCGLEWVALINPYKGVSTYNQKFKDRFTISVDKSKNTAYLQMNSLRAEDTAVYYCARSGYYGDSDWYFDVWGQGTLVTVSSGGGGSGGGGSCPPCGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPG

SEQ ID NO: 65 - protein, 978 aa, Homo sapiens
EAAVTQSPRNKVAVTGGKVTLQCAQDMNNHNNMYWYRQDTGCGLRLIHYSASEGTTEKGDIPDGYKASRPSQENFSLILELATPSQTSVYFCATKSSLTYEQYFGPGTRLTVLEGGGGSGGGGSCPPCGGGGSQQKVQQSPESLIVPEGGMASLNCTSSNSMFDFWWYRQHSGKSPKMLMSISSIKDKNEEGRFTVHLNKASLHTSLHIRDSQPSDSALYLCAASDNDMRFGCGTKLIIKPDGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 66 - DNA, 3576 bp, Homo sapiens
  source 1..3576: mol_type = genomic DNA
cagcagaaagttcagcagagtcctgaatcacttattgtcccagagggcggaatggcaagtcttaattgtacctcatctaactcaatgtttgacttctggtggtataggcagcactctggcaaatcaccgaaaatgcttatgtctatatcatccattaaggacaaaaatgaagaagggcgcttcactgtgcatctcaataaagcatctctccacacctcactgcatattcgggatagtcaaccatcagattcagcattgtacctctgcgctgcatcagacaacgacatgcgattcggtagcggtaccaaactgatcatcaaaccggacatccaaaatccagatcctgccgtctatcagctccgggacagcaagtccagcgataaatccgtctgtctttttactgatttcgattctcagacaaatgtatctcaatccaaggactccgatgtgtacatcaccgacaagacagtactggacatgcggtctatggactttaaatcaaattccgccgtggcttggtctaataagtctgacttcgcctgtgccaatgcttttaacaactctattattccagaggacactttctttcccagccccggcgggggtgggtccgggggaggaggttcagggggcgggggcagcgaggccgctgtgacacagagtcctcggaataaggtggctgtgaccggtggcaaggtaacgctccaatgtgcacaagacatgaacaatcataacaacatgtattggtaccggcaagatacgggccacggacttcggctgatccattattccgcgtctgaggggacaacagaaaagggagacattcctgacggctacaaggctagtcggccttcacaggaaaacttctcactgatcctcgaactcgctacaccatcccagacttctgtgtacttttgtgcaaccaaaagctccctgacctatgagcaatactttggacctggcactaggctcacagtgctcgaagatctgaacaaggttttcccgcccgaggtcgccgtgttcgaaccaagtgaagcagaaatttctcacactcagaaagccaccttggtgtgtctggctaccggcttcttcccggatcacgtcgagctttcatggtgggttaatgggaaggaggtccattctggtgtgtctaccgaccctcagcccctgaaggaacagccagccctgaatgatagtcgctactgcctgagttcccgccttagagtgtccgccactttctggcaaaacccacggaatcatttccgctgccaggtccagttttatgggctctcagaaaacgatgagtggactcaggaccgggccaagcctgttacgcagattgtttctgcagaagcttggggcagggctgatggcggcggaagcgaggtgcagctcgttgagagcggcggggggctggtgcagcctggcggctccctgaggctgagttgcgcagcgtccgggtactcttttacagggtacactatgaactgggttaggcaggcccctgggaagggcctcgaatgggttgccctgatcaatccgtacaagggtgtctcaacctataatcagaagttcaaagatcgctttaccatatccgtggacaagtcaaagaacacagcttacctgcagatgaatagcctgagggctgaggacacagccgtgtactactgcgcccgatccggatactatggggacagtgactggtatttcgatgtttggggccaagggacactggttaccgtatcaagcggagggggaggcagcggcggggggggcagcggcggcggcggctcagccattcaaatgacacagtctccttcttccctgtcagcgtctgtaggagatagagtgacgattacatgccgggccagtcaagatattagaaattacttgaactggtaccagcagaaaccgggcaaggccccaaaactgttgatttactataccagtcgcctggagtcaggtgtgccctcacgcttttctgggagcggttccgggaccgactacacacttactatctccagtctgcagcccgaggactttgcaacttattattgtcagcagggtaataccctgccttggacctttggtcaaggcacaaaagttgaaatcaagggggggggcggttccgacaagactcacacttgtcccccctgccctgcccctgaggctgccggaggcccaagcgtattcctcttcccacctaagccgaaggataccctgatgatttctcggactcctgaagtcacctgtgtcgtcgttgacgtgtcccacgaggaccctgaagtgaaattcaattggtacgtggatggtgttgaggtgcacaacgcaaaaacgaagccctgcgaggaacagtacgggtccacctaccgatgtgtgtctgttctgaccgtcctgcatcaggactggctgaatggcaaggagtacaagtgcaaggtctccaataaagcccttccagcgcccattgaaaaaacaattagtaaggccaagggccagcccagagagccacaggtctacaccctgccgccctcacgggaggagatgactaagaatcaagtcagtctgacttgtctggtgaagggattctacccctccgatatcgctgttgagtgggaatctaatgggcagcctgagaacaactacaaaacaacaccccccgtgctggattcagatggctctttttttctgacaagcaagttgactgtagacaaaagcaggtggcagcagggtaacgtcttttcatgctctgtcatgcacgaggcactgcacaaccactacacccaaaagtccttgtctctgtctcctggcggaggtggcagtggtggcggtggctctggaggagggggatctgggggcggcggttcaggaggcggcggcagcgggggaggtggcagtgacaagacccacacctgtcctccctgccctgcccctgaggcagctggaggcccctcagtgttcttgtttccacccaaacccaaagacactcttatgatcagcaggacaccagaagtgacttgtgtggtggtagatgtctcccatgaggaccccgaggtgaagtttaattggtatgtggacggagtggaagtgcataatgcaaagacgaaaccttgtgaagaacagtacggatcaacataccgctgtgtttccgttctcaccgtcctccatcaggattggcttaacgggaaagagtacaaatgtaaagtttcaaataaggccttgcctgcccccattgaaaaaacaatttccaaggctaagggacagccgagagaacctcaagtgtacacattgcctccttcccgggaagagatgacaaagaaccaggtgagcttgtactgcctggtgaagggattctacccctccgacatcgctgtagagtgggagtccaatgggcaacctgagaacaattataagacgacccccccggtgttggactctgacggtagtttttttctctattccaagctgactgtggacaaaagtcgatggcagcaggggaatgtgttctcttgttccgtgatgcacgaggccctccacaaccactatacacagaaaagcctgagcctcagccccggtaag

SEQ ID NO: 67 - DNA, 3699 bp, Homo sapiens
  source 1..3699: mol_type = genomic DNA
gtgaagcagaactctccaagtctcagcgtacaggaaggtcggatctctattttgaattgcgactacaccaattcaatgttcgactactttctttggtacaaaaagtaccctgcggagggaccaacattcctgatttcaataagcagtatcaaagataaaaacgaggacggccggtttacagttttcttgaacaaaagtgctaaacacctgagcttgcacatcgtgccatctcagcccggcgacagtgctgtgtatttctgtgccgcaagtgataatgacatgcgctttggtgccggaacaaggctgactgtgaagcccattcagaacccagagcctgctgtgtatcagctgaaggatccccggagccaggacagcacactttgtctttttacagactttgattcccaaataaacgtcccaaagacgatggagtctggaacatttattaccgataaaacggtccttgatatgaaggcgatggacagcaagtctaacggggcaattgcttggtctaaccaaacttcattcacttgccaagatattttcaaagaaaccaatgctacctacccttcctccgatgtgccctgtgatgcaactctgacaggagggggcggcagtggcggcgggggatccggcggaggcgggtccgtcacccagactcccaagttccaggtactgaagactggtcagagcatgactctgcagtgtgcacaggacatgaaccacaatagtatgtattggtacaggcaggaccccggcatgggcctgagacttatctactactctgcctcagagggaactacagataaaggcgaggtaccaaacggctataacgtgtctcgcctcaataaacgggaattcagcttgcggctcgaaagcgccgctcctagccagacaagcgtctacttctgcgcaaccaagagcagcctcacttatgaacagtacttcgggcctgggacaagactgacagtcactgaagaccttcggaatgttaccccccccaaagtcagtctgtttgaaccgtctaaagccgagatcgctaacaaacaaaaagccacactggtgtgcttggcccgcgggtttttccctgaccatgtcgaactcagttggtgggtgaatggtaaggaagtccactctggcgtgtccactgatccacaggcgtataaagaaagtaattatagctattgtctctcctctcgccttagagtatctgctacattctggcataaccccaggaaccactttaggtgtcaggtgcagtttcatggcctgtctgaagaggataaatggcccgagggctctcctaagccagtgacccagaatatctccgccgaagcctgggggcgggctgattgcggaatcactagcgcgggtggtgggggctctgacaaaacccacacctgtccgccttgcccagcccccgaagcagctggcggaccctctgttttcctgtttccaccaaagccaaaggacaccctgatgataagcagaacaccagaagtgacatgtgtagtggtggacgtgagccacgaagatcctgaagtgaagtttaattggtacgtggacggcgttgaggtgcacaacgccaaaactaaaccctgtgaggagcaatatggctctacttaccggtgtgtgtctgtcctgacagtgctgcaccaggactggctgaacgggaaagaatataagtgcaaggtttcaaataaggccctgcctgctcctattgaaaagacaatcagcaaagcgaagggacagcctcgggaacctcaggtttatacgctcccccccagccgcgaggaaatgactaagaaccaggtttctctgacatgcctcgttaaaggattttatccttcagacatcgccgtagagtgggagtccaacggccagccagaaaataactacaagaccactccccccgtacttgacagcgacgggagtttcttccttacatcaaagctgacggtcgataagagccggtggcagcaggggaatgtgttttcctgcagcgttatgcacgaggctctgcataatcactacactcagaaatccctgtcactgtccccagaatctaaatatggcccaccttgtcccccatgtcctgcacctgaggctgctggggggccaggtgggggagggagtgggggcggcgggagtgggggaggtgggagcggcggcgggggcagtggtgggggaggatctggcggaggaggctctgataagactcatacttgccccccgtgtccagcccccgaggccgccggcggaccgtccgttttcctctttccacccaagccgaaggataccttgatgatctcccgcactcccgaagtgacctgcgtagtcgtggatgtcagccacgaggatcctgaggtgaagtttaattggtatgtcgatggcgttgaagtgcataacgccaagacaaagccctgcgaggaacagtatgggtccacctacagatgtgttagtgtcctcacagtcttgcaccaagactggctgaacgggaaggagtataagtgtaaggtaagcaataaggcccttccagcaccaattgagaagactatcagcaaagccaaaggacagcctcgggaaccacaagtctacacgttgcctcccagtcgagaagaaatgaccaagaatcaagtttccctgtattgcctcgtcaaaggtttctacccctccgatattgctgtagaatgggagtcaaatggccaacctgagaataattacaaaactacgccccctgtcctggactcagatggctcctttttcctgtattctaagctgaccgtggacaagtctagatggcagcagggaaatgttttttcttgctcagtcatgcatgaggctctccacaaccattacacacaaaaaagtctctctttgtctccgggaaaggaatctaagtatggtccaccctgtcctccctgtccagctcctgaagcagcgggggggcccggaggcggaggatccgaggtacagctggtggagtcaggaggtggcctggtccagccaggtggctccctgcgcctgagctgcgctgcatcagggtactcctttacaggctatacaatgaactgggtgaggcaggcccccggcaagggactcgagtgggttgccctgataaacccatataagggcgtgtctacatacaaccagaagttcaaggatagatttacaatatccgtcgacaaaagtaagaacacagcatacctgcaaatgaatagtctgagagccgaagatactgccgtgtattactgcgcccggagcggttactacggggacagcgactggtattttgatgtgtggggtcaggggacccttgttacagtaagctctggaggcggggggtccggcgggggcgggtctggaggagggggctcagcaattcagatgacccaaagccccagttccctgtccgctagtgtgggcgatcgcgtgaccatcacatgtcgcgcctctcaggacatccgcaattatctgaattggtaccagcagaaaccaggcaaagccccaaaactgctcatttattatacttccagattggagagcggggtcccgtctaggttctctggaagcggctccgggaccgattatactcttactatttcctcactgcagcccgaagattttgctacatattattgccagcaggggaatactctgccatggacattcggacaggggactaaggttgagatcaag

SEQ ID NO: 68 - DNA, 2997 bp, Homo sapiens
  source 1..2997: mol_type = genomic DNA
gaagcagccgtgacccagagcccacgcaacaaggtcgctgtgaccggtgggaaggtcacactgcagtgcgcccaggatatgaacaaccacaataacatgtattggtatagacaggatactggatgtggtctgagactgatccactattctgcgagcgaaggcaccacagaaaagggcgatatcccggacggctacaaggcttcacggcctagtcaggagaatttttcactcatactggaactcgctacaccgtctcaaaccagtgtctacttctgcgccaccaagtcatccttgacgtatgaacagtacttcgggcctggtaccaggctcaccgtcctggagggtgggggaggatcaggaggcggaggctcctgcccaccctgtggcggcggagggagccaacagaaggtgcagcagtctccggagtctcttatcgttcccgaaggcggaatggcctccctgaactgtaccagtagtaacagcatgtttgatttttggtggtacagacagcacagcggcaagtcccctaaaatgttgatgtctatttcttccattaaagacaaaaacgaagagggcaggtttactgtacatctgaacaaggcaagtctgcatactagcctgcatataagagactcccagccttctgactcagccctgtacttgtgtgcggcttcagacaacgacatgagattcggatgcgggaccaagctcatcattaagccagacggggggggtggctccgaggttcagctggtggaaagcggcggaggattggtccaacccggaggcagtctgagactgtcttgcgcagctagtggttacagctttaccggatataccatgaattgggtacggcaagctccgggctgtgggttggagtgggtggcactcataaacccttacaagggcgtgtctacctacaaccagaaatttaaggatcgcttcactatctccgtggacaagtctaagaacacggcttaccttcagatgaactccctcagagctgaggataccgctgtttactattgcgcccgctcaggatattatggcgactctgactggtatttcgatgtgtggggacaaggcactctcgtgactgtgagtagcggcggtggtggttccggtgggggcggttcctgccccccatgtggaggggggggctccgccatccagatgactcaaagcccctcatcactgtctgcaagtgtgggagatagggtgaccataacttgcagggcctcccaggatattcgcaactacctgaattggtaccaacagaagccaggaaaggctccgaaactgctgatctactacaccagcagacttgagtccggggtaccgtccagattcagcggctctggcagtggcacagattatacattgacgatctcctctctgcagcccgaggattttgcgacatactactgccaacagggcaatacactcccgtggacttttggatgtggaaccaaggtggagattaagggagggggtggctcagataaaacacacacctgccctccctgtcctgcccctgaggcagccggtggtccaagtgtcttcctgttcccccctaaaccgaaagacactctgatgatatctcggactcctgaggtaacttgtgtcgtggtagacgtaagccacgaagaccccgaggtgaagttcaactggtatgttgacggagtggaagtgcacaacgccaagacaaagccctgtgaagaacagtatggcagcacctatcgatgcgtctctgtgttgacagttctgcaccaggactggctcaatggtaaagaatacaaatgtaaggtcagcaacaaagctctgcctgcccccattgaaaaaaccatcagcaaggccaaggggcagcctcgagaacctcaggtgtacactctgcctccgagccgagaggagatgactaagaaccaggtgtcactgacttgcctggtgaaaggtttctatccatctgacattgctgtggaatgggaatctaacgggcaaccagaaaacaactataagaccacccctcccgttttggacagtgacggtagctttttcctgaccagcaagctcaccgtagataagagtcgctggcagcagggtaacgtgttcagttgctctgtgatgcacgaggcactgcacaaccactacacacagaaatcattgtctctctctcccgggggaggtggatctggaggcggcggctccggtggtggcgggtctggtggggggggttcaggcggaggggggtctggaggcggggggagcgataaaacacatacctgccccccctgtccggcaccagaggcagctggaggcccatccgtttttctctttccccccaaacccaaagacacactcatgatatctagaacaccagaggtcacatgtgttgtagtggacgtgagtcacgaggaccccgaggtaaagtttaactggtatgtggacggcgtggaagtccacaacgccaagacaaaaccttgcgaagagcagtacggaagtacgtatagatgtgttagcgtgctgactgtcctccatcaagactggctcaacggaaaggagtataaatgtaaagttagcaataaagcattgcccgcgccaattgaaaagaccatttccaaagctaaggggcaaccacgggagcctcaagtgtatacacttccccccagcagagaggaaatgaccaagaaccaggtgtctctgtactgcctggtcaaaggcttttatcctagcgacatcgccgtggagtgggagtcaaacggccagccagaaaacaattataagaccaccccaccagtgctggacagtgacggatcattcttcctgtactccaaactgacagtcgacaagagtaggtggcagcagggaaatgtgttttcctgttccgttatgcacgaagcgctgcacaaccactatacacagaaatccctgtcactgtcccccggtaaggaaggtaggggatctcttctcacctgcggtgatgtcgaggagaatcctggc

SEQ ID NO: 69 - DNA, 2934 bp, Homo sapiens
  source 1..2934: mol_type = genomic DNA
gaagcagcggttacccagtcacccaggaataaggtggcagtgaccgggggcaaggttacacttcagtgcgcacaggacatgaataatcacaataatatgtactggtaccgccaggataccggttgtgggctgaggttgatccactattcagccagtgagggtaccacagagaagggggatatacctgacggttataaagcctccagaccttctcaggaaaacttttccctcatactggaacttgcaacaccaagtcagacgagtgtctacttttgcgcaaccaaaagctctcttacctacgaacaatactttggcccaggtaccagactcacagttctcgagggaggcggaggcagcgggggcggcggatcctgtcctccgtgtggtggcggtggctcacagcaaaaggtccaacagagcccagaatcactgattgtgcctgaagggggaatggcttcactcaattgtaccagtagcaattcaatgttcgacttctggtggtacaggcagcattcaggaaaatctcctaagatgctgatgtccatctcttccattaaggacaagaacgaagaagggcgcttcacagtgcatctcaacaaggcctctctgcatacgtcactccacatccgcgactcacaacctagcgattcagccctttacctgtgtgccgcaagtgacaacgacatgcgatttggctgtgggactaaactcattattaaacccgatggcggcggcgggagtgacaaaacgcacacatgccccccttgcccagcccccgaggccgcggggggtccttctgtgtttctgttcccccctaagcccaaagacacacttatgatatctagaacaccagaggtgacttgtgtggtggttgacgtgtcccacgaggaccccgaagtcaaattcaactggtatgtagacggcgtggaggtacacaacgccaaaaccaagccatgcgaggagcagtacggctccacgtaccgatgtgtcagtgtgctgaccgtgctgcaccaggactggcttaacggcaaggaatataagtgtaaggtttctaataaagccctgccagcgcccattgaaaagaccatttccaaggccaagggtcagcccagggagccccaggtttacacacttcctccgagcagggaggaaatgactaagaatcaggtcagcctcacatgtctcgtcaaagggttttatccctccgacattgcggtcgagtgggaatccaacgggcagccagagaataattacaaaactaccccaccagtactggattctgacggctcattttttcttacctcaaaactgaccgtggataaatccagatggcagcaaggtaacgtcttttcttgtagtgtgatgcatgaggccctgcataaccactacacacagaaatccctctcattgagtcccgggggaggaggcagtggcgggggtgggagcggcggtggtggtagcggtggaggcggcagcggagggggcggatctgggggaggtggctctgataaaacccatacttgccctccttgcccagcccccgaggccgccggagggccttcagtcttcttgttcccacctaagcccaaagatacccttatgataagcagaacaccagaggtgacctgcgtggtcgtggatgtgtcccatgaggacccagaagtgaagttcaactggtacgtggatggtgtcgaagtgcacaacgccaagaccaaaccttgtgaggaacagtacggcagtacttaccgctgcgtctccgtgctcacagtgcttcaccaggattggctgaatggaaaggagtacaagtgcaaagtgtctaacaaggctctcccagcccctattgagaaaactatatccaaagccaaaggtcagcctcgcgagccccaggtctacacgctcccacctagtcgcgaggagatgaccaaaaatcaagtgtccctttattgcctggtcaagggcttttacccttccgatatcgcagtcgaatgggagtctaatgggcaacccgagaacaactataaaacaacaccgccggtgctggatagtgacggtagttttttcttgtatagcaagctgaccgtcgataagtcccggtggcaacaaggcaacgtcttctcctgttctgtgatgcacgaagccctgcacaaccactatacccagaagagtctgagcctgtcccccgggaagggagggggaggttctgaggttcaactcgtcgagtcaggaggcggcctcgtccagcccggaggtagtctgcggttgtcatgtgcagcctccggttacagttttactggctacacgatgaattgggtgagacaggcacctgggaagggcttggaatgggttgccctgattaacccatacaaaggcgtatcaacctacaaccaaaaattcaaagaccgcttcaccatctcagttgataaaagcaaaaacacagcttatttgcagatgaacagccttagggctgaagatacagctgtatattattgtgcgagaagcggctattatggggactccgattggtacttcgatgtctggggccagggtactctggtgacagtgagcagcgggggaggaggctcaggcggtggcggctccggaggcgggggttcagccatccagatgacgcagtcacccagctccctcagcgcctctgtcggagacagagtaaccatcacttgcagggcctcccaggatatccgcaattacctgaactggtaccaacagaagcctgggaaggccccaaaactgctgatctattatacatctcgccttgagtctggcgttccgagcaggtttagcggttctggaagcggaactgactacaccctcacaatcagctctctgcagcctgaagattttgccacatactactgccagcagggaaacaccctgccttggacctttggccaagggacgaaggtcgaaatcaag

SEQ ID NO: 70 - protein, 25 aa, Homo sapiens
SGNLLGRNSFEVHVCACPGRDRRTE

SEQ ID NO: 71 - protein, 10 aa, Homo sapiens
LGRNSFEVHV

SEQ ID NO: 72 - protein, 10 aa, Homo sapiens
NSFEVHVCAC

SEQ ID NO: 73 - protein, 10 aa, Homo sapiens
RNSFEVHVCA

SEQ ID NO: 74 - protein, 9 aa, Homo sapiens
SFEVRVCAC

SEQ ID NO: 75 - protein, 9 aa, Homo sapiens
SFEVCVCAC

SEQ ID NO: 76 - protein, 9 aa, Homo sapiens
SFEVLVCAC

SEQ ID NO: 77 - protein, 9 aa, Homo sapiens
SFEVGVCAC

SEQ ID NO: 78 - protein, 9 aa, Homo sapiens
SFEVPVCAC

SEQ ID NO: 79 - protein, 9 aa, Homo sapiens
SFEVSVCAC

SEQ ID NO: 80 - protein, 9 aa, Homo sapiens
SFEIHVCSC

SEQ ID NO: 81 - protein, 9 aa, Homo sapiens
SFEGRICAC

SEQ ID NO: 82 - protein, 9 aa, Homo sapiens
SFQCGVCAC

SEQ ID NO: 83 - protein, 9 aa, Homo sapiens
SFGVHRCAD

SEQ ID NO: 84 - protein, 9 aa, Homo sapiens
SFEARICAC

SEQ ID NO: 85 - protein, 9 aa, Homo sapiens
SFSIQVCAC

SEQ ID NO: 86 - protein, 9 aa, Homo sapiens
CVCVHVCAC

SEQ ID NO: 87 - protein, 9 aa, Homo sapiens
CFEARICAC

SEQ ID NO: 88 - protein, 9 aa, Homo sapiens
KFESNVCVC

SEQ ID NO: 89 - protein, 9 aa, Homo sapiens
LFNIHVSAC

SEQ ID NO: 90 - protein, 9 aa, Homo sapiens
FSVPHVCAR

SEQ ID NO: 91 - protein, 9 aa, Homo sapiens
SFTTHSCAP

SEQ ID NO: 92 - DNA, 2265 bp, Homo sapiens
  source 1..2265: mol_type = genomic DNA
ggtagaggaggcggcgggagtgaagttcaactggtggagagcggcggtggcctggtgcaacctgggggctcactgcgcctgtcttgtgctgctagcggatacagttttaccgggtacaccatgaactgggtgcgacaagcacccggaaagggcctcgaatgggtggcacttataaacccatacaaaggcgtgagcacatacaaccagaaattcaaagataggttcactatcagtgtggataaaagcaaaaacacagcttatctccaaatgaactcactgcgggccgaagataccgcagtgtactattgcgctcgcagcgggtactacggcgatagtgattggtatttcgacgtatggggacaagggacactggtgacagtcagcagtggaggcggcggcagcggaggtggtggctcaggagggggaggttctgccattcagatgactcaatctccatcctccctctcagcctcagtcggcgatcgggtcacgatcacttgtcgggccagtcaggatatcaggaattaccttaactggtaccagcagaagccgggtaaggcaccaaagttgcttatctattatacttccagactggagtctggggtccctagtcggttttctggctctggcagcgggaccgattacacgctcacgattagcagcctgcagcccgaggacttcgctacatactattgtcagcagggtaatactctgccctggactttcggccaggggaccaaagtggagatcaagggcggaggagggtccgataaaacccatacctgcccgccgtgccccgcccctgaagcagccggcgggccctccgtttttctgtttccccctaagcccaaagataccctcatgatcagcaggacacccgaggtgacatgcgtagtggtggacgtgtcccacgaggaccccgaggtgaagttcaactggtacgttgacggagtggaggtgcacaacgccaagaccaagccctgcgaagagcaatatggtagcacgtaccggtgcgtttcagtactcactgtcctccaccaagactggctgaacgggaaggagtacaaatgtaaagtctccaacaaagcactgcctgctcccatcgaaaagaccatttctaaggctaagggccaaccacgcgaacctcaagtttatacactccccccatcaagggaggaaatgaccaagaaccaagtgtccctcacctgtctcgtaaagggcttctatccttcagatatagcagttgagtgggaatccaacggccagcctgagaacaattataagacaacccctcctgttctggactctgatggaagtttcttcttgacctctaagctgacagtggataagtcccgctggcagcagggaaacgtgtttagttgctctgtgatgcatgaagctctgcacaaccattatacccagaagtccctgtctttgtcaccaggaggaggtggcagcggtgggggcggctccgggggtggcggtagcggcgggggggggtccggcggtgggggatctgggggcggtggctctgataagactcacacttgtcccccttgtccagcacccgaagcagccggtgggccctccgtgttcctgttcccacctaaaccaaaggacactcttatgatttccagaacccccgaggtaacctgtgttgtcgtagatgttagccatgaagatcctgaagtgaaattcaattggtacgtggacggtgtcgaggtacacaacgccaaaactaaaccctgcgaggagcagtacgggtcaacttatcggtgtgtgagtgtgcttactgtgctccatcaggattggctgaacgggaaagagtataaatgcaaagtgagtaacaaagcactccccgcgccaatcgagaagaccatcagcaaagctaagggacaaccccgagaaccccaagtatacacactgccccctagccgcgaagagatgaccaaaaaccaagtcagcttgtattgcctggttaaaggcttttacccctcagatattgcagtggaatgggagtcaaatggacagccagagaacaactacaaaaccacaccgccagtccttgactccgatgggtccttttttctgtatagcaagctgacagtggataaaagcagatggcagcagggcaatgtgttttcctgtagcgttatgcacgaagccttgcataatcattacactcagaagagcttgtcactttctccaggtaaggagggacgagggagcctgctgacatgcggcgacgtggaagaaaacccagga

SEQ ID NO: 93 - protein, 755 aa, Homo sapiens
GRGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPG

SEQ ID NO: 94 - DNA, 2265 bp, Homo sapiens
  source 1..2265: mol_type = genomic DNA
ggccggggtggaggagggagcgacaaaacgcatacttgtccaccttgtcccgcaccagaggcagcgggaggtcccagcgtgttcctgttcccacctaagcccaaggatacacttatgatcagcagaacgcccgaggtcacctgcgttgttgtggatgtgagtcacgaggatcccgaggtgaagtttaattggtacgtggatggagtcgaagtgcacaatgccaagacaaaaccctgtgaagaacaatatggaagcacatacagatgcgtttccgtcttgactgtcctgcaccaggattggttgaacggaaaagaatataaatgcaaggtttccaataaggctctccctgcgccgatcgagaagactatatcaaaggctaaaggccagccccgcgaaccacaggtctataccctccctccatcaagagaggaaatgaccaagaaccaagttagcttgacttgcctggtcaagggattctatccctctgatattgcagttgagtgggagtctaacggacagcctgagaataactataagaccacccctccagttcttgacagtgacggatcattttttctgacatctaagctgaccgtggacaagtcccgctggcagcagggcaacgtcttctcctgctctgtaatgcatgaagcacttcacaaccactatacacagaagagcttgagcctttctccaggtggcggcggaagcggcggtggggggagtggtggcggaggatccggaggggggggatctggaggtggtggctctggcggtgggggctccgacaaaacccatacatgcccaccctgtcctgcgcccgaggccgccggaggtccgagcgtctttctgttcccccccaagcccaaagacactctgatgatctcccggacccccgaagtgacctgtgtcgtggttgatgtgtcccatgaagaccccgaggtcaaattcaattggtacgtggacggcgtagaggttcacaatgctaagactaagccctgcgaagagcaatacggctctacatatcgctgcgtaagcgtcctgacagttcttcaccaggactggttgaatgggaaggagtataaatgtaaggtatcaaataaggccctcccagcgccaatagaaaagactattagcaaggccaagggccaacccagggagccccaggtgtacaccctgccgccgtctagagaggagatgaccaagaaccaggtaagtttgtattgcctggttaagggtttttatccaagtgatatcgctgtggaatgggaatcaaatggtcagcctgagaacaattataaaacgaccccacctgtccttgattcagacggctccttctttctgtattccaagcttacggttgataagtcaaggtggcaacaaggaaacgtttttagttgcagcgtcatgcatgaggccctgcacaatcattacacacagaaatctctgagcttgtctccaggcaagggtggaggaggctcagaagtgcagctcgtcgagagtgggggaggcttggtccagcccggcgggagtttgcgactgtcctgcgccgccagcggctactcattcacaggctacacaatgaactgggtcagacaggccccagggaaagggctggagtgggtcgcattgattaatccatataaaggcgtgtccacgtataatcagaaatttaaggaccgctttacgatcagtgtggataagagtaaaaacaccgcgtatctgcagatgaattcactccgcgccgaggacaccgcagtctactattgcgcaagatcaggttattatggggattcagactggtactttgacgtttggggccagggaactctggtaactgtctcctccgggggtggcggatctgggggaggtggatctggtggtggaggaagtgcgatccagatgacacagagcccctcctccttgagcgcctctgtgggtgacagggtgactattacttgccgcgctagccaggacattaggaactacctgaactggtatcagcagaaaccgggaaaagcacccaaattgctgatctactatacatctagattggagtccggcgtcccgtccagattttcaggctcaggaagtggcaccgactacaccctgacaatcagcagtttgcaacccgaggactttgcaacttattactgccagcagggtaacacacttccctggacattcgggcaaggtacgaaggtggagattaaggaaggcaggggtagcctgctgacttgtggagatgttgaggagaacccgggg

SEQ ID NO: 95 - protein, 755 aa, Homo sapiens
GRGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKEGRGSLLTCGDVEENPG

SEQ ID NO: 96 - protein, 364 aa, Homo sapiens
AVMAPRTLVLLLSGALALTQTWAGSHSMRYFYTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 97 - protein, 386 aa, Homo sapiens
AVMAPRTLVLLLSGALALTQTWAGSHSMRYFYTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWDYKDDDDKHHHHHHDYKDDDDKEPSSQPTIPIVGIIAGLVLFGAVITGAVVAAVMWRRKSSDRKGGSYSQAASSDSAQGSDVSLTACKV

SEQ ID NO: 98 - protein, 478 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSAVMAPRTLVLLLSGALALTQTWAGSHSMRYFYTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 99 - protein, 500 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSAVMAPRTLVLLLSGALALTQTWAGSHSMRYFYTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWDYKDDDDKHHHHHHDYKDDDDKEPSSQPTIPIVGIIAGLVLFGAVITGAVVAAVMWRRKSSDRKGGSYSQAASSDSAQGSDVSLTACKV

SEQ ID NO: 100 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 101 - protein, 383 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWDDDDKHHHHHHDYKDDDDELSSQPTIPIVGIIAGLVLLGAVITGAVVAAVMWRRKSSDRKGGSYTQAASSDSAQGSDVSLTACKV

SEQ ID NO: 102 - protein, 479 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSMAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 103 - protein, 497 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSMAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWDDDDKHHHHHHDYKDDDDELSSQPTIPIVGIIAGLVLLGAVITGAVVAAVMWRRKSSDRKGGSYTQAASSDSAQGSDVSLTACKV

SEQ ID NO: 104 - protein, 361 aa, Homo sapiens
RVTAPRTVLLLLSAALALTETWAGSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 105 - protein, 379 aa, Homo sapiens
RVTAPRTVLLLLSAALALTETWAGSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWDDDDKHHHHHHDYKDDDDEPSSQSTVPIVGIVAGLAVLAVVVIGAVVAAVMCRRKSSGGKGGSYSQAACSDSAQGSDVSLTA

SEQ ID NO: 106 - protein, 475 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSRVTAPRTVLLLLSAALALTETWAGSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 107 - protein, 493 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSRVTAPRTVLLLLSAALALTETWAGSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWDDDDKHHHHHHDYKDDDDEPSSQSTVPIVGIVAGLAVLAVVVIGAVVAAVMCRRKSSGGKGGSYSQAACSDSAQGSDVSLTA

SEQ ID NO: 108 - protein, 361 aa, Homo sapiens
RVTAPRTLLLLLWGALALTETWAGSHSMRYFYTAMSRPGRGEPRFIAVGYVDDTQFVRFDSDAASPRGEPRAPWVEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAARVAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 109 - protein, 380 aa, Homo sapiens
RVTAPRTLLLLLWGALALTETWAGSHSMRYFYTAMSRPGRGEPRFIAVGYVDDTQFVRFDSDAASPRGEPRAPWVEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAARVAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWDDDDKHHHHHHDYKDDDDKEPSSQSTIPIVGIVAGLAVLAVVVIGAVVATVMCRRKSSGGKGGSYSQAASSDSAQGSDVSLTA

SEQ ID NO: 110 - protein, 475 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSRVTAPRTLLLLLWGALALTETWAGSHSMRYFYTAMSRPGRGEPRFIAVGYVDDTQFVRFDSDAASPRGEPRAPWVEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAARVAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 111 - protein, 494 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSRVTAPRTLLLLLWGALALTETWAGSHSMRYFYTAMSRPGRGEPRFIAVGYVDDTQFVRFDSDAASPRGEPRAPWVEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAARVAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWDDDDKHHHHHHDYKDDDDKEPSSQSTIPIVGIVAGLAVLAVVVIGAVVATVMCRRKSSGGKGGSYSQAASSDSAQGSDVSLTA

SEQ ID NO: 112 - protein, 369 aa, Homo sapiens
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWDYKEPSSQPTIPIVGIVAGLAVLAVLAVLGAVVAVVMCRRKSSGGKGGSCSQAASSNSAQGSDESLIACKA

SEQ ID NO: 113 - protein, 388 aa, Homo sapiens
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWDYKDDDDKHHHHHHDYKDDDDKEPSSQPTIPIVGIVAGLAVLAVLAVLGAVVAVVMCRRKSSGGKGGSCSQAASSNSAQGSDESLIACKA

SEQ ID NO: 114 - protein, 483 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSMRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWDYKEPSSQPTIPIVGIVAGLAVLAVLAVLGAVVAVVMCRRKSSGGKGGSCSQAASSNSAQGSDESLIACKA

SEQ ID NO: 115 - protein, 502 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSMRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWDYKDDDDKHHHHHHDYKDDDDKEPSSQPTIPIVGIVAGLAVLAVLAVLGAVVAVVMCRRKSSGGKGGSCSQAASSNSAQGSDESLIACKA

SEQ ID NO: 116 - protein, 368 aa, Homo sapiens
RVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWDYKEPSSQPTIPIMGIVAGLAVLVVLAVLGAVVTAMMCRRKSSGGKGGSCSQAACSNSAQGSDESLITCKA

SEQ ID NO: 117 - protein, 387 aa, Homo sapiens
RVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWDYKDDDDKHHHHHHDYKDDDDKEPSSQPTIPIMGIVAGLAVLVVLAVLGAVVTAMMCRRKSSGGKGGSCSQAACSNSAQGSDESLITCKA

SEQ ID NO: 118 - protein, 482 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWDYKEPSSQPTIPIMGIVAGLAVLVVLAVLGAVVTAMMCRRKSSGGKGGSCSQAACSNSAQGSDESLITCKA

SEQ ID NO: 119 - protein, 501 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGSGSGGGGGSRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWDYKDDDDKHHHHHHDYKDDDDKEPSSQPTIPIMGIVAGLAVLVVLAVLGAVVTAMMCRRKSSGGKGGSCSQAACSNSAQGSDESLITCKA

SEQ ID NO: 120 - protein, 244 aa, Homo sapiens
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 121 - DNA, 732 bp, Homo sapiens
  source 1..732: mol_type = genomic DNA
gaggtgcaattggtagaaagtggaggcggtcttgtccagcctggtggctcattgcggctttcttgtgctgccagcggctactccttcaccgggtataccatgaactgggtgaggcaagcacccggtaagggcctggaatgggtcgccttgatcaacccctataaaggagtctccacttataaccagaagtttaaggatcggtttaccattagcgtcgataaatctaaaaataccgcctacttgcaaatgaactcactgcgcgccgaagatactgcagtctactactgtgctaggagtggctactatggagattctgattggtacttcgacgtctggggccagggtacattggtgaccgtgagcagcggcggagggggcagtggtggaggtggctctggcggcggcggttccgccatacaaatgacacagtccccctcttcactgagcgcatccgtaggggacagagtcaccatcacatgccgcgcatcccaggacataagaaactacctgaactggtatcagcagaagccaggcaaggctcctaaacttttgatttattataccagcaggctggagtcaggtgttccaagcagattttcaggaagcggctccggcaccgactatacccttacgatctctagtctccagcctgaggactttgcaacctactattgtcagcagggaaataccctcccatggacattcggacagggcacaaaggtggagattaag

SEQ ID NO: 122 - protein, 248 aa, Homo sapiens
EVQLVESGGGLVQPGGSLRLSCAASGYSFTGYTMNWVRQAPGCGLEWVALINPYKGVSTYNQKFKDRFTISVDKSKNTAYLQMNSLRAEDTAVYYCARSGYYGDSDWYFDVWGQGTLVTVSSGGGGSGGGGSCPPCGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 123 - DNA, 744 bp, Homo sapiens
  source 1..744: mol_type = genomic DNA
gaagttcagctcgttgagtctggcggtggactcgtccagccaggaggtagtctgcggctgtcctgtgcagcctcaggctattcttttacaggatataccatgaactgggttaggcaggcaccaggatgcggtctggaatgggtagctctgattaatccgtacaagggtgtaagcacttataaccaaaagttcaaggataggtttacaattagcgtcgataaatctaagaatacggcatatctgcaaatgaatagtttgcgagccgaggacactgcagtctattactgtgcccgatctggatattacggtgattccgactggtatttcgacgtgtgggggcagggaaccctcgtcacagtttcctcaggcgggggaggtagtggcgggggtgggagctgcccaccttgcggtggtggaggatccgccatacaaatgacacaatctccatcatctctgtccgcaagcgtgggcgacagggtgaccataacctgtcgagcctcacaggacattagaaattacctgaattggtatcaacagaagcctggtaaggccccaaaattgctcatctattataccagcaggctggagtccggtgtcccttcacgattcagtggatccggcagcggcaccgactacacgcttaccatatccagcctgcagcccgaagatttcgcaacttactattgccaacagggaaatacactcccttggaccttcggctgtggaacgaaggtggagatcaag

SEQ ID NO: 124 - protein, 482 aa, Homo sapiens
DKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 1446 bp, Homo sapiens
  source 1..1446: mol_type = genomic DNA
gacaaaacacatacatgccccccttgcccagcccccgaggcagctggcgggccatctgtatttctctttccacccaagccaaaagatacactcatgataagcagaaccccggaagtcacctgtgtagtggtcgacgtctctcacgaagatcccgaagtcaagttcaactggtatgtggacggagtcgaggtgcataacgccaaaacaaaaccatgtgaagaacaatacggatccacatatcgctgcgtgagcgtgcttaccgtcttgcaccaggactggctgaacggtaaagaatacaaatgtaaggtttccaataaagctctgccagcccccattgagaaaacaatctcaaaggctaaggggcagccccgggaacctcaggtctatacattgccacccagtcgagaggagatgaccaagaaccaagtgtccctgacctgcctcgtgaagggattctatcccagcgatatcgctgtggaatgggagtccaatggacagcctgagaataactataagaccacaccgcctgtcctcgatagcgacggctctttcttcctgacctcaaaactgactgtcgataagagccggtggcagcaggggaatgttttctcatgttctgtgatgcacgaagccctccacaatcattatacgcagaagagcctttccctctccccgggagggggaggatctggtggtggcggatccggcgggggaggaagtgggggaggtgggagcggagggggcggaagtggcggcggaggttccgacaagactcacacatgcccaccctgcccggcccctgaggcggccggaggcccttctgtgttccttttcccaccaaaacccaaagatacgcttatgattagtcggacaccagaggtgacatgtgtcgtggtagatgtatcccacgaggacccggaggtaaaattcaattggtatgtggatggtgtagaagtccacaacgcgaaaacaaaaccttgtgaggaacagtatggatccacctatcggtgtgtgagcgtactgacggtacttcatcaggattggctcaatggtaaggaatataagtgcaaagttagtaataaggccctgcctgcccccattgaaaagaccatatctaaagctaaaggacaaccacgggagccacaggtatacaccttgccaccatcacgagaagagatgaccaaaaaccaagtgtctctttactgtcttgtgaaaggattctacccgtcagacatagccgttgagtgggagagcaatggccaacccgagaacaattacaagactactccacctgtgttggacagtgatggatcctttttcttgtatagtaaacttactgttgataagagccgatggcaacagggcaatgtgtttagttgctctgtgatgcacgaagctctccacaatcattatacccagaaaagcctctctttgagccccggaaag